(19) United States
(12) Patent Application Publication
(10) Pub. No.: US 2013/0236553 A1
(43) Pub. Date: Sep. 12, 2013

(54) Compositions and Methods for the Delivery of Therapeutics

(71) Applicants: Howard E. Gendelman, Omaha, NE (US); Alexander V. Kabanov, Chapel Hill, NC (US); Xin-Ming Liu, Omaha, NE (US)

(72) Inventors: Howard E. Gendelman, Omaha, NE (US); Alexander V. Kabanov, Chapel Hill, NC (US); Xin-Ming Liu, Omaha, NE (US)

(73) Assignee: Board of Regents of the University of Nebraska, Lincoln, NE (US)

(21) Appl. No.: 13/880,819

(22) PCT Filed: Nov. 2, 2011
(86) PCT No.: PCT/US11/58929

§ 371 Date: May 20, 2013

Related U.S. Application Data
(60) Provisional application No. 61/409,372, filed on Nov. 2, 2010.
(60) Provisional application No. 61/526,976, filed on Aug. 24, 2011.

(51) Int. Cl. A61K 9/14; A61K 45/06; A61K 31/496; A61K 31/427; A61K 38/05; A61K 31/536

(52) U.S. Cl. CPC A61K 9/14; A61K 38/05; A61K 31/536; A61K 31/496; A61K 31/427; A61K 45/06
USPC 424/490; 514/3.7; 514/230.5; 514/253.01; 514/365; 514/3.8; 424/400

(57) ABSTRACT

The present invention provides compositions and methods for the delivery of therapeutics to a cell or subject.

This application claims priority under 35 U.S.C. §119(e) to U.S. Provisional Patent Application No. 61/409,372, filed Nov. 2, 2010 and U.S. Provisional Patent Application No. 61/526,976, filed Aug. 24, 2011. The foregoing application is incorporated by reference herein.

This invention was made with government support under Grant No. 1PO1DA026146-01 awarded by the National Institutes of Health/National Institute on Drug Abuse. The government has certain rights in the invention.

### FIELD OF THE INVENTION

The present invention relates generally to the delivery of therapeutics. More specifically, the present invention relates to compositions and methods for the delivery of therapeutic agents to a patient for the treatment of a viral infection.

### BACKGROUND OF THE INVENTION

The need to improve the bioavailability, pharmacology, cytotoxicities, and interval dosing of antiretroviral medications in the treatment of human immunodeficiency virus (HIV) infection is notable (Broder, S. (2010) Antivir. Res., 85:1-18; Este et al. (2010) Antivir. Res., 85:25-33; Moreno et al. (2010) J. Antimicrob. Chemother., 65:827-835). Since the introduction of antiretroviral therapy (ART), incidences of both mortality and co-morbidities associated with HIV-1 infection have decreased dramatically. It has been demonstrated that nanoformulated indinavir (IDV) can improve biodistribution and antiretroviral efficacy (Dou et al. (2006) Blood 108:2827-2835; Dou et al. (2009) J. Immunol. 183:661-669; Dou et al. (2007) Virology 358:148-158; Nowacek et al. (2009) Nanomedicine 4:903-917). However, many limitations associated with ART still remain which prevent full suppression of viral replication in HIV-infected individuals. These limitations include poor pharmacokinetics (PK) and biodistribution, life-long treatment, and multiple untoward toxic side effects (Garvie et al. (2009) J. Adolesc. Health 44:124-132; Hawkins, T. (2006) AIDS Patient Care STDs 20:6-18; Royal et al. (2009) AIDS Care 21:448-455). Since antiretroviral medications are quickly eliminated from the body and do not thoroughly penetrate all organs, dosing schedules tend to be complex and involve large amounts of drug. Patients have difficulty properly following therapy guidelines leading to suboptimal adherence and increased risk of developing viral resistance, which can result in treatment failure and accelerated progression of disease (Danel et al. (2009) J. Infect. Dis. 199:66-76). For HIV-infected patients who also experience psychiatric and mental disorders and/or drug abuse, proper adherence to therapy is even more difficult (Meade et al. (2009) AIDS Patient Care STDs 23:259-266; Baum et al. (2009) J. Acquir. Immune Defic. Syndr., 50:93-99). Accordingly, there is a need for drug delivery systems that optimize cell uptake, improve intracellular stability, extend drug release, maintain antiretroviral efficacy, and minimize cellular toxicity within transporting cells.

### SUMMARY OF THE INVENTION

In accordance with the instant invention, crystalline nanoparticles comprising at least one therapeutic agent and at least one surfactant are provided. In a particular embodiment, the surfactant is an amphiphilic block copolymer. In a particular embodiment, the surfactant is linked to at least one targeting ligand such as a macrophage targeting ligand. In a particular embodiment, the therapeutic agent is an antiviral, antiretroviral, or anti-HIV compound. Compositions comprising at least nanoparticle of the instant invention and at least one pharmaceutically acceptable carrier are also provided.

According to another aspect of the instant invention, methods for targeting therapeutic agents to an organ(s) and methods for treating, inhibiting, or preventing a disease or disorder in a subject are provided. In a particular embodiment, the method comprises administering to the subject at least one nanoparticle of the instant invention. In a particular embodiment, the method comprises targeting the therapeutic agent to the brain. In a particular embodiment, the methods are for treating, inhibiting, or preventing an HIV infection and the therapeutic agent of the nanoparticle is an anti-HIV compound. In a particular embodiment, the method further comprises administering at least one further therapeutic agent or therapy for the disease or disorder, e.g., at least one additional anti-HIV compound.

### BRIEF DESCRIPTIONS OF THE DRAWING

FIG. 1 provides images of nanoART morphology and cellular incorporation of nanoART. Scanning electron microscopy (SEM) analyses (magnification, 15,000×) of nanoformulations of IDVM1001-M1005), RTV (M2001-M2005), ATV (M3001-M3005), and EFV (M4001-M4005) on top of a 0.2 μm polycarbonate filtration membrane. All IDV nanoART were spherical to ellipsoid with rough edges; ritonavir (RTV) nanoART resembled thick rods with smooth edges; atazanavir (ATV) nanoART resembled thin rods with smooth edges; and efavirenz (EFV) nanoART were spherical to ellipsoid with rough edges. Transmission electron microscopy (TEM) (magnification, 15,000×) demonstrated uptake of nanoART into MDMs exposed to M1004, M2006, M3001, and M4002. Within the cells, each type of nanoART is readily identifiable by shape and an example has been outlined for IDV (M1004), RTV (M2006), ATV (M3001), and EFV (M4002). Measure bar in all frames equals 1.0 μm.

FIG. 2 provides timecourses of uptake of IDV, RTV, ATV, and EFV nanoART into monocyte-derived macrophage (MDM). Levels of IDV (FIG. 2A), RTV (FIG. 2B), ATV (FIG. 2C), or EFV (FIG. 2D) from cell lysates of cultured MDM treated with nanoART and collected at 1, 2, 4 and 8 hours were assayed by high performance liquid chromatography (HPLC). Data represent the mean±standard error of the mean (SEM) for n=3 determinations/time point.

FIG. 3 provides the area under the curve (AUC) of uptake of nanoART into MDM. AUC of uptake of IDV (FIG. 3A), RTV (FIG. 3B), ATV (FIG. 3C) and EFV (FIG. 3D) were determined in cell lysates of cultured MDMs treated with 100 μM nanoART and collected after 1, 2, 4, and 8 hours. Data represent the mean AUC for n=3 determinations/treatment.

FIG. 4 provides the scoring of nanoART formulations based on drug uptake, release, and anti-retroviral activity. ^{a}Uptake of drug based on the AUC of drug concentration in MDM over 8 hours. ^{b}Cell retention based on the AUC of drug concentration retained in MDM over 15 days. ^{c}Medium release is based on the AUC of drug concentration released into media over 15 days. ^{d}Antiretroviral activity determined from the AUC of reverse transcriptase (RT) activity from supernatant from infected MDM over 15 days. ^{e}GO/NOGO based on the mean of parameters that have been scored.

FIGS. 5A and 5B provide time courses of cell retention and release of IDV, RTV, ATV, and EFV nanoART. Levels of IDV, RTV, ATV or EFV in cell lysates and cell medium were assayed by HPLC on days 1, 5, 10 and 15 post-nanoART treatment. Data represent the mean±SEM for n=3 determinations/time point. For M1002-M1005, IDV levels were undetectable in the medium at day 15 (limit of detection: 0.025 μg/ml).

FIG. 6 shows the antiretroviral efficacy of nanoART. Comparison of antiretroviral effects in MDM challenged with HIV-1_{ADA} 15 days after pre-treatment with nanoART as measured by RT activity 10 days after viral challenge. RT activities were measured by ³H-TTP incorporation. Data represent mean for n=8 determinations/treatment.

FIG. 7 provides HIV-1 p24 antigen expression in nanoART treated cells. Comparison of antiretroviral effects of M1002 to M1004, M2002 to M2004, M3001 to M3005, and M4003 to M4005 challenged with HIV-1_{ADA} 1 to 15 days after pre-treatment with nanoART. Ten days after each viral challenge cells were immunostained for HIV-1 p24 antigen. Cells treated with both IDV formulations, M2002 (RTV), and M3005 (ATV) showed progressive loss of viral inhibition and increased HIV p24 expression over time; while cells treated with M2004 (RTV), M3001 (ATV), and both EFV formulations showed complete or greatly improved suppression of viral p24 production. However, even in nanoART treated cells where viral breakthrough did occur, p24 expression was less than HIV-1-infected cells that were not treated with nanoART.

FIG. 8 shows the characterization of the ritonavir nanoparticle and its cellular interactions. FIG. 8A shows RTV-NP with measurements of physical properties and depicting coating of an inner layer of mPEG₂₀₀₀-DSPE/188 and an outer layer of DOTAP. Size and charge were determined by dynamic light scattering. At least four iterations for each reading were taken with <2% variance. Scanning electron microscopy (magnification, 15,000×) of RTV-NP on top of a 0.2-μm polycarbonate membrane shows typical morphology resembling short rods with smooth edges (FIG. 8B). Uptake of RTV-NP in monocyte-derived macrophages (MDMs) over 12 hours and retention of RTV-NP within MDMs (left y-axis) and release of drug to surrounding media (right y-axis) over 15 days were determined by high-performance liquid chromatography (FIGS. 8C and 8D). Flow cytometry data and high-performance liquid chromatography data of MDMs exposed to fluorescent RTV-NPs demonstrate that treating MDMs with the clathrin inhibitor Dynasore significantly reduces uptake (FIGS. 8E and 8F). All data represent the mean±standard error of the mean for, n=3.

FIG. 9 shows the proteomic analyses of RTV-NP locale. Intracellular RTV-NP were identified within distinct membrane-bound compartments by transmission electron microscopy (magnification 15,000×) (FIG. 9A). FIG. 9B shows the subcellular localization process. RTV-NP were labeled with Brilliant Blue-250 and exposed to MDM. The cells were lysed and subcellular compartments separated by centrifugation on a sucrose gradient. Bands represent compartments that contain RTV-NP. These bands were collected, and the proteins separated by electrophoresis. Following in-gel trypsin digest, the proteins were identified using liquid chromatography/mass spectrometry. FIG. 9C shows the subcellular distribution of the identified proteins. A total of 38 endosomal proteins were identified. Proteomic analysis indicated that RTV-NP distribution was primarily with recycling endosomes (RE) and early endosome (EE) compartments.

FIG. 10 provides protein markers associated with ritonavir-nanoparticle-containing endosomes. ^{†}Number of unique significant (p<0.05) peptides identified for each protein. ^{‡}Theoretical molecular mass for the primary translation product calculated from DNA sequences. §Accession numbers for UniProt (accessible at www.uniprot.org). ¶Postulated subcellular localizations (see www.uniprot.org, locate.imb.uq.edu.au, and www.ncbi.nlm.nih.gov/pubmed). #Postulated cellular function (see www.uniprot.org, locate.imb.uq.edu.au, and www.ncbi.nlm.nih.gov/pubmed). CCP: Clathrin-coated pits; L: Lysosomes; LE: Late endosomes; MVB: Multivesicular bodies; SE: Sorting endosomes.

FIG. 11 shows the immunohistological identification of nanoparticle subcellular localization. Confocal microscopy confirmed distribution of RTV-NP within endocytic compartments (FIGS. 11A-H). Pearson's colocalization coefficients indicate RTV-NPs are preferentially distributed to Rab11 and Rab14 recycling endosomes compared with early endosomes, Rab8 or Rab7 endosomes, and lysosomes (FIG. 11I). Analysis of distribution of RTV-NP within acidified (degrading) compartments, identified by pHrodo™-dextran beads, revealed minimal overlap indicating RTV-NP likely bypass degradation within the cell and are primarily recycled for release. High RTV-NP colocalization with transferrin also indicates that particles are most likely recycled. Measure bars equal 1 μm. Graphical data represent the mean±standard error of the mean for n=3.

FIG. 12 shows the validation of nanoparticle subcellular localization. Disruption of endocytic recycling with siRNA (Rab8, 11 and 14) as well as disruption of cell secretion with brefeldin A resulted in knockout of the associated protein and caused RTV-NPs to be redistributed within monocyte-derived macrophages (FIGS. 12A and 12B). In each case, siRNA treatment resulted in aggregation of RTV-NPs at the perinuclear region within large vacuoles. siRNA silencing of specific proteins was confirmed by Western blot (FIG. 12C). High-performance liquid chromatography quantitation of RTV-NP in cells (FIG. 12D) and culture fluids (FIG. 12E) demonstrated that disruption of endocytic recycling and inhibition of secretion significantly increased cellular retention of RTV-NPs and reduced release. Upper p-value signifies difference from control cells and lower p-value signifies difference from cells treated with scrambled siRNA. Measure bars equal 1 μm. Graphical data represent the mean±standard error of the mean for n=3.

FIG. 13 shows ritonavir nanoparticles are transported during endocytic sorting. Since RTV-NPs were labeled with lipophillic dyes (DiD or DiO), which bind to the polymer coat but not the drug crystal itself, it was tested whether the endocytic distribution of drug matched that of labeled polymer. Treatment of MDM with RTV-NP and subsequent immune isolation of subcellular compartments and HPLC analysis of drug content (FIG. 13A). FIG. 13B provides an image of magnetic beads along with immune isolated endosomal compartments prior to HPLC analysis; the white matter on top of the bead pellet in the Rab11 tube was presumably RTV-NP filled endosomes. FIG. 13C provides HPLC analyses of immune isolated compartments confirmed a greater amount of RTV present in Rab11 endosomes than in either EEA1 or LAMP1. Graphical data represent the mean±standard error of the mean for n=3. ^{†}Significantly (p<0.01) different from control. Significantly (p<0.01) different from Rab11.

FIG. 14 shows ritovanir nanoparticles are released intact and retain their antiretroviral efficacy. Scanning electron microscopy (magnification 15,000×) of native RTV-NPs (FIG. 14A) and RTV-NPs released from cells into the surrounding medium (FIG. 14B). RTV-NPs were separated from dissolved drug by ultracentrifugation; the percentage of total drug in both particulate and dissolved form is shown. Total drug concentration was 40 μg/ml (FIG. 14C). Monocyte-derived macrophages were treated with either free RTV, native RTV-NP or released RTV-NP and subsequently challenged with HIV. Treatment of monocyte-derived macrophages with released RTV-NP reduced viral infection to similar levels as the native (non-endocytosed) particles as seen by p24 staining and formation of multinucleated giant cells (FIG. 14D), measurement of RT activity (FIG. 14E), and density of p24 staining (FIG. 14F). For both RT activity and p24 density measurements all data represent the mean±standard error of the mean for n=4.

FIG. 15 provides a schematic of possible intracellular pathways of ritonavir nanoparticles. RTV-NPs enter MDM via clathrin-coated pits and are then transported to the early endosome (EE) compartment. From the EE compartment, the particles can have three different fates: fast recycling via Rab4+ or 14+ endosomes; trafficking to late endosome, regulated in part by ESCRT machinery for eventual release as a secretory lysosome; or for most of the particles, transport to the recycling endosome (RE) compartment where they will be stored for long periods and slowly recycled via Rab11+ endosomes.

FIG. 16 provides a schematic of the synthesis of folate (FA) terminated poloxamers (P188 and P407).

FIG. 17 provides images of the morphology of ATV nanosuspensions. Scanning electron micrographs (SEM; 15,000× magnification) of ATV nanoformulations on top of a 0.2 μm polycarbonate membrane. ATV nanoformulations were all rod-shaped regardless of the type of polymer coating. Bar=1 micron.

FIG. 18 shows the uptake of ATV nanosuspensions containing unmodified P188 or FA-P188. FIG. 18A shows the uptake of ATV nanosuspensions was enhanced when particles were coated with 10% or 30% FA-P188 in unactivated human monocyte derived macrophages (MDM). FIG. 18B shows the uptake of ATV nanosuspensions was unchanged in MDM pre-treated with 50 ng/ml LPS for 24 hours. FIG. 18C shows the enhanced uptake of ATV nanosuspensions coated with 20% FA-P188 was reduced by addition of 2.5 mM free folic acid. Data are expresses as mean±SEM.

FIG. 19 shows the uptake of ATV nanosuspensions decorated with FA-P407. Uptake of P407-ATV nanosuspensions was enhanced by the inclusion of FA-P407 in the polymer coating. Data are expresses as mean SEM.

FIG. 20 shows macrophage uptake, retention and release of ATV nanosuspensions with and without folate-modified poloxamers. Uptake of ATV nanosuspensions containing P407 was enhanced over uptake of ATV nanosuspensions containing P188. Improved uptake for folate-conjugated versus unconjugated poloxamer-coated ATV nanosuspensions was observed. Cell retention profiles of ATV nanosuspensions through 15 days were similar for all polymer coatings and dependent on initial cell loading. Sustained ATV release into the medium was similar through 15 days for all formulations. Data are expressed as mean±SEM.

FIG. 21 shows the antiretroviral effects of ATV nanosuspensions. Reverse transcriptase (RT) activity in medium from cells loaded with ATV nanosuspensions for 8 hours and then challenged with HIV-1_{ADA} at 1, 5, 10, and 15 days after drug treatment. RT activity was measured by ³H-TTP incorporation. Data represent the average of N=8 measurements.

FIG. 22 shows the HIV-1 p24+ staining in MDM loaded with ATV nanosuspensions and infected with HIV-1_{ADA}. MDM were loaded with nanoART for 8 hours and then challenged with HIV-1 virus at 1, 5, 10, or 15 days after removal of ATV nanosuspensions from the culture medium. Measure bar±250 microns.

FIG. 23 provides a schematic of the synthesis of mannose terminated F127 (mannose-F127).

FIG. 24 shows the uptake of folate ATV nanoART in MDM. P188-FA, F127-FA, and F127-M represent the uptake of folate-F68 ATV nanoART, folate-F127 ATV nanoART, and mannose-F127 ATV nanoART in MDM, respectively. P188 and F127 represent the uptake of non-targeting F68 and F127 ATV nanoARTs in MDM.

### DETAILED DESCRIPTION OF THE INVENTION

Long-term antiretroviral therapy (ART) for human immunodeficiency virus type one (HIV-1) infection shows limitations in pharmacokinetics and biodistribution while inducing metabolic and cytotoxic aberrations. In turn, ART commonly requires complex dosing schedules and leads to the emergence of viral resistance and treatment failures. The nanoformulated ART compositions of the instant invention preclude such limitations and affect improved clinical outcomes. Herein, it is demonstrated that following clathrin-dependent endocytosis the nanoparticles (NPs) bypassed lysosomal degradation by sorting from early endosomes to recycling endosome pathways. Particles were released intact and retained complete antiretroviral efficacy. These results provide possible pathways of subcellular transport of antiretroviral nanoformulations that preserve both particle integrity and antiretroviral activities demonstrating the potent utility of this approach for targeted drug delivery. Indeed, the subcellular locale of the NPs and their slow release underlie long-term antiretroviral efficacy. In addition, the data demonstrates that cells such as macrophages can act as drug transporters and, importantly, neither degrade nor modify drug-laden particles in transit. As such, biologically active drug(s) are delivered unaltered to its intended target sites.

The instant invention encompasses nanoparticles for the delivery of compounds to a cell. In a particular embodiment, the nanoparticle is for the delivery of antiretroviral therapy to a subject. The nanoparticles of the instant invention comprise at least one compound of interest and at least one surfactant. These components of the nanoparticle, along with other optional components, are described hereinbelow.

### I. THERAPEUTIC AGENT

The nanoparticles of the instant invention may be used to deliver any agent(s) or compound(s), particularly bioactive agents (e.g., therapeutic agent or diagnostic agent) to a cell or a subject (including non-human animals). As used herein, the term “bioactive agent” also includes compounds to be screened as potential leads in the development of drugs or plant protecting agents. Bioactive agent and therapeutic agents include, without limitation, polypeptides, peptides, glycoproteins, nucleic acids, synthetic and natural drugs, peptoides, polyenes, macrocyles, glycosides, terpenes, terpenoids, aliphatic and aromatic compounds, small molecules, and their derivatives and salts. In a particular embodiment, the therapeutic agent is a chemical compound such as a synthetic and natural drug. While any type of compound may be delivered to a cell or subject by the compositions and methods of the instant invention, the following description of the inventions exemplifies the compound as a therapeutic agent.

The nanoparticles of the instant invention comprise at least one therapeutic agent. The nanoparticles are generally crystalline (solids having the characteristics of crystals) nanoparticles of the therapeutic agent, wherein the nanoparticles typically comprise about 99% pure therapeutic agent. In a particular embodiment, the nanoparticles are synthesized by adding the therapeutic agent, particularly the free base form of the therapeutic agent, to a surfactant (described below) solution and then generating the nanoparticles by wet milling or high pressure homogenization. The therapeutic agent and surfactant solution may be agitated prior the wet milling or high pressure homogenization.

In a particular embodiment, the resultant nanoparticle is up to 1 μm in diameter. In a particular embodiment, the nanoparticle is about 200 nm to about 500 nm in diameter, particularly about 250-350 nm in diameter. In a particular embodiment, the nanoparticles are rod shaped, particularly elongated rods, rather than irregular or round shaped. The nanoparticles of the instant invention may be neutral or charged. The nanoparticles may be charged positively or negatively.

The therapeutic agent may be hydrophobic, a water insoluble compound, or a poorly water soluble compound. For example, the therapeutic agent may have a solubility of less than about 10 mg/ml, less than 1 mg/ml, more particularly less than about 100 μg/ml, and more particularly less than about 25 μg/ml in water or aqueous media in a pH range of 0-14, particularly between pH 4 and 10, particularly at 20° C.

In a particular embodiment, the therapeutic agent of the nanoparticles of the instant invention is an antimicrobial. In another embodiment, the therapeutic agent is an antiviral, more particularly an antiretroviral. The antiretroviral may be effective against or specific to lentiviruses. Lentiviruses include, without limitation, human immunodeficiency virus (HIV) (e.g., HIV-1, HIV-2), bovine immunodeficiency virus (BIV), feline immunodeficiency virus (FIV), simian immunodeficiency virus (SIV), and equine infectious anemia virus (EIA). In a particular embodiment, the therapeutic agent is an anti-HIV agent.

An anti-HIV compound or an anti-HIV agent is a compound which inhibits HIV. Examples of an anti-HIV agent include, without limitation:

(I) Nucleoside-analog reverse transcriptase inhibitors (NRTIs). NRTIs refer to nucleosides and nucleotides and analogues thereof that inhibit the activity of HIV-1 reverse transcriptase. An example of nucleoside-analog reverse transcriptase inhibitors is, without limitation, adefovir dipivoxil.

(II) Non-nucleoside reverse transcriptase inhibitors (NNRTIs). NNRTIs are allosteric inhibitors which bind reversibly at a nonsubstrate-binding site on the HIV reverse transcriptase, thereby altering the shape of the active site or blocking polymerase activity. Examples of NNRTIs include, without limitation, delavirdine (BHAP, U-90152; RESCRIPTOR®), efavirenz (DMP-266, SUSTIVA®), nevirapine (VIRAMUNE®), PNU-142721, capravirine (S-1153, AG-1549), emivirine (+)-calanolide A (NSC-675451) and B, etravirine (TMC-125), rilpivirne (TMC278, Edurant™), DAPY (TMC120), BILR-355 BS, PHI-236, and PHI-443 (TMC-278).

(III) Protease inhibitors (PI). Protease inhibitors are inhibitors of the HIV-1 protease. Examples of protease inhibitors include, without limitation, darunavir, amprenavir (141W94, AGENERASE®), tipranivir (PNU-140690, APTIVUS®), indinavir (MK-639; CRIXIVAN®), saquinavir (INVIRASE®, FORTOVASE®), fosamprenavir (LEXIVA®), lopinavir (ABT-378), ritonavir (ABT-538, NORVIR®), atazanavir (REYATAZ®), nelfinavir (AG-1343, VIRACEPT®), lasinavir (BMS-234475/CGP-61755), BMS-2322623, GW-640385X (VX-385), AG-001859, and SM-309515.

(IV) Fusion inhibitors (FI). Fusion inhibitors are compounds, such as peptides, which act by binding to HIV envelope protein and blocking the structural changes necessary for the virus to fuse with the host cell. Examples of fusion inhibitors include, without limitation, maraviroc (Selzentry®, Celsentri), enfuvirtide (INN, FUZEON®), T-20 (DP-178, FUZEON®) and T-1249.

(V) Integrase inhibitors. Integrase inhibitors are a class of antiretroviral drug designed to block the action of integrase, a viral enzyme that inserts the viral genome into the DNA of the host cell. Examples of fusion inhibitors include, without limitation, raltegravir, elvitegravir, and MK-2048.

Anti-HIV compounds also include HIV vaccines such as, without limitation, ALVAC® HIV (vCP1521), AIDSVAX®B/E (gp120), and combinations thereof. Anti-HIV compounds also include HIV antibodies (e.g., antibodies against gp120 or gp41), particularly broadly neutralizing antibodies.

In a particular embodiment, the anti-HIV agent of the instant invention is a protease inhibitor, NNRTI, or NRTI. In a particular embodiment, the anti-HIV agent is selected from the group consisting of indinavir, ritonavir, atazanavir, and efavirenz. More than one anti-HIV agent may be used, particularly where the agents have different mechanisms of action (as outlined above). In a particular embodiment, the anti-HIV therapy is highly active antiretroviral therapy (HAART).

### II. SURFACTANTS

As stated hereinabove, the nanoparticles of the instant invention comprise at least one surfactant. A “surfactant” refers to a surface-active agent, including substances commonly referred to as wetting agents, detergents, dispersing agents, or emulsifying agents. Surfactants are usually organic compounds that are amphiphilic. In a particular embodiment, the surfactant is an amphiphilic block copolymer. In a particular, embodiment, at least one surfactant of the nanoparticle is an amphiphilic block copolymer, particularly a copolymer comprising at least one block of poly(oxyethylene) and at least one block of poly(oxypropylene).

In a particular embodiment of the invention, the surfactant is present in the nanoparticle and/or surfactant solution to synthesize the nanoparticle (as described hereinabove) at a concentration ranging from about 0.0001% to about 5%. In a particular embodiment, the concentration of the surfactant ranges from about 0.1% to about 2%.

The surfactant of the instant invention may be charged or neutral. In a particular embodiment, the surfactant is positively or negatively charged, particularly negatively charged.

In a particular embodiment, the amphiphilic block copolymer is a copolymer comprising at least one block of poly(oxyethylene) and at least one block of poly(oxypropylene). Amphiphilic block copolymers are exemplified by the block copolymers having the formulas:

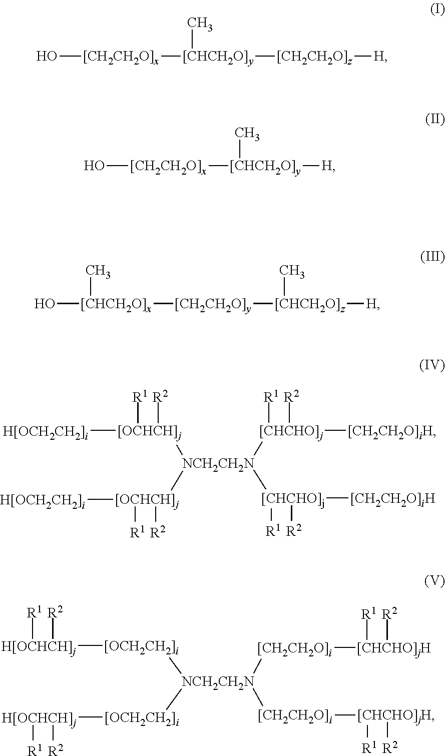

in which x, y, z, i, and j have values from about 2 to about 800, particularly from about 5 to about 200, more particularly from about 5 to about 80, and wherein for each R¹, R² pair, as shown in formula (IV) and (V), one is hydrogen and the other is a methyl group. The ordinarily skilled artisan will recognize that the values of x, y, and z will usually represent a statistical average and that the values of x and z are often, though not necessarily, the same. Formulas (I) through (III) are oversimplified in that, in practice, the orientation of the isopropylene radicals within the B block will be random. This random orientation is indicated in formulas (IV) and (V), which are more complete. Such poly(oxyethylene)-poly(oxypropylene) compounds have been described by Santon (Am. Perfumer Cosmet. (1958) 72(4):54-58); Schmolka (Loc. cit. (1967) 82(7):25-30), Schick, ed. (Non-ionic Surfactants, Dekker, N.Y., 1967 pp. 300-371). A number of such compounds are commercially available under such generic trade names as “lipoloxamers”, “Pluronics®,” “poloxamers,” and “synperonics.” Pluronic® copolymers within the B-A-B formula, as opposed to the A-B-A formula typical of Pluronics®, are often referred to as “reversed” Pluronics®, “Pluronic® R” or “meroxapol.” Generally, block copolymers can be described in terms of having hydrophilic “A” and hydrophobic “B” block segments. Thus, for example, a copolymer of the formula A-B-A is a triblock copolymer consisting of a hydrophilic block connected to a hydrophobic block connected to another hydrophilic block. The “polyoxamine” polymer of formula (IV) is available from BASF under the tradename Tetronic®. The order of the polyoxyethylene and polyoxypropylene blocks represented in formula (IV) can be reversed, creating Tetronic R®, also available from BASF (see, Schmolka, J. Am. Oil. Soc. (1979) 59:110).

Polyoxypropylene-polyoxyethylene block copolymers can also be designed with hydrophilic blocks comprising a random mix of ethylene oxide and propylene oxide repeating units. To maintain the hydrophilic character of the block, ethylene oxide can predominate. Similarly, the hydrophobic block can be a mixture of ethylene oxide and propylene oxide repeating units. Such block copolymers are available from BASF under the tradename Pluradot™. Poly(oxyethylene)-poly(oxypropylene) block units making up the first segment need not consist solely of ethylene oxide. Nor is it necessary that all of the B-type segment consist solely of propylene oxide units. Instead, in the simplest cases, for example, at least one of the monomers in segment A may be substituted with a side chain group.

A number of poloxamer copolymers are designed to meet the following formula:

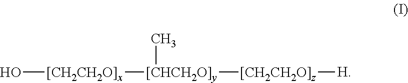

Examples of poloxamers include, without limitation, Pluronic® L31, L35, F38, L42, L43, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R2, 17R4, 17R8, 22R4, 25R1, 25R2, 25R4, 25R5, 25R8, 31R1, 31R2, and 31R4. Pluronic® block copolymers are designated by a letter prefix followed by a two or a three digit number. The letter prefixes (L, P, or F) refer to the physical form of each polymer, (liquid, paste, or flakeable solid). The numeric code defines the structural parameters of the block copolymer. The last digit of this code approximates the weight content of EO block in tens of weight percent (for example, 80% weight if the digit is 8, or 10% weight if the digit is 1). The remaining first one or two digits encode the molecular mass of the central PO block. To decipher the code, one should multiply the corresponding number by 300 to obtain the approximate molecular mass in daltons (Da). Therefore Pluronic nomenclature provides a convenient approach to estimate the characteristics of the block copolymer in the absence of reference literature. For example, the code ‘F127’ defines the block copolymer, which is a solid, has a PO block of 3600 Da (12×300) and 70% weight of EO. The precise molecular characteristics of each Pluronic® block copolymer can be obtained from the manufacturer.

Other biocompatible amphiphilic copolymers include those described in Gaucher et al. (J. Control Rel. (2005) 109:169-188. Examples of other polymers include, without limitation, poly(2-oxazoline) amphiphilic block copolymers, Polyethylene glycol-Polylactic acid (PEG-PLA), PEG-PLA-PEG, Polyethylene glycol-Poly(lactide-co-glycolide) (PEG-PLG), Polyethylene glycol-Poly(lactic-co-glycolic acid) (PEG-PLGA), Polyethylene glycol-Polycaprolactone (PEG-PCL), Polyethylene glycol-Polyaspartate (PEG-PAsp), Polyethylene glycol-Poly(glutamic acid) (PEG-PGlu), Polyethylene glycol-Poly(acrylic acid) (PEG-PAA), Polyethylene glycol-Poly(methacrylic acid) (PEG-PMA), Polyethylene glycol-poly(ethyleneimine) (PEG-PEI), Polyethylene glycol-Poly(L-lysine) (PEG-PLys), Polyethylene glycol-Poly(2-(N,N-dimethylamino)ethyl methacrylate) (PEG-PDMAEMA) and Polyethylene glycol-Chitosan derivatives.

In a particular embodiment, the surfactant comprises at least one selected from the group consisting of poloxamer 188, poloxamer 407, polyvinyl alcohol (PVA), 1,2-distearoyl-phosphatidyl ethanolamine-methyl-polyethyleneglycol conjugate-2000 (mPEG₂₀₀₀DSPE), sodium dodecyl sulfate (SDS), and 1,2-dioleoyloxy-3-trimethylammoniumpropane (DOTAP).

The surfactant of the instant invention may be linked to a targeting ligand. A targeting ligand is a compound that will specifically bind to a specific type of tissue or cell type. In a particular embodiment, the targeting ligand is a ligand for a cell surface marker/receptor. The targeting ligand may be an antibody or fragment thereof immunologically specific for a cell surface marker (e.g., protein or carbohydrate) preferentially or exclusively expressed on the targeted tissue or cell type. The targeting ligand may be linked directly to the surfactant or via a linker. Generally, the linker is a chemical moiety comprising a covalent bond or a chain of atoms that covalently attaches the ligand to the surfactant. The linker can be linked to any synthetically feasible position of the ligand and the surfactant. Exemplary linkers may comprise at least one optionally substituted; saturated or unsaturated; linear, branched or cyclic alkyl group or an optionally substituted aryl group. The linker may also be a polypeptide (e.g., from about 1 to about 10 amino acids, particularly about 1 to about 5). The linker may be non-degradable and may be a covalent bond or any other chemical structure which cannot be substantially cleaved or cleaved at all under physiological environments or conditions.

In a particular embodiment, the targeting ligand is a macrophage targeting ligand. Macrophage targeting ligands include, without limitation, folate receptor ligands (e.g., folate (folic acid) and folate receptor antibodies and fragments thereof (see, e.g., Sudimack et al. (2000) Adv. Drug Del. Rev., 41:147-162)), mannose receptor ligands (e.g., mannose), and formyl peptide receptor (FPR) ligands (e.g., N-formyl-Met-Leu-Phe (fMLF)). As demonstrated hereinbelow, the targeting of the nanoparticles to macrophage provides for central nervous system targeting (e.g., brain targeting), greater liver targeting, decreased excretion rates, decreased toxicity, and prolonged half life compared to free drug or non-targeted nanoparticles.

### III. ADMINISTRATION

The instant invention encompasses compositions comprising at least one nanoparticle of the instant invention (sometimes referred to herein as nanoART) and at least one pharmaceutically acceptable carrier. As stated hereinabove, the nanoparticle may comprise more than one therapeutic agent. In a particular embodiment, the composition comprises a first nanoparticle comprising a first therapeutic agent(s) and a second nanoparticle comprising a second therapeutic agent(s), wherein the first and second therapeutic agents are different. The compositions of the instant invention may further comprise other therapeutic agents (e.g., other anti-HIV compounds).

The present invention also encompasses methods for preventing, inhibiting, and/or treating microbial infections (e.g., viral or bacterial), particularly retroviral or lentiviral infections, particularly HIV infections (e.g., HIV-1). The pharmaceutical compositions of the instant invention can be administered to an animal, in particular a mammal, more particularly a human, in order to treat/inhibit an HIV infection. The pharmaceutical compositions of the instant invention may also comprise at least one other anti-microbial agent, particularly at least one other anti-HIV compound/agent. The additional anti-HIV compound may also be administered in separate composition from the anti-HIV NPs of the instant invention. The compositions may be administered at the same time or at different times (e.g., sequentially).

The dosage ranges for the administration of the compositions of the invention are those large enough to produce the desired effect (e.g., curing, relieving, treating, and/or preventing the HIV infection, the symptoms of it (e.g., AIDS, ARC), or the predisposition towards it). In a particular embodiment, lower doses of the composition of the instant invention are administered, e.g., about 50 mg/kg or less, about 25 mg/kg or less, or about 10 mg/kg or less. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The nanoparticles described herein will generally be administered to a patient as a pharmaceutical preparation. The term “patient” as used herein refers to human or animal subjects. These nanoparticles may be employed therapeutically, under the guidance of a physician. While the therapeutic agents are exemplified herein, any bioactive agent may be administered to a patient, e.g., a diagnostic or imaging agent.

The compositions comprising the nanoparticles of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the nanoparticles in the chosen medium may be varied and the medium may be chosen based on the desired route of administration of the pharmaceutical preparation. Except insofar as any conventional media or agent is incompatible with the nanoparticles to be administered, its use in the pharmaceutical preparation is contemplated.

The dose and dosage regimen of nanoparticles according to the invention that are suitable for administration to a particular patient may be determined by a physician considering the patient's age, sex, weight, general medical condition, and the specific condition for which the nanoparticles are being administered and the severity thereof. The physician may also take into account the route of administration, the pharmaceutical carrier, and the nanoparticle's biological activity.

Selection of a suitable pharmaceutical preparation will also depend upon the mode of administration chosen. For example, the nanoparticles of the invention may be administered by direct injection or intravenously. In this instance, a pharmaceutical preparation comprises the nanoparticle dispersed in a medium that is compatible with the site of injection.

Nanoparticles of the instant invention may be administered by any method. For example, the nanoparticles of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In a particular embodiment, the nanoparticles are administered intravenously or intraperitoneally. Pharmaceutical preparations for injection are known in the art. If injection is selected as a method for administering the nanoparticle, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a nanoparticle of the present invention as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, oral, direct injection, intracranial, and intravitreal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of nanoparticles may be determined by evaluating the toxicity of the molecules or cells in animal models. Various concentrations of nanoparticles in pharmaceutical preparations may be administered to mice, and the minimal and maximal dosages may be determined based on the beneficial results and side effects observed as a result of the treatment. Appropriate dosage unit may also be determined by assessing the efficacy of the nanoparticle treatment in combination with other standard drugs. The dosage units of nanoparticle may be determined individually or in combination with each treatment according to the effect detected.

The pharmaceutical preparation comprising the nanoparticles may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The instant invention encompasses methods of treating a disease/disorder comprising administering to a subject in need thereof a composition comprising a nanoparticle of the instant invention and, particularly, at least one pharmaceutically acceptable carrier. The instant invention also encompasses methods wherein the subject is treated via ex vivo therapy. In particular, the method comprises removing cells from the subject, exposing/contacting the cells in vitro to the nanoparticles of the instant invention, and returning the cells to the subject. In a particular embodiment, the cells comprise macrophage. Other methods of treating the disease or disorder may be combined with the methods of the instant invention may be co-administered with the compositions of the instant invention.

The instant also encompasses delivering the nanoparticle of the instant invention to a cell in vitro (e.g., in culture). The nanoparticle may be delivered to the cell in at least one carrier.

### IV. DEFINITION

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E.W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc. In a particular embodiment, the treatment of a retroviral infection results in at least an inhibition/reduction in the number of infected cells.

A “therapeutically effective amount” of a compound or a pharmaceutical composition refers to an amount effective to prevent, inhibit, treat, or lessen the symptoms of a particular disorder or disease. The treatment of a microbial infection (e.g., HIV infection) herein may refer to curing, relieving, and/or preventing the microbial infection, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “antimicrobials” as used herein indicates a substance that kills or inhibits the growth of microorganisms such as bacteria, fungi, viruses, or protozoans.

As used herein, the term “antiviral” refers to a substance that destroys a virus or suppresses replication (reproduction) of the virus.

As used herein, the term “highly active antiretroviral therapy” (HAART) refers to HIV therapy with various combinations of therapeutics such as nucleoside reverse transcriptase inhibitors, non-nucleoside reverse transcriptase inhibitors, HIV protease inhibitors, and fusion inhibitors.

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). As used herein, the term “hydrophilic” means the ability to dissolve in water.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

An “antibody” or “antibody molecule” is any immunoglobulin, including antibodies and fragments thereof (e.g., scFv), that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

The following examples provide illustrative methods of practicing the instant invention, and are not intended to limit the scope of the invention in any way.

### Example 1

There is a great need to attenuate viral replication in tissue sanctuaries, specifically the central nervous system (CNS) (Rao et al. (2009) Expert Opin. Drug Deliv., 6:771-784; Varatharajan et al. (2009) Antivir. Res., 82:A99-A109). One way to achieve such goals is through application of nanoformulated drug delivery approaches (Mallipeddi et al. (2010) Int. J. Nanomed., 5:533-547; Wong et al. (2010) Adv. Drug Deliv. Rev., 62:503-517; Nowacek et al. (2009) Nanomed., 4:557-574; das Neves et al. (2010) Adv. Drug Deliv. Rev., 62:458-477). Indeed, nanoformulated compounds positively affect the pharmacokinetics and pharmacodynamics of antiretroviral therapy (ART) while simultaneously reducing secondary cellular and tissue toxicities (Chirenje et al. (2010) Expert Rev. Anti-Infect. Ther., 8:1177-1186; Destache et al. (2009) BMC Infect. Dis., 9:198; Dou et al. (2006) Blood 108:2827-2835; Dou et al. (2009) J. Immunol., 183:661-669; Mahajan et al. (2010) Curr. HIV Res., 8:396-404; Parikh et al. (2009) J. Virol., 83:10358-10365; Yang et al. (2010) Bioorg. Med. Chem., 18:117-123). In addition, cell-mediated transport of nanoformulated drugs have shown promise for improving delivery of medications to diseased organs, particularly the central nervous system (Dou et al. (2009) J. Immunol., 183:661-669). The system is based on the capabilities of blood borne macrophages to uptake nanoformulated materials, store them in intracellular compartments, and cross blood vessel walls to deliver drugs to sites of active disease. In addition to their phagocytic, clearance, antigen presentation and secretory functions, macrophages also serve as viral sanctuaries, vehicles for viral transport, and as reservoirs for ongoing HIV-1 replication (Benaroch et al. (2010) Retrovirology 7:29; Kuroda et al. (2010) J. Leukoc. Biol., 87:569-573; Le Douce et al. (2010) Retrovirology 7:32; Persidsky et al. (2003) J. Leukoc. Biol., 74:691-701).

Drug delivery systems may utilize monocyte-macrophages for antiretroviral therapy (ART) delivery for HIV-1 infection (Dou et al. (2009) J. Immunol., 183:661-669; Dou et al. (2007) Virology 358:148-158; Nowacek et al. (2010) J. Neuroimmune Pharmacol., 5:592-601; Nowacek et al. (2009) Nanomedicine 4:903-917). Here, nanoformulated drugs are composed of antiretroviral drug crystals and include indinavir (IDV), ritonavir (RTV), atazanavir (ATV), and efavirenz (EFV). For each parental drug, large crystals may be fractioned into nanoparticles (NPs) by wet milling in the presence of surfactants. These micro- to nanoformulated antiretroviral drugs are referred to as “nanoART.” Macrophages may then be used to uptake nanoART and slowly release them for long periods of time. The structure and composition of nanoformulated drugs have important effects on stability, cellular interactions, efficacy and cytotoxicity (Caldorera-Moore et al. (2010) Expert Opin. Drug Deliv., 7:479-495; Doshi et al. (2010) J. R. Soc. Interface 7:S403-S410; Huang et al. (2010) Biomaterials 31:438-448; Zolnik et al. (2010) Endocrinology 151:458-465).

Herein, optimization of monocyte-derived macrophage (MDM) platforms for cell-based delivery of nanoART for therapeutic gains is performed by improving manufacture, characterization and pharmacodynamics. Wet milling was utilized in development because it was previously used to manufacture crystalline nanoparticles of poorly water-soluble drugs and can be scaled upwards for clinical use (Tanaka et al. (2009) Chem. Pharm. Bull. (Tokyo) 57:1050-1057; Hu et al. (2004) Drug Dev. Ind. Pharm., 30:233-245).

### Materials and Methods

Preparation and Characterization of nanoART

Ritonavir (RTV) (Shengda Pharmaceutical Co., Zhejiang, China) and efavirenz (EFV) (Hetero Labs LTD., Hyderabad, India) were obtained in free base form. The free bases of indinavir (IDV) sulfate (Longshem Co., Shanghai, China) and atazanavir (ATV) sulfate (Gyma Laboratories of America Inc., Westbury, N.Y.) were made using a 1N NaOH solution. The surfactants used in this study were: poloxamer-188 (P188; Sigma-Aldrich, Saint Louis, Mo.), polyvinyl alcohol (PVA) (Sigma-Aldrich, Saint Louis, Mo.), 1,2-distearoyl-phosphatidyl ethanolamine-methyl-polyethyleneglycol conjugate-2000 (mPEG₂₀₀₀DSPE) (Genzyme Pharmaceuticals LLC., Cambridge, Mass.), sodium dodecyl sulfate (SDS) (Bio-Rad Laboratories, Hercules, Calif.), and 1,2-dioleoyloxy-3-trimethylammoniumpropane (DOTAP) (Avanti Polar Lipids Inc., Alabaster, Ala.). For preparation of each nanosuspension, surfactants were suspended in 10 mM HEPES buffer solution (pH 7.8) in the following 5 combinations (weight/volume): (1) 0.5% P188 alone; (2) 0.5% PVA and 0.5% SDS; (3) 0.5% P188 and 0.5% SDS; (4) 0.3% P188 and 0.1% mPEG₂₀₀₀DSPE; and (5) 0.5% P188, 0.2% mPEG₂₀₀₀DSPE, and 0.1% DOTAP. Free base drug (ATV, EFV, IDV or RTV; 0.6% by weight) was then added to surfactant solutions. The suspension was agitated using an Ultra-turrax® T-18 rotor-stator mixer until a homogeneous dispersion formed. The mixture was then transferred to a NETZSCH MicroSeries Wet Mill (NETZSCH Premier Technologies, LLC, Exton, Pa.) along with 50 mL of 0.8 mm grinding media (zirconium ceramic beads). The sample was processed for 30 minutes to 1 hour at speeds ranging from 600 to 4320 rpm until desired particle size was achieved. For determination of particle size, polydispersity, and surface charge, 20 μl of the nanosuspension was diluted 50-fold with distilled/deionized water and analyzed by dynamic light scattering using a Malvern Zetasizer Nano Series Nano-ZS (Malvern Instruments Inc., Westborough, Mass.). After the desired size was achieved, samples were centrifuged and the resulting pellet resuspended in the respective surfactant solution along with 9.25% sucrose to adjust tonicity. The final drug concentration was determined using high performance liquid chromatography (HPLC).

### Human Monocyte Isolation and Cultivation

Human monocytes, obtained by leukapheresis from HIV-1 and hepatitis seronegative donors, were purified by counter-current centrifugal elutriation. Monocytes were cultivated in DMEM with 10% heat-inactivated pooled human serum, 1% glutamine, 50 μg/ml gentamicin, 10 μg/ml ciprofloxacin and 1000 U/ml recombinant human macrophage-colony stimulating factor at a concentration of 1×10⁶ cells/ml at 37° C. (Gendelman et al. (1988) J. Exp. Med., 167:1428-1441).

### Electron Microscopy

Cell samples were fixed with 3% glutaraldehyde in 0.1 M phosphate buffer (pH 7.4) and further fixed with 1% osmium tetroxide in 0.1 M phosphate buffer (pH 7.4) for 1 hour. The samples were then dehydrated in a graduated ethanol series and embedded in Epon 812 (Electron Microscopic Sciences, Fort Washington, Pa.) for scanning electron microscopy. For transmission electron microscopy, thin sections (80 nm) were stained with uranyl acetate and lead citrate and observed under a Hitachi H7500-I transmission electron microscope (Hitachi High Technologies America Inc., Schaumburg, Ill.).

### NanoART Uptake and Release

A modified version of a previously published method was used to study uptake and release of nanoART (Nowacek et al. (2009) Nanomedicine 4:903-917). After 7 days of differentiation, MDM were treated with 100 μM nanoART. Uptake of nanoART was assessed without medium change for 8 hours. Adherent MDM were washed with phosphate buffered saline (PBS) and collected by scraping into PBS. Cells were pelleted by centrifugation at 950×g for 10 minutes at 4° C. Cell pellets were briefly sonicated in 200 μl of methanol and centrifuged at 20,000×g for 10 minutes at 4° C. The methanol extract was stored at −80° C. To study cell retention and release of nanoART, MDM were exposed to 100 μM nanoART for 8 hours, washed 3 times with PBS, and fresh nanoART-free media was added. MDM were cultured for 15 days with half medium exchanges every other day. On days 1, 5, 10 and 15 post-nanoART treatment, MDM were collected as described for cell uptake. Both cell extracts and medium were stored at −80° C. until HPLC analysis as previously described (Nowacek et al. (2010) J. Neuroimmune Pharmacol., 5:592-601).

### Live Cell Microscopy

MDM were stained using Vybrant® DiO cell-labeling solution (Invitrogen Corp., Carlsbad, Calif.) and viable MDM were identified by green fluorescence. NPs were labeled with lissamine rhodamine B 1,2-dihexadecanoyl-sn-glycero-3-phosphoethanolamine, triethylammonium salt (rDHPE; Invitrogen Corp., Carlsbad, Calif.) by adding fluorescent phospholipid to the surfactant coating. rDHPE-labeled NPs exhibited a red fluorescence. Based on the amount of tracer added, the number of labeled phospholipid molecules represented a very small fraction of the total coating material and contributed minimally to the thickness of the phospholipid coating. This was confirmed by size measurements that showed no significant differences in the sizes of nanoART formulated with or without rDHPE phospholipid. No differences were detected in the uptake or release of drug formulated with the fluorescent phospholipid compared to unlabeled particles. Images were captured every 30 seconds using a Nikon TE2000-U (Nikon Instruments Inc., Melville, N.Y.) with swept-field confocal microscope, 488 nm (green) and 568 nm (red) laser excitations, and a 60× objective.

### NanoART Antiretroviral Activities

MDM were treated with 100 μM nanoART for 8 hours, washed to remove excess drug, and infected with HIV-1_{ADA} at a multiplicity of infection of 0.01 infectious viral particles/cell (Gendelman et al. (1988) J. Exp. Med., 167:1428-1441) on days 10 and 15 post-nanoART treatment. Following viral infection, cells were cultured for ten days with half media exchanges every other day. Medium samples were collected on day 10 for measurement of progeny virion production as assayed by reverse transcriptase (RT) activity (Kalter et al. (1991) J. Immunol., 146:298-306). Parallel analyses for expression of HIV-1 p24 antigen by infected cells were performed by immunostaining.

### Reverse Transcriptase Activity

Medium samples (10 μl) were mixed with 10 μl of a solution containing 100 mM Tris-HCl (pH 7.9), 300 mM KCl, 10 mM DTT, and 0.1% nonyl phenoxylpolyethoxyl ethanol-40 (NP-40). The reaction mixture was incubated at 37° C. for 15 minutes. At this time 25 μl of a solution containing 50 mM Tris-HCl (pH 7.9), 150 mM KCl, 5 mM DTT, 15 mM MgCl₂, 0.05% NP-40, 10 μg/ml poly(A), 0.250 U/ml oligo d(T) 12-18, and 10 μCi/ml ³H-TTP was added to each well and incubated at 37° C. for 18 hours. Following incubation, 50 μl of ice-cold 10% trichloroacetic acid (TCA) was added to each well, the wells were harvested onto glass fiber filters, and the filters were assessed for ³H-TTP incorporation by β-scintillation spectroscopy (Kalter et al. (1991) J. Immunol., 146:298-306).

### Immunohistochemistry

Ten days after HIV-1 infection, cells were fixed with 4% phosphatebuffered paraformaldehyde for 15 minutes at roomtemperature (RT). Fixed cells were blocked with 10% BSA w/1% Triton X-100 (in PBS) for 30 minutes at RT and incubated with mouse monoclonal antibodies to HIV-1 p24 (1:100, Dako, Carpinteria, Calif.) for 3 hours. Binding of p24 antibody was detected using a Dako EnVision™+System-HRP labeled polymer antimouse secondary antibody and diaminobenzidine staining (Nowacek et al. (2010) J. Neuroimmune Pharmacol., 5:592-601; Nowacek et al. (2009) Nanomedicine 4:903-917). Cell nuclei were counterstained with hematoxylin. Images were taken using a Nikon TE300 microscope with a 40× objective.

### Cytotoxicity

To determine the effect of nanoART treatment on cell viability, MDM were treated with 100 μM nanoART for 8 hours, washed with PBS, and viability assessed using the MTT (3-(4,5-dimethylthiazol-2-yl)-2,5-diphenyltetrazolium bromide) assay. No effect on cell viability was observed for any of the formulations at the treatment concentrations used.

### NanoART Efficacy

Area under the curve (AUC) was determined over 8 h of MDM drug uptake, 15 days of MDM drug release (cell and medium levels), and 15 days of antiretroviral efficacy (RT activity in supernatant from HIV-infected MDM). Scoring of uptake and release was calculated as a decade-weighted ratio of each formulation as a function of the best AUC for each parental drug/experimental parameter. Scoring of antiretroviral activity was determined from the decade-weighted ratios of the inverse RT activity AUC for each parental drug. The AUC was determined from the level of each drug or RT activity as a function of time. Within each parental drug, the nanoformulation that yielded the highest AUC for uptake, cell retention, or release into the medium was scored as 10, while the formulation that yielded the lowest RT activity was scored as 10. The remainder of the formulations within each parental drug group was scored as a proportion to the best score of 10 based on the AUC/AUC_{best} ratio. The scores from each parameter for each drug nanoformulation were averaged to obtain the mean final score for each formulation. The formulations with mean final scores within the top 2 quartiles of each parental drug group were designated for continued testing (GO), while evaluations for those formulations with means within the lower 2 quartiles were discontinued (NOGO).

### Results

Manufacture and Characterization of nanoART

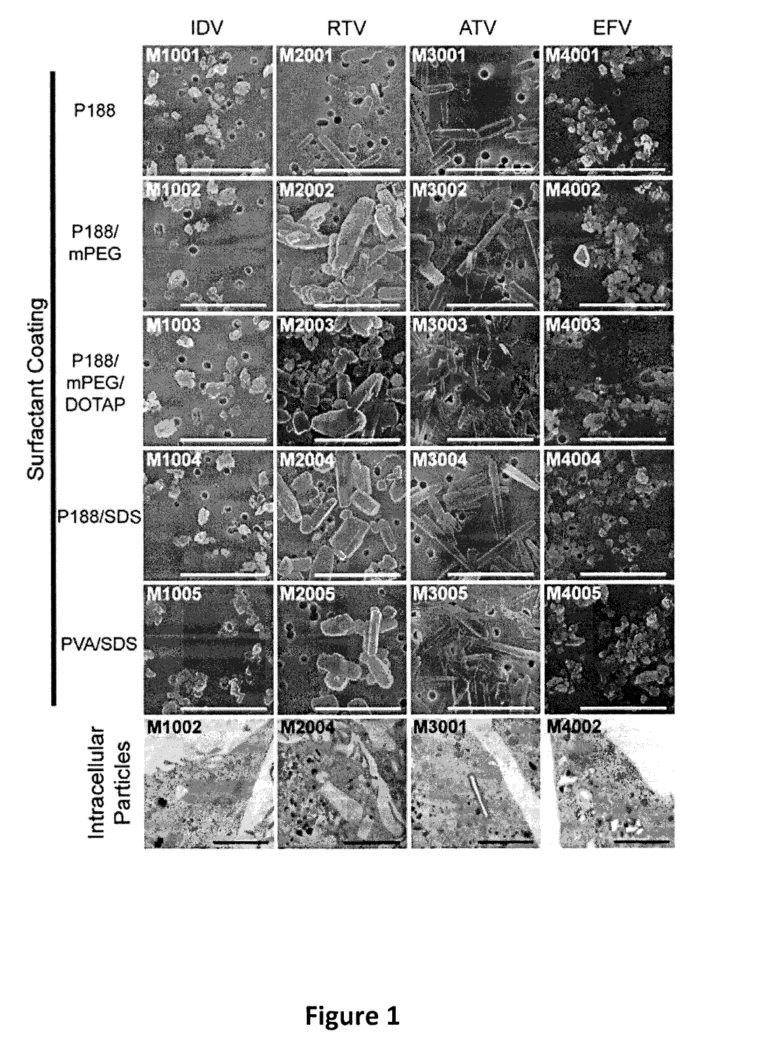

The 21 nanoART formulations consisted of nanosized drug crystals of free-base antiretroviral drugs coated with a thin layer of phospholipid surfactant. Five different surfactant combinations were used for each drug for a total of 5 formulations per drug. To determine the effect of size on cell uptake and release and on antiretroviral efficacy, an additional RTV formulation of larger particles was made using the surfactants P188/mPEG₂₀₀₀DSPE. All formulations were characterized based on their physical properties including coating, size, charge and shape. The formulations were of similar size and ranged from 233 nm (IDV formulation M1005) to 423 nm (RTV formulation M2005) with an average size of 309 nm (Table 1). Particle size distributions were not dissimilar to what is known for liposomal or other nanoformulated drug formulations manufactured via wet milling methods (Takatsuka et al. (2009) Chem. Pharm. Bull. (Tokyo) 57:1061-1067; Van Eerdenbrugh et al. (2007) Int. J. Pharm., 338:198-206). To estimate uniformity in particle size for each formulation, the polydispersity of each formulation was measured. The polydispersity indices (PDI) ranged from 0.180 (RTV formulation M2004) to 0.301 (ATV formulation M3004), indicating that while most of the particles were close to the calculated average size, there was a spectrum of sizes within each formulation. The additional RTV-P188/mPEG₂₀₀₀DSPE formulation (M2006) at a size of 540 nm was approximately twice the size of M2002 (265 nm). The zeta potential for each formulation was also determined. The most negatively charged formulations were those that contained P188 and SDS as the surfactants (M1004, M2004, M3004 and M4004). Addition of DOTAP imparted a positive charge to formulations M1003, M2003, M3003 and M4003. The remaining surfactant combinations gave the formulations varying degrees of negative charge. Particle morphology varied depending on drug; however, all formulations of the same drug were of similar shape (FIG. 1). IDV and EFV particles were polygonal-shaped with rough edges. ATV formulations resembled long thin rods with smooth edges, while RTV formulations resembled shorter and thicker rods, with smooth edges. Transmission electron microscopy confirmed intracellular inclusion of nanoART and demonstrated that the structural integrity of the nanoART is retained inside the cells.

TABLE 1
Physicochemical characteristics of nanoART.

| | Formu- | | Size | | Zeta Potential |
|---|---|---|---|---|---|
| Drug^{a} | lation | Surfactant | (nm)^{b} | PDI^{c} | (mV) |
| IDV | M1001 | P188 | 302 | 0.259 | −10.46 |
| | M1002 | P188, mPEG₂₀₀₀-DSPE | 332 | 0.269 | −35.63 |
| | M1003 | P188, mPEG₂₀₀₀-DSPE, | 340 | 0.264 | 21.64 |
| | | DOTAP | | | |
| | M1004 | P188, SDS | 252 | 0.286 | −40.58 |
| | M1005 | PVA, SDS | 233 | 0.273 | −7.47 |
| RTV | M2001 | P188 | 347 | 0.235 | −13.52 |
| | M2002 | P188, mPEG₂₀₀₀-DSPE | 265 | 0.258 | −23.69 |
| | M2003 | P188, mPEG₂₀₀₀-DSPE, | 375 | 0.272 | 21.65 |
| | | DOTAP | | | |
| | M2004 | P188, SDS | 360 | 0.18 | −36.70 |
| | M2005 | PVA, SDS | 423 | 0.21 | −31.96 |
| | M2006 | P188, mPEG₂₀₀₀-DSPE | 540 | 0.192 | −25.47 |
| ATV | M3001 | P188 | 281 | 0.288 | −15.31 |
| | M3002 | P188, mPEG₂₀₀₀-DSPE | 269 | 0.241 | −26.52 |
| | M3003 | P188, mPEG₂₀₀₀-DSPE, | 280 | 0.237 | 25.85 |
| | | DOTAP | | | |
| | M3004 | P188, SDS | 296 | 0.301 | −42.63 |
| | M3005 | PVA, SDS | 260 | 0.287 | −8.18 |
| EFV | M4001 | P188 | 311 | 0.273 | −13.60 |
| | M4002 | P188, mPEG₂₀₀₀-DSPE | 325 | 0.281 | −32.47 |
| | M4003 | P188, mPEG₂₀₀₀-DSPE, | 331 | 0.235 | 23.29 |
| | | DOTAP | | | |
| | M4004 | P188, SDS | 315 | 0.259 | −41.38 |
| | M4005 | PVA, SDS | 290 | 0.241 | −15.25 |

^{a}Abbreviations used in the table: ATV: atazanavir; DOTAP: (1-oleoyl-2-[6-[(7-nitro-2-1,3-benzoxadiazol-4-yl) amino]hexanoyl]-3-trimethylammonium propane); DSPE: 1,2-distearoyl-phosphatidyl-ethanolamine; EFV: efavirenz; IDV: indinavir; P188: poloxamer 188 (also termed Pluronic ™ F68); PVA: polyvinylalcohol; RTV: ritonavir; SDS: sodium dodecyl sulfate.
^{b}The particle sizes and
^{c}polydispersity indices (PDI) were determined by dynamic light scattering (DLS); the z-average diameters are presented. At least 4 iterations for each reading were taken and the readings varied by less than 2%.

### NanoART Uptake

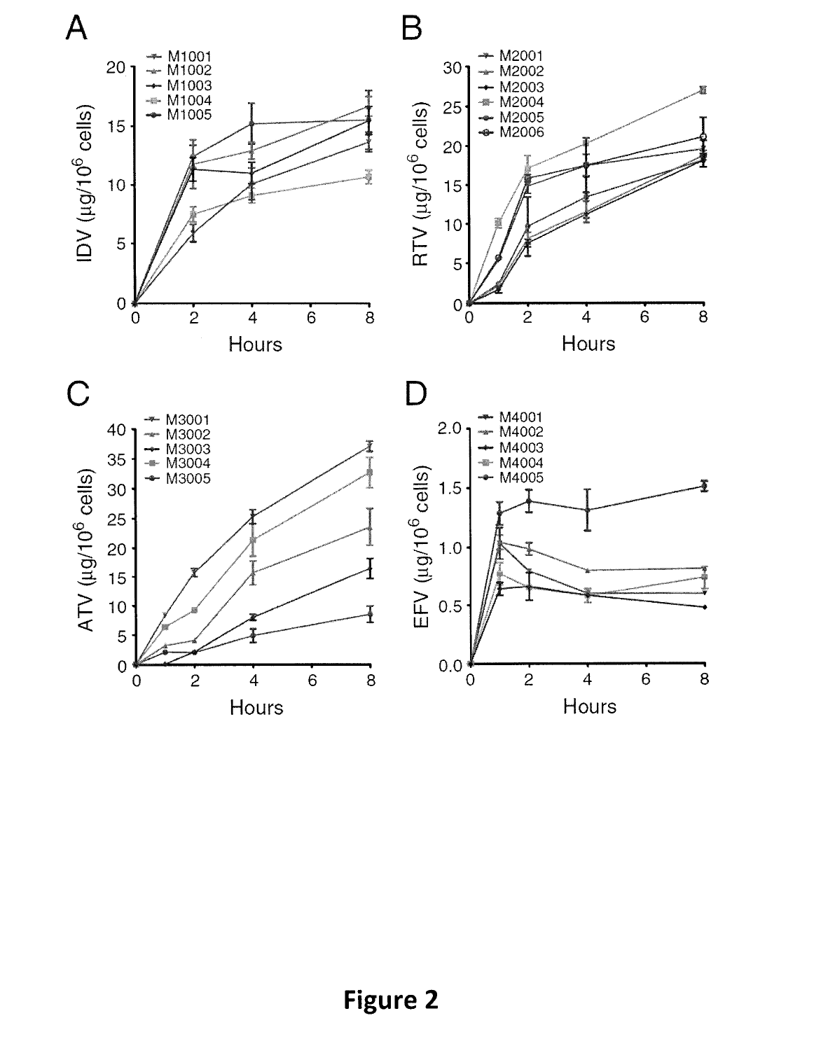

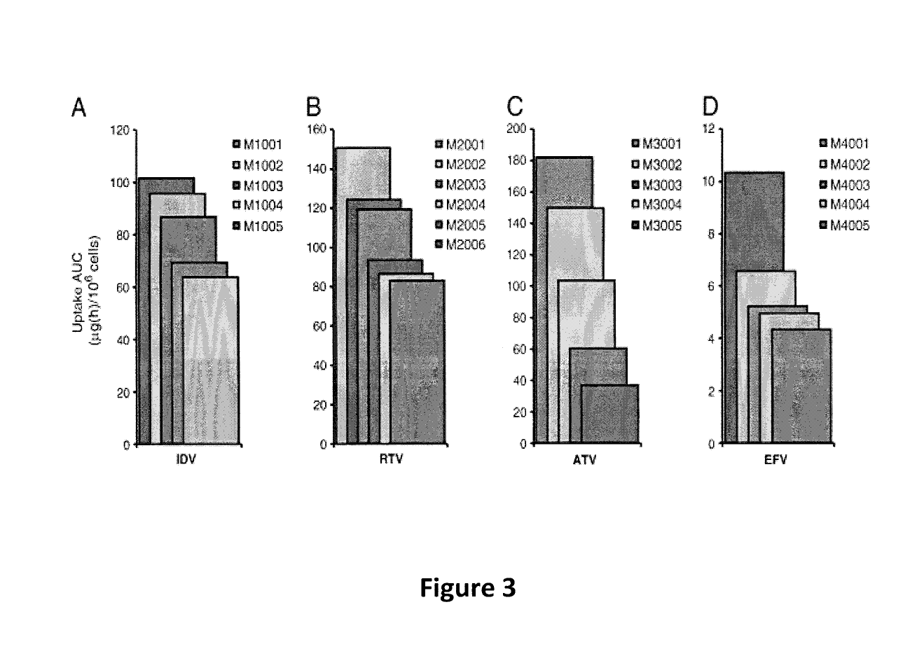

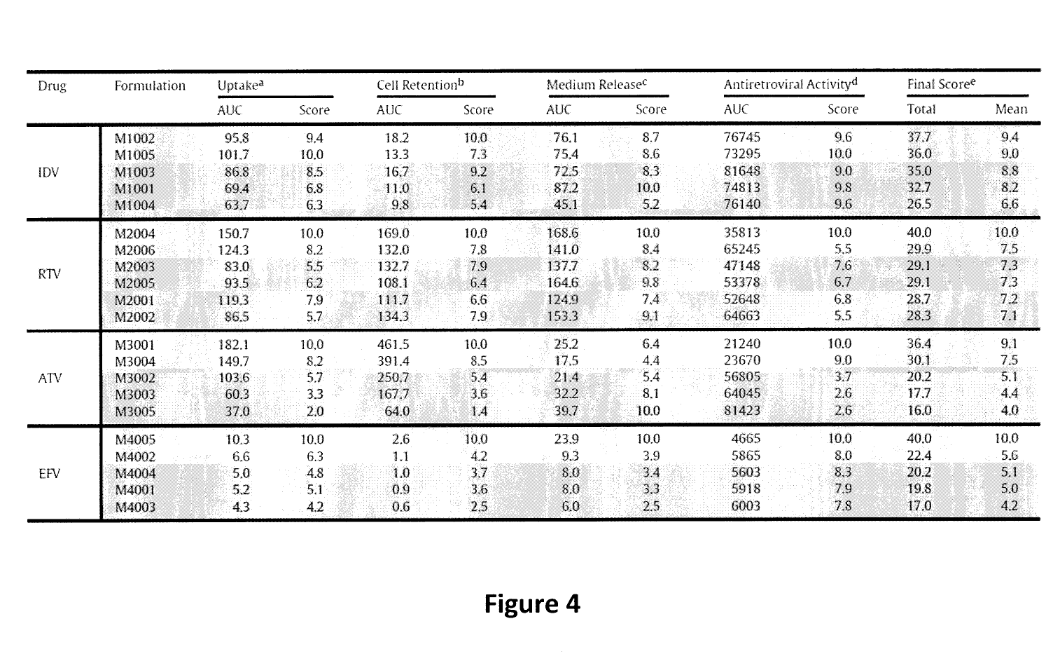

After characterizing their physical properties, the formulations were tested for in vitro PK and cellular handling by MDM. NanoART uptake in MDM showed that >95% of absolute uptake occurs by 8 hours for most nanoART (Dou et al. (2006) Blood 108:2827-2835; Dou et al. (2007) Virology 358:148-158; Nowacek et al. (2010) J. Neuroimmune Pharmacol., 5:592-601; Nowacek et al. (2009) Nanomedicine 4:903-917). Therefore, all uptake experiments were performed for 8 hours, and the amount of drug contained in the cells at that time was considered the maximum. For all IDV formulations, the rate of uptake was similar, and 85% of maximum uptake occurred by 4 hours (FIG. 2A). At 8 hours cell drug levels ranged from 10.7 to 16.7 μg/10⁶ cells for M1004 and M1002, respectively. For all RTV formulations, the rate of uptake was also similar (FIG. 2B). By 4 hours about 80% of maximum uptake had occurred. Maximum levels of RTV in the cells at 8 h ranged from 18.2 to 27.0 μg/10⁶ cells for M2005 and M2004, respectively. In contrast to IDV and RTV, the rate of uptake differed among the ATV formulations and cell levels at 4 hours ranged from 65% to 90% of maximum (FIG. 2C). Maximum amount of nanoART uptake occurred for all ATV formulations at 8 hours. Maximum cell levels of ATV varied widely among the formulations, ranging from 8.6 to 37.1 μg/10⁶ cells for M3005 and M3001, respectively. For all EFV formulations, the rate of uptake was similar; and maximum uptake for most occurred by 1 hour (FIG. 2D). There was a narrow range of maximum cell levels for EFV formulations, from 0.5 to 1.5 μg/10⁶ cells for M4003 and M4005, respectively. Drug uptake was visualized in real-time using live cell confocal imaging. In these experiments green-labeled MDM were treated with red-labeled M3001 or M3005, and an image was taken every 30 seconds for 4 hours. The resulting videos support the HPLC measurements of drug uptake. MDM accumulated M3001 particles at a much faster rate and in greater amounts than M3005 particles, as indicated by the number of red NP in the cytoplasm. FIG. 3 illustrates the AUC for drug concentrations in MDM over 8 hours of incubation. AUCs (total drug concentrations measured in μg/10⁶ cells) were evaluated for all nanoART formulations. These values were used for nanoART formulation scoring of uptake in FIG. 4.

### NanoART Cellular Retention, and Release

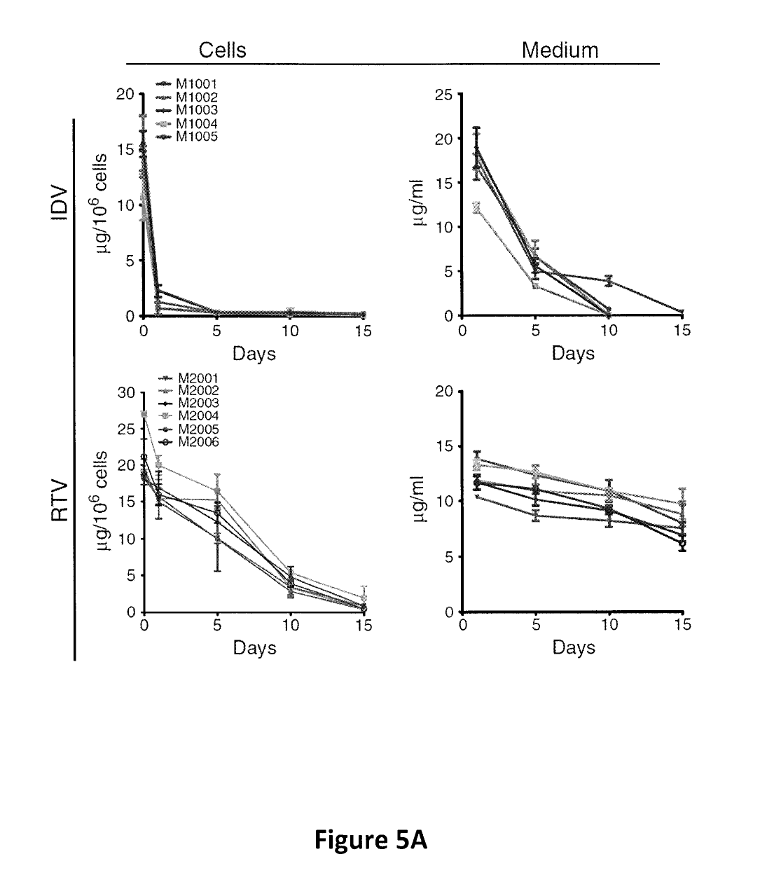

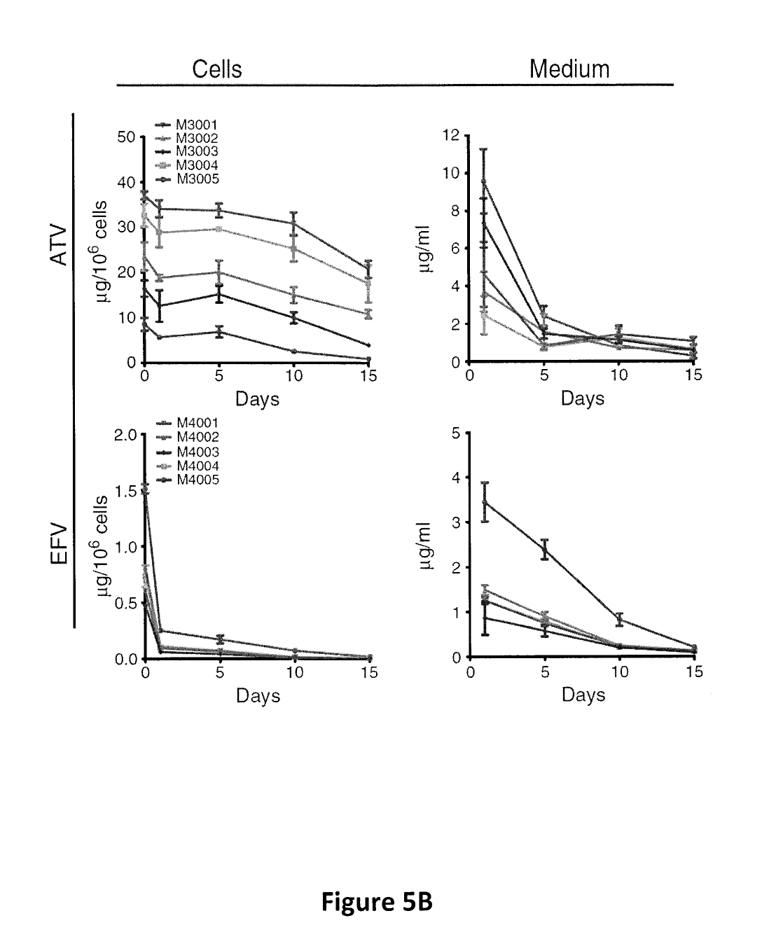

After an 8-hour loading period with the nanoART, MDM were cultured for another 15 days in drug-free medium to study both cellular retention of nanoART and release of drug into the media. Half-media exchanges occurred every other day over the 15 day period to facilitate release of the drug. For all IDV formulations, the profiles for cellular retention and cell release were similar (FIG. 5). Approximately 90% of what was contained within the cells after loading was released within the first 24 hours; however, for all IDV formulations, drug levels were low, but detectable within cells through day 15 (Table 2). IDV concentration within the media followed a steady decline from day 1 to day 10 (FIG. 5). For M1001, low but detectable amounts of drug were found in the medium through day 15; however, for all other IDV formulations, IDV was undetectable in the medium by day 15. For all RTV formulations, the profiles for cellular retention and cell release were also similar (FIG. 5). In contrast to IDV, only 20% of RTV contained within the cells after loading was released within the first 24 hours, and drug was still detectable within cells for all RTV formulations on day 15 (Table 2). RTV concentration in the medium declined steadily from day 1 to day 15, with levels still exceeding 6 μg/ml on day 15 for all RTV formulations. As for the IDV and RTV formulations, the profiles for cell retention were similar for all ATV formulations (FIG. 5); however, the absolute amount of drug varied depending on the loading level at 8 hours. Within the first 24 hours, approximately 4 μg/10⁶ cells of ATV were released for all formulations regardless of the initial cell levels following loading. However, after this initial burst of drug release, the cells retained the drug and only small amounts of were released over time. After 15 days, cells loaded with M3001 and M3004 still retained greater than 50% of the initial amount of drug (Table 2). The profiles of drug levels in media following release of ATV from nanoART-laden cells were again nearly identical for all formulations (FIG. 5). By day 5, the content of ATV within the media was greater than 1.5 μg/ml for all formulations except M3005 and remained between 0.25 and 1.1 μg/ml through day 15. For all EFV formulations, the profiles and amounts for both cellular retention and release were also similar (FIG. 5). As observed for IDV formulations, approximately 90% of EFV that was present within the cells at time zero was released within the first 24 hours; however on day 15, drug was still detectable within cells for all EFV formulations (Table 2). EFV concentrations within the medium steadily declined from day 1 to day 15 (FIG. 5), with low levels of detectable drug through day 15 for all EFV formulations (Table 2).

TABLE 2
ART release.

| | | Cell Levels (μg/10⁶ cells) | | Medium Levels (μg/ml) | |
|---|---|---|---|---|---|
| Drug | Formulation | 5 days | 15 days | 5 days | 15 days |
| IDV | M1001 | 0.29 ± 0.01 ^{a} | 0.22 ± 0.01 | 4.99 ± 0.90 | 0.32 ± 0.18 |
| | M1002 | 0.34 ± 0.06 | 0.26 ± 0.02 | 6.65 ± 1.79 | n.d.^{b} |
| | M1003 | 0.32 ± 0.01 | 0.14 ± 0.01 | 5.60 ± 0.79 | n.d. |
| | M1004 | 0.30 ± 0.01 | 0.11 ± 0.05 | 3.28 ± 0.12 | n.d. |
| | M1005 | 0.30 ± 0.03 | 0.09 ± 0.11 | 6.66 ± 0.88 | n.d. |
| RTV | M2001 | 10.06 ± 0.70 | 0.88 ± 0.23 | 8.69 ± 0.48 | 7.52 ± 0.53 |
| | M2002 | 15.23 ± 1.34 | 0.48 ± 0.15 | 10.92 ± 0.24 | 8.79 ± 1.13 |
| | M2003 | 12.28 ± 0.15 | 0.89 ± 0.11 | 10.11 ± 0.52 | 6.92 ± 0.17 |
| | M2004 | 16.43 ± 2.32 | 1.96 ± 1.62 | 12.61 ± 0.55 | 9.70 ± 1.40 |
| | M2005 | 9.96 ± 4.46 | 0.42 ± 0.26 | 12.34 ± 0.85 | 8.00 ± 1.64 |
| | M2006 | 13.48 ± 1.36 | 0.52 ± 0.14 | 11.09 ± 0.13 | 6.16 ± 0.68 |
| ATV | M3001 | 33.72 ± 1.56 | 20.72 ± 1.89 | 0.85 ± 0.09 | 1.07 ± 0.20 |
| | M3002 | 20.00 ± 2.61 | 10.77 ± 0.92 | 1.58 ± 0.93 | 0.60 ± 0.12 |
| | M3003 | 15.18 ± 1.86 | 3.92 ± 0.10 | 1.47 ± 0.27 | 0.60 ± 0.10 |
| | M3004 | 29.57 ± 0.22 | 17.51 ± 4.15 | 0.77 ± 0.18 | 0.64 ± 0.05 |
| | M3005 | 6.87 ± 1.26 | 0.88 ± 0.55 | 2.40 ± 0.53 | 0.29 ± 0.16 |
| EFV | M4001 | 0.07 ± 0.02 | 0.003 ± 0.001 | 0.74 ± 0.07 | 0.13 ± 0.003 |
| | M4002 | 0.07 ± 0.002 | 0.003 ± 0.0008 | 0.90 ± 0.10 | 0.13 ± 0.01 |
| | M4003 | 0.04 ± 0.007 | 0.003 ± 0.001 | 0.58 ± 0.12 | 0.10 ± 0.02 |
| | M4004 | 0.06 ± 0.0004 | 0.003 ± 0.0005 | 0.78 ± 0.07 | 0.12 ± 0.01 |
| | M4005 | 0.17 ± 0.04 | 0.02 ± 0.0004 | 2.38 ± 0.22 | 0.20 ± 0.04 |

^{a} Data are expressed as mean ± SEM, N = 3.
^{b}n.d.: not detectable (limit of detection 0.025 μg/ml).

### Antiretroviral Efficacy

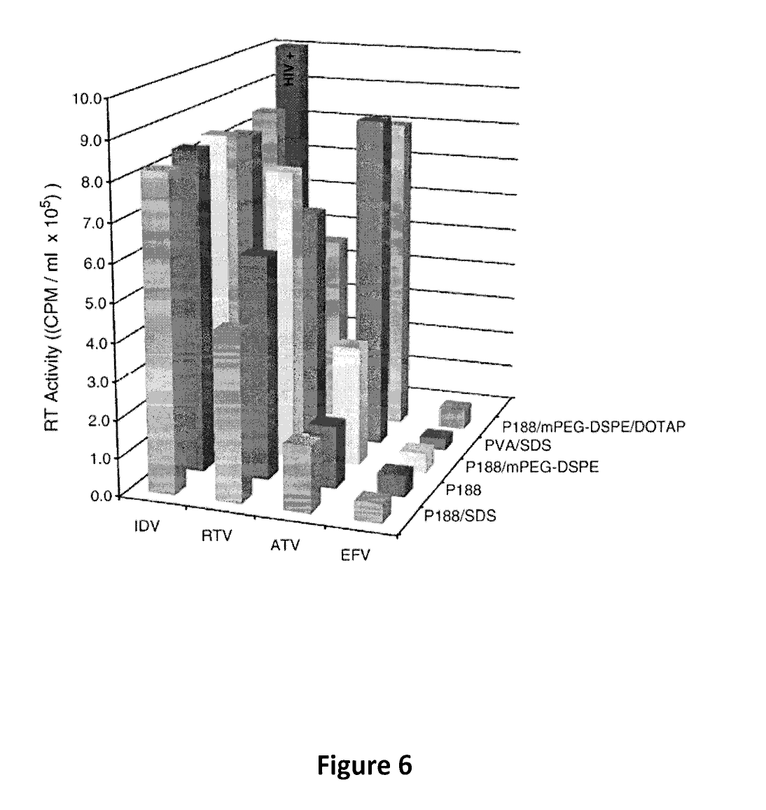

To determine the effectiveness of nanoART at inhibiting HIV replication, MDM were challenged with HIV-1_{ADA} at 1, 5, 10 and 15 days post-nanoART treatment. After HIV challenge, MDM continued to be cultured and media samples were collected 10 days later for RT analysis. All IDV formulations provided low, but similar antiretroviral efficacy. HIV replication was reduced by approximately 20% when viral challenge occurred on day 15 post-nanoART treatments (FIG. 6). In contrast, all EFV formulations provided nearly full protection against HIV infection through challenge day 15 post-nanoART treatment despite the relatively small amount of drug that remained within the cells. RTV and ATV formulations demonstrated wide spectrums of HIV inhibition. At viral challenge day 15, inhibition ranged from 25% to 60% for the RTV formulations (M2002 and M2004, respectively) and from 20% to 80% for the ATV formulations (M3005 and M3001, respectively). Of interest, RT activity directly correlated with amount of drug retained in the cells for ATV and EFV formulations, with a correlation coefficient of 0.92 for each drug group.

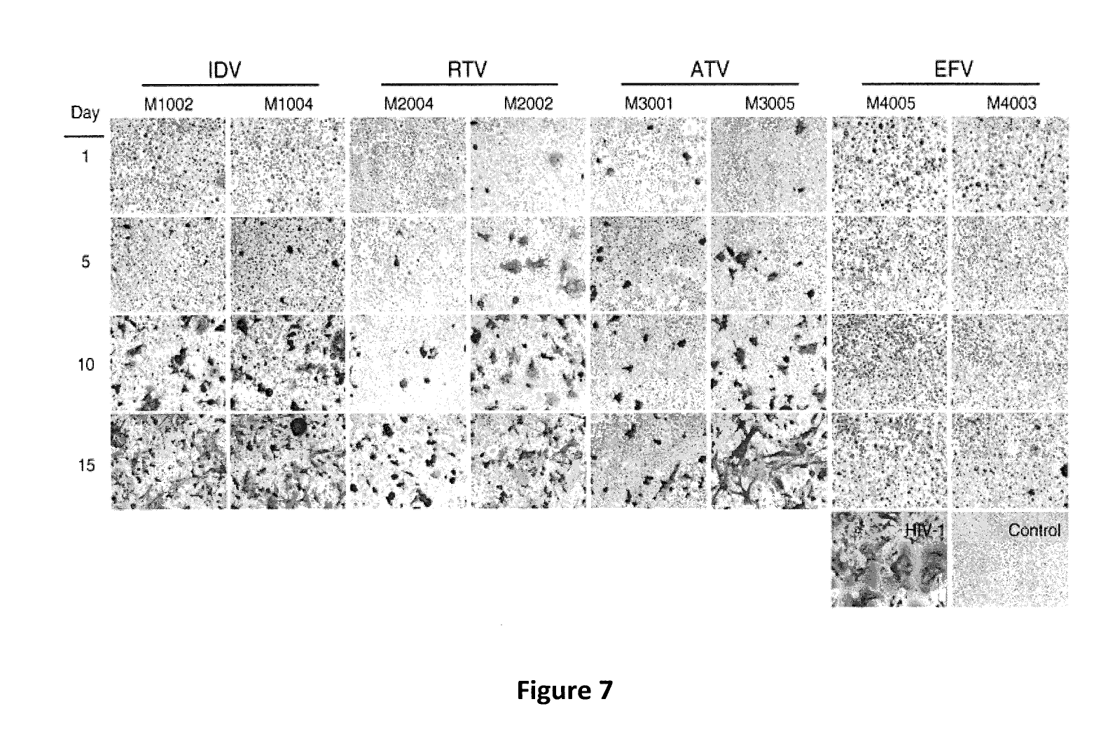

Expression of HIV-1 p24 antigen was used to verify RT activity and HIV proliferation. For each antiretroviral drug species, the best and worst performing formulations, as determined by uptake, cell retention, drug release and RT activity, were tested for comparison purposes. These formulations were M1004 and M1002 (IDV), M2004 and M2002 (RTV), M3001 and M3005 (ATV), and M4005 and M4003 (EFV). MDM loaded with nanoART were challenged with HIV-1ₘₘ on 1, 5, 10, and 15 days post-nanoART treatment and then tested for the presence of p24 antigen at 10 days post-infection. Empirical evaluation of p24 antigen expression demonstrated a gradual increase of HIV infection over time (indicated by increased brown staining) for all nanoART. However, the best performing formulation of each drug, i.e. M1004, M2004, M3001 and M4005, suppressed the increase in p24 expression to a greater extent than did the worst performing formulation, i.e. M1002, M2002, M3005 and M4003 (FIG. 7). Of particular interest, all EFV formulations suppressed viral infection out through challenge day 15. Expression of p24 for all nanoART formulations reflected the level of RT activity.

Scoring System for nanoART

All nanoformulations were evaluated for uptake into and release from MDM, as well as for their anti-retroviral activity in HIV-1-infected MDM. Nanoformulations within each experimental parameter were scored and ranked based on the best performing formulation within each parental drug group (FIG. 4). Data were ranked based on accumulated scores (Total) and mean final scores. A “Go” decision was given to formulations scoring within the top 2 median quartiles, while a “No Go” designation was given to those scoring in the bottom 2 median quartiles. However, the designation of “no go” does not in any way indicate that those formulations cannot or should not be used for therapeutic or other purposes, the designation only indicates that other formulations were more preferred based on the instant assays and results. IDV formulations M1002 and M1005 had the highest mean final scores and thus were given a “Go” decision. For the RTV formulations, the shared mean scores by M2003 and M2005 (7.3) were also the median; thus, only two formulations (M2004 and M2006) were given a “Go” designation. For ATV formulations, M3001 and M3002 were designated “Go.” A clear separation in score, 7.5 vs. 5.1, was observed between the “Go/No Go” ATV formulations. One EFV formulation, M4005, scored the highest for each parameter tested and had a final mean score of 10. The next highest final score for EFV formulations (M4002) was nearly half at 5.1 (M4005). Although the difference in mean final score was substantial for these two formulations, both were given the “Go” decision.

21 nanoART formulations of 4 antiretroviral drugs were manufactured, characterized and tested to assess nanoART in an MDM in vitro testing system. Drug type, surfactant coating, and shape demonstrated substantive effects on particle uptake, drug release, and antiretroviral responses while those that exerted minor effects were particle charge and size. Surfactant coating varied substantively between drug types. For IDV, RTV and EFV four of the five formulations tested similarly. The surfactant combination P188/mPEG₂₀₀₀DSPE was designated as a “GO” formulation for all drugs tested with the exception of ATV.

Particle shape had an impact on nanoART performance. IDV and EFV particles were rounded with irregular edges and showed diminished cell uptake. In contrast, the RTV and ATV were rod-like in shape, with smooth, regular edges. RTV rods were shorter with smoother corners, while ATV were longer rods with sharper edges. The most effective particle uptake was seen with M3001, an ATV formulation, suggesting that longer rods are taken up most rapidly. These results are consistent with studies that examined the effect of particle shape on phagocytosis kinetics in macrophages and found that spherical particles were taken up more slowly than short rods and that long rods were taken up more rapidly than short rods (Huang et al. (2010) Biomaterials 31:438-448; Chithrani et al. (2006) Nano Lett., 6:662-668; Gratton et al. (2008) Proc. Natl. Acad. Sci., 105:11613-11618).

One of the most important factors that affected nanoART performance was the chemical nature of the parental drug. All ATV formulations demonstrated good PK but relatively poor antiretroviral efficacy. Furthermore, despite the low uptake of EFV nanoART, they exhibited the best antiretroviral efficacies. Of interest, the solubility of free-base ATV is over 300 times greater than that for the other free-base drugs (ATV: 4-5 mg/ml versus IDV: 15 μg/ml, EFV: 9 μg/ml, or RTV: 1-2 μg/ml) (Wishart et al. (2008) Nucleic Acids Res., 36:D901-D906; Wishart et al. (2006) Nucleic Acids Res., 34:D668-D672) and uptake and release of the ATV nanoformulations appeared to be most-influenced by surfactant coating. NanoART may consist of up to 99% pure drug crystal and as a result, particular antiretroviral drugs may be better suited for MDM cell-mediated delivery than others. When comparing the antiretroviral activity of all nanoART formulations within a single drug group, a good predictor of efficacy is how much drug is contained within the cells. For EFV and ATV nanoART formulations, a strong correlation (0.92) was established between how much drug was contained within the cells and the degree of protection against HIV infection. Cells that contained more drug were provided a greater level of protection, regardless of how much drug was present in the surrounding medium. At days 5 and 15, the amount of drug present in the medium for all drug formulations exceeded EC₅₀ levels for anti-HIV activity reported for a variety of HIV strains and host cell types (1.7-25 nM, EFV; 35-200 nM, RTV; 5-29 nM IDV; 2-5 nM ATV) (Robinson et al. (2000) Antimicrob. Agents Chemother., 44:2093-2099). Additionally, day 5 medium levels for all drugs were equivalent to therapeutic human plasma levels (1.8-4.1 μg/ml, EFV; 3.5-9.6 μg/ml RTV; 0.15-8.0 μg/ml IDV and 0.3-2.2 μg/ml, ATV (Shannon et al., Haddad and Winchester's Clinical Management of Poisoning and Drug Overdose, 4th ed. Saunders Elsevier, Philadelphia, Pa., 2007; von Hentig et al. (2008) J. Antimicrob. Chemother., 62:579-582). Together these results indicate that nanoART primarily exert their antiretroviral effects inside the cell.

While the amount of nanoART contained within MDM is an important indicator of the degree of protection against HIV-1 infection, it is not the sole determinant. Some of the nanoART drugs were highly efficacious in very small amounts, while others that were present in cells at larger amounts were less efficacious. For example, on day 15, levels of IDV in nanoART treated cells were undetectable; yet, HIV-1 infection was still reduced by approximately 20%. In contrast, the amount of EFV, contained in cells after nanoART treatment was extremely low for all formulations, however, the cells were almost completely protected from HIV infection. In addition, ATV nanoART-treated cells had drug levels more than 1000 times that of EFV nanoART-treated cells, but were still infected with HIV to varying degrees. A possible explanation for this phenomenon is that not all nanoART traffic through the cell in an identical manner and may be stored in different subcellular compartments. If true, this would suggest that location of nanoART within the cell could be as important as how much drug actually enters the cell. For example, if nanoART is co-localized to the same endosomal compartment in which HIV replication is occurring, it may take only a small amount of drug to totally inhibit viral replication. On the other hand, nanoART stored in a separate compartment from where HIV replication is occurring, may be less efficacious even if present in larger amounts. The importance of internal mechanisms, intracellular trafficking, and sub-cellular storage of nanomaterials on their biologic effects has been demonstrated (Jiang et al. (2008) Nat. Nanotechnol., 3:145-150; Vallhov et al. (2007) Nano Lett., 7:3576-3582; Slowing et al. (2006) J. Am. Chem. Soc., 128:14792-14793).

Here, the two factors that had relatively lesser effect upon nanoART performance were size and charge. Other studies have shown that nanoparticle size can greatly affect function, however, no obvious differences in nanoART performance could be seen in the current study based upon particle size alone (Jiang et al. (2008) Nat. Nanotechnol., 3:145-150; Vallhov et al. (2007) Nano Lett., 7:3576-3582; Ferrari, M. (2008) Nat. Nanotechnol., 3:131-132). This lack of size effect could be due to the similarity in sizes of the nanoART, which ranged from 233 nm to 423 nm and did not generally vary more than 100 nm. An exception was the comparison of overall performance of M2006 and M2002; both were coated with the same surfactant combination but they differed in size by approximately 2-fold. M2002, which performed the worst overall of the RTV nanoART formulations, was about half the size of M2006, which performed second best. This implies that larger nanoART particles may perform better than smaller ones and parallels other findings that suggested larger nanoART (closer to 1 μm in size) may be taken up more efficiently by MDM with extended drug release. Particle charge also had more limited effects on nanoART performance. Most of the particles had a strong negative charge (<−15.0 mV), a few had relatively weak charges (between −15 mV and 0 mV), and a few had strong positive charges (>20 mV). It has been shown that strongly charged NPs are taken up better than those with weak or neutral charges (Roser et al. (1998) Eur. J. Pharm. Biopharm., 46:255-263). The nanoART formulation that performed the best for each drug tested had a strong negative charge, while those with weak negative charges (≦−8.2) ranked in the bottom two. Positively charged particles tended to be ranked in the middle of their groups. This result strongly indicates charged nanoART perform better than those with a neutral charge.

Repackaging traditional ART medications into nanoART and using macrophages as transporters offers several advantages for treating HIV-1 infection including: (i) prolonged plasma drug concentrations; (ii) slow and steady drug release; (iii) targeted delivery of drug to sites of active infection; and (iv) reduced toxicity. Both in vitro and in vivo studies have demonstrated that loading macrophages with nanoART greatly improves biodistribution and efficacy of antiretroviral medications, while simultaneously reducing cytotoxicities. In fact, in vivo studies using crystalline antiretroviral NPs have shown therapeutic benefit and indicate that upon in vivo administration, these types of NPs are likely taken up by macrophages.

### Example 2

Crystalline antiretroviral nanoparticles (nanoART) substantively increase drug-dosing intervals, reduce drug concentrations for administration, facilitate drug access to viral sanctuaries, diminish untoward side effects and improve drug availability to infected individuals. The latter targets patients who show poor compliance, have limited oral drug absorption or have few opportunities to obtain needed medicines. Monocytes and monocyte-derived macrophages (MDMs) used for nanoART carriage possess superior stability, less toxicity and potent antiretroviral efficacy compared with unformulated drugs (Dou et al. (2006) Blood 108:2827-2835; Dou et al. (2007) Virology 358:148-158; Nowacek et al. (2009) Nanomed. 4:903-917). Indeed, nanoART-laden MDMs are able to cross biological barriers in response to cytokine signaling, deliver drug(s) directly to infected tissues and drastically reduce viral replication (Dou et al. (2009) J. Immunol., 183:661-669). Animal studies have supported the in vitro results and demonstrated that clinically relevant amounts of drug are present within both serum and tissues for up to 3 months after a single administration (Baert et al. (2009) Eur. J. Pharm. Biopharm., 72:502-508; Van't Klooster et al. (2010) Antimicrob. Agents Chemother., 54:2042-2050).

Here, the subcellular location of nanoART from the point of initial uptake to final release was studied. It was observed that following rapid clathrin-dependent internalization, drug particles undergo sorting into a recycling pathway and as such bypass degradation. Drug was released intact from MDMs and had no reduction in antiretroviral efficacy. Interestingly, particle trafficking routes parallel what has been observed for HIV endocytic sorting. Such parallels between HIV and nanoART subcellular endocytic locale provide additional benefit in restricting viral replication. Taken together, the findings indicate macrophage-mediated drug delivery as a therapeutic option for a more efficient and simplified drug regimen for HIV-infected people.

### Materials & Methods

### Antibodies & Reagents

Goat antibody (Ab) to Rab11 and Rab7, along with human siRNA to Rab8, Rab11 and Rab14, were purchased from Santa Cruz Biotechnology (CA, USA). SilenceMag siRNA delivery reagent and magnetic plates were purchased from Oz Biosciences (Marseille, France). Rabbit Ab to lysosome-associated membrane protein 1 (LAMP1) was purchased from Novus Biologicals (CO, USA). Rabbit Abs to early endosome antigen 1 (EEA1), clathrin, Rab8 and Rab14 were purchased from Cell Signaling Technologies (MA, USA). pHrhodo-dextran conjugate for phagocytosis, rhodamine phalloidin, phalloidin Alexa Fluor® 488 and 647, transferrin (Tfn) conjugated to Alexa Fluor® 594, anti-rabbit Alexa Fluor® 488, 594, 647, anti-mouse Alexa Fluor® 488, 594, 647, anti-goat Alexa Fluor® 488, ProLong® Gold anti-fading solution with 4′,6-diamidino-2-phenylindole (DAPI) were all purchased from Molecular Probes (OR, USA). Dynasore and indomethacin were purchased from Sigma-Aldrich (MO, USA).

### RTV-NP Manufacturing & Characterization

Ritonavir nanoparticles (RTV-NPs) were prepared by high-pressure homogenization using an Avestin C-5 homogenizer (Avestin, Inc., ON, Canada) as described previously (see above and Nowacek et al. (2009) Nanomed., 4:903-917). Surfactants used to coat the drug crystals included poloxamer 188 (P188; Spectrum Chemicals, CA, USA), 1,2-distearoyl-phosphatidyl ethanolaminemethyl-polyethyleneglycol 2000 (mPEG₂₀₀₀-DSPE) and 1,2-dioleoyl-3-trimethylammoniumpropane (DOTAP), purchased from Avanti Polar Lipids, Inc. (AL, USA). To coat the nano-sized drug crystals, each surfactant was made up of (weight/vol %) P188 (0.5%), mPEG₂₀₀₀-DSPE (0.2%) and DOTAP (0.1%). The nanosuspensions were formulated at a slightly alkaline pH of 7.8 using either 10 mM sodium phosphate or 10 mM HEPES as a buffer. Tonicity was adjusted with glycerin (2.25%) or sucrose (9.25%). Free base drug was added to the surfactant solution to make a concentration of approximately 2% [weight to volume ratio (%)]. The solution was mixed for 10 minutes using an Ultra-Turrax™ T-18 (IKA® Works Inc. [NC, USA]) rotor-stator mixer to reduce particle size. The suspension was homogenized at 20,000 psi for approximately 30 passes or until desired particle size was achieved. Size was measured using a HORIBA LA 920 light scattering instrument (HORIBA Instruments Inc., CA, USA). For determination of polydispersity and zeta potential, 0.1 ml of the suspension was diluted into 9.9 ml of 10 mM HEPES, pH 7.4, and analyzed by dynamic light scattering using a Malvern Zetasizer Nano Series (Malvern Instruments Inc., MA, USA). At least four iterations for each reading were taken and the readings varied by less than 2%. After the desired size was achieved, samples were centrifuged and the resulting pellet resuspended in the surfactant solution containing 9.25% sucrose to adjust tonicity. Particle size and shape were characterized by scanning electron microscopy as described below. RTV-NPs were fluorescently labeled using the Vybrant™ 1,1′dioctadecyl-3,3,3′,3′-tetramethylindodicarbocyanine perchlorate (DiO) cell-labeling solution (Ex: 484 nm; Em: 501 nm) or 3,3″-dioctadecyloxacarbocyanine perchlorate (DiD; Ex: 644 nm; Em: 665 nm; Invitrogen [CA, USA]). Particles were labeled by combining 1 ml of RTV-NP suspension with 5 μl of dye and mixing overnight. After centrifugation at 20,000×g, the particles were washed with 5% human serum containing Dulbecco's modified Eagle medium (DMEM) until all excess dye was removed (at least five washes). Final drug content of the formulations were determined by high-performance liquid chromatography (HPLC).

### Human Monocyte Isolation & Cultivation

Human monocytes were obtained by leukapheresis from HIV and hepatitis seronegative donors, and were purified by counter-current centrifugal elutriation following approval by the Institutional Review Board at the University of Nebraska Medical Center. Wright-stained cytospins were prepared and cell purity assayed by immunolabeling with anti-CD68 (clone KP-1). Monocytes were cultivated at a concentration of 1×10⁶ cells/ml at 37° C. in a humidified atmosphere (5% CO₂) in DMEM supplemented with 10% heat-inactivated pooled human serum, 1% glutamine, 50 μg/ml gentamicin, 10 μg/ml ciprofloxacin and 1000 U/ml recombinant human macrophage colony-stimulating factor (MCSF), a generous gift from Pfizer Inc. (MA, USA). To induce differentiation to macrophages, monocytes were cultured for 7 days in the presence of MCSF.

### RTV-NP Uptake & Release

Monocyte-derived macrophages (2×10⁶ per well) were cultured with RTV-NPs at 100 μM. Uptake of particles was assessed without medium change for 24 hours with cell collection occurring at indicated times points. Adherent MDMs were collected by washing three times with 1 ml of phosphate-buffered saline (PBS), followed by scraping cells into 1 ml PBS. Samples were centrifuged at 950×g for 10 minutes at 4° C. and the supernatant removed. Cell pellets were sonicated in 200 μl of methanol and centrifuged at 10,000×g for 10 minutes at 4° C. The methanol extracts were stored at −80° C. until HPLC analysis was performed. After an initial 12-hour exposure to RTV-NPs, drug release from MDMs with half media exchanges every other day was evaluated over a 2-week period. Media samples were saved along with replicate cells and stored at −80° C. until HPLC analysis could be performed. Methanol-extracted cell suspensions were centrifuged at 21,800×g at 4° C. for 10 minutes. Media samples were thawed and deproteinated by the addition of methanol. The samples were centrifuged at 21,800×g at 4° C. for 10 minutes; supernatants were evaporated to dryness under vacuum and resuspended in 75 μl of 100% methanol. Triplicate 20 μl samples of processed media or cells were assessed by HPLC using a YMC Pack Octyl C8 column (Waters Inc. [MA, USA]) with a C8 guard cartridge. Mobile phase consisting of 47% acetonitrile/53% 25 mM KH₂PO₄, pH 4.15, was pumped at 0.4 ml/min with UV/Vis detection at 212 nm. Cell and medium levels of RTV were determined by comparison of peak areas to those of a standard curve of free drug (0.025-100 μg/ml) made in methanol.

### Immunocytochemistry & Confocal Microscopy

For immunofluorescence staining, cells were washed three times with PBS and fixed with 4% paraformaldehyde (PFA) at room temperature for 30 minutes. Cells were treated with blocking/permeabilizing solution (0.1% Triton, 5% bovine serum albumin [BSA] in PBS) and quenched with 50 mM NH4Cl for 15 minutes. Cells were washed once with 0.1% Triton in PBS and sequentially incubated with primary and secondary Ab, at room temperature. For MDMs stained with multiple Abs, nonspecific cross binding of secondary Abs was tested prior to immunostaining. Use of secondary Abs originating or recognizing the same hosts was avoided. Slides were covered in ProLong Gold antifading reagent with DAPI and imaged using a 63× oil lens in a LSM 510 confocal microscope (Carl Zeiss Microimaging, Inc., NY, USA).

### Imaging of Recycling Compartments

Monocyte-derived macrophages grown in poly-d-lysine-coated chamber slides were depleted of human serum by incubation with serum-free DMEM for 3 hours. Cells were coincubated with 1 μM Alexa 594-Tfn and 100 μM DiO-labeled RTV-NPs for 4 hours. Noninternalized particulates were removed by three sequential washes with PBS. Cells were fixed with 4% PFA and imaged using the 63× oil lens of a LSM 510 confocal microscope (Carl Zeiss Microimaging, Inc.).

### Detection of Acidified Compartments

Monocyte-derived macrophages were exposed to pHrhodo™ conjugated to dextran beads and 100 μM DiO-labeled RTV-NPs at 37° C. for 4 hours. Noninternalized particulates were removed by washing three times in Hanks Balanced Salt Solution, pH 7.4, followed by fixation with 4% PFA and imaging. Fluorescence intensity of pHrhodo™ dye at different pH levels (3.0-8.5) was previously determined using a M5 Florescence Microplate Reader (Molecular Devices [CA, USA]).

### Inhibition of RTV-NP Uptake

Monocyte-derived macrophages were washed three times in PBS and incubated with serum-free medium for 30 minutes. Cells were then exposed to 100 μM dynasore, 100 μM indomethacin, and a combination of both for 30 minutes in serum-free medium or left untreated. Cells were washed once with serum-free media, and DiD-labeled 100 μM RTV-NPs reconstituted in serum-free medium was added together with fresh inhibitors to the MDMs for 3 hours at 37° C. Cells were washed three times in PBS and mechanically detached using cell lifters. Cells were fixed in 4% PFA for 30 minutes and analyzed for RTV-NP uptake by flow cytometry. Data was acquired on a FACSCalibur™ flow cytometer using CellQuest Software (BD Biosciences, CA, USA). Replicate experiments were performed for HPLC analyses of drug content.

### Electron Microscopy

Samples were fixed by 3% glutaraldehyde in 0.1 M phosphate buffer (pH 7.4) and were further fixed in 1% osmium tetroxide in 0.1 M phosphate buffer (pH 7.4) for 1 hour. Samples were dehydrated in a graduated ethanol series and embedded in Epon 812 (Electron Microscopic Sciences [PA, USA]) for scanning electron microscopy. Thin sections (80 nm) were stained with uranyl acetate and lead citrate and observed under a transmission electron microscope (Hitachi H7500-I; Hitachi High Technologies America Inc. [IL, USA]).

### Enrichment of Endocytic Compartments

For enrichment of RTV-NP-associated compartments, RTV-NPs were labeled with 0.01% Brilliant Blue R-250 dye (Thermo-Fisher Scientific, MA, USA) for 12 hours at room temperature. Excess dye was removed by five washes in PBS and five subsequent centrifugations at 20,000×g for 10 minutes. Then, 100 μM RTV-NPs were added to MDMs for 12 hours at 37° C. Cells were washed three times in PBS, and RTV-NP uptake was visualized using the bright field settings on a Nikon Eclipse TE300 microscope (Nikon Instruments, Inc. [NY, USA]). Cells were detached in homogenization buffer (100 mM sucrose, 10 mM imidazole solution, pH 7.4) followed by 15 strokes on a Dounce homogenizer. Cellular debris and nuclei were removed by centrifugation at 500×g for 10 minutes. Supernatant was mixed at equal ratios with 60% sucrose, 10 mM imidazole solution, pH 7.4, to adjust sucrose concentration to 30% followed by layering on a 60, 35, 20 and 10% sucrose gradient and centrifugation at 100,000×g at 4° C. for 1 hour. The interface between 10-20 and 35-60% sucrose bands containing enriched endocytic compartments (blue) were collected. Pellets were collected by centrifugation at 100,000×g at 4° C. for 30 minutes, and sucrose was removed by washing three times in PBS. Pellets were then processed for proteomics analysis as described below.

Immune isolation of endocytic compartments was performed as described previously, with some modifications (Basyuk et al. (2003) Dev. Cell 5:161-174). MDMs'(400×10⁶ cells) were treated with 100 μM RTV-NPs for 6 hours. Cells were washed three times in PBS to remove extracellular RTV-NPs and then scraped in homogenization buffer (10 mM HEPES-KOH, pH 7.2, 250 mM sucrose, 1 mM EDTA and 1 mM Mg(OAc)₂). Cells were then disrupted by 15 strokes in a dounce homogenizer. Nuclei and unbroken cells were removed by centrifugation at 400×g for 10 minutes at 4° C. Protein A/G paramagnetic beads (20 μl of slurry; Millipore) conjugated to EEA1, lysosome-associated LAMP1, and Rab11 antibodies (binding in 10% BSA in PBS for 12 hours at 4° C.) were incubated with the supernatants. Beads alone were also exposed to cell lysate to test for binding specificity. Following 24 hours incubation at 4° C., EEA1+, LAMP1+ and Rab11+ endocytic compartments were washed and collected on a magnetic separator (Invitrogen). The RTV-NP content of each compartment was determined by HPLC as described above.

### Proteomic & Mass Spectrometry Analyses

Endocytic compartments were solubilized in lysis buffer, pH 8.5 [30 mM TrisCl, 7 M urea, 2 M thiourea, 4% (w/v) 3-[(3-cholamidopropyl)dimethylammonio]-1-propanesulfonate, 20 mM dithiothreitol and 1× protease inhibitor cocktail (Sigma)] by pipetting. Proteins were precipitated using a 2D Clean up Kit and quantified by 2D Quant (GE Healthcare [WI, USA]) per the manufacturer's instructions. Samples were run on Bis-Tris 4-12% and 7% Tris-Glycine gels (Invitrogen) to separate low- and high-molecular-weight proteins. Electrophoresis was followed by fixation on 10% methanol, 7% acetic acid for 1 hour and Coomassie staining at room temperature for 24 hours. Bands were manually excised followed by in-gel tryptic digestion (10 ng/spot of trypsin [Promega, WI, USA]) for 16 hours at 37° C. Peptide extraction and purification using μC18 ZipTips® (Millipore, MA, USA) were performed on the Proprep™ Protein Digestion and Mass Spec Preparation Systems (Genomic Solutions, MI, USA).

Extracted peptides were fractionated on a microcapillary RP-C₁₈ column (New Objectives, Inc. [MA, USA]) and sequenced using a liquid chromatography electrospray ionization tandem mass spectrometry system (ProteomeX System with LTQ-Orbitrap mass spectrometer, Thermo-Fisher Scientific) in a nanospray configuration. The acquired spectra were searched against the NCBI.fasta protein database narrowed to a subset of human proteins using the SEQUEST search engine (BioWorks 3.1SR software from Thermo-Fisher Scientific). The TurboSEQUEST® search parameters were set as follows: Threshold Dta generation at 10000, Precursor Mass Tolerance for the Dta Generation at 1.4, Dta Search, Peptide Tolerance at 1.5 and Fragment Ions Tolerance at 1.00. Charge state was set on “auto”. Database nr.fasta was retrieved from ftp.ncbi.nih.gov and used to create ‘inhouse’ an indexed human.fasta.idx (keywords: Homo sapiens, human, primate). Proteins with two or more unique peptide sequences (p<0.05) were considered highly confident.

siRNA Treatment & Western Blotting

siRNA was combined with magnetic beads, and MDMs were transfected as indicated by the manufacture's instructions and then cultured for an additional 72 hours in order to achieve maximal protein knockdown. Protein removal was confirmed by Western blotting. Protein samples were quantified using the Pierce 660-nm Protein Assay and Pre-diluted Protein Assay BSA Standards to standardize the curve (Thermo Scientific [IL, USA]). From each protein sample, 10-15 μg was loaded and electrophoresed on a NuPAGE® Novex 4-12% Bis-Tris gel (Invitrogen); the gel was transferred to a polyvinylidene fluoride membrane (Bio-Rad Laboratories [CA, USA]). The membrane was blocked with 5% powdered milk/5% BSA in PBS-T and then probed with primary Ab followed by secondary Ab. Protein bands were distinguished using SuperSignal® West Pico Chemiluminescent substrate (Pierce [IL, USA]). siRNA-transfected MDMs were then treated with 100 μM RTV-NPs followed by harvesting of cells and replicate media samples and drug analysis by HPLC.

### Macrophage RTV-NP Release & Dissociation to Free Drug

Cell culture medium was collected from RTVNP-loaded MDMs 18 hours after drug loading to cells. Intact RTV-NPs were separated from dissolved drug by centrifugation at 100,000×g on a Beckman TL-100 Ultracentrifuge (Brea [CA, USA]) for 1 hour at 4° C. The resulting supernatants and NP pellets were processed for drug quantitation by HPLC.

### Antiretroviral Activities of RTV

Monocyte-derived macrophages were treated with equal amounts of RTV either in the non-formulated state dissolved in ethanol (0.01% final concentration), native RTVNPs or released RTV-NPs for 12 hours and then washed. Drug-treated MDMs were infected with HIVADA at a multiplicity of infection of 0.01 infectious viral particles/cell (Gendelman et al. (1988) J. Exp. Med., 167:1428-1441) on day 1 after RTV-NP treatment. Following viral infection, cells were cultured for 10 days with half media exchanges every other day. Media samples were collected 10 days after infection for measurement of progeny virion production as assayed by reverse transcriptase (RT) activity (Kalter et al. (1991) J. Immunol., 146:3396-3404). Parallel analyses for expression of HIV p24 antigen in infected cells were performed by immunostaining using p24 mouse monoclonal Ab (Dako [CA, USA]) on the same day as media sampling.

### RT Assay

In a 96-well plate, media samples (10 μl) were mixed with 10 μl of a solution containing 100 mM Tris-HCl (pH 7.9), 300 mM KCl, 10 mM dithiothreitol, 0.1% nonyl phenoxylpolyethoxylethanol-40 (NP-40) and water. The reaction mixture was incubated at 37° C. for 15 minutes and 25 μl of a solution containing 50 mM Tris-HCl (pH 7.9), 150 mM KCl, 5 mM dithiothreitol, 15 mM MgCl₂, 0.05% NP-40, 10 μg/ml poly(A), 0.250 U/ml oligo d(T) 12-18 and 10 μCi/ml tritiated thymidine triphosphate was added to each well; plates were incubated at 37° C. for 18 hours. Following incubation, 50 μl of cold 10% TCA was added to each well; the wells were harvested onto glass fiber filters, and the filters were assessed for tritiated thymidine triphosphate incorporation by β-scintillation spectroscopy using a TopCount NX™ (PerkinElmer Inc. [MA, USA]) (Kalter et al. (1991) J. Immunol., 146:3396-3404).

### Immunohistochemistry & Quantitation of HIV-1 p24 Staining

A total of 10 days after HIV-1 infection, cells were fixed with 4% phosphate-buffered PFA for 15 minutes at room temperature. Fixed cells were blocked with 10% BSA in PBS containing 1% Triton X-100 for 30 minutes at room temperature and incubated with mouse monoclonal antibodies to HIV-1 p24 (1:100, Dako) for 3 hours at room temperature. Binding of p24 Ab was detected using a Dako EnVision™+System-HRP labeled polymer anti-mouse secondary Ab and diaminobenzidine staining. Cell nuclei were counterstained with hematoxylin for 60 seconds. Images were taken using a Nikon TE300 microscope with a 40× objective. Quantitation of immunostaining was performed by densitometry using Image-Pro Plus, v. 4.0 (Media Cybernetics Inc. [MD, USA]). Expression of p24 was quantified by determining the positive area (index) as a percentage of the total image area per microscopy field.

### Statistical Analyses

Quantitation of immunostaining was performed with ImageJ software, utilizing JACoP plugins (rsb.info.nih.gov/ij/plugins/track/jacop.html) to calculate Pearson's colocalization coefficients. Comparison was performed on three to five sets of images acquired with the same optical settings. Graphs and statistical analyses were generated using Excel and Prism software (GraphPad Software, Inc. [CA, USA]). Significant differences in drug levels in uptake and release studies were determined by two-way ANOVA followed by Bonferroni's Multiple Comparison Test. Significant differences in RT activity, p24 density and drug content in siRNA experiments were determined by one-way ANOVA followed by Dunnett's Multiple Comparison Test. Two-tailed Student's t-tests were used for all other data; and unless otherwise noted, the error bars are shown as ±standard error of the mean (SEM). Results were considered significant at p<0.05.

### Results

### Characterization & In Vitro Pharmacokinetics of RTV-NPs

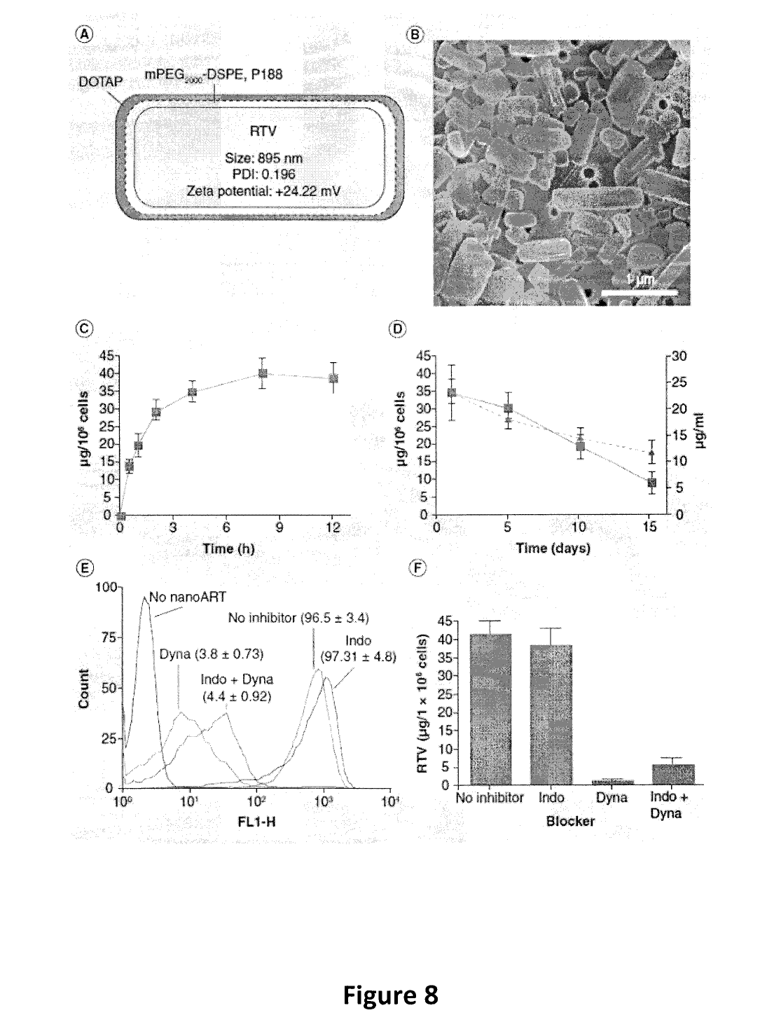

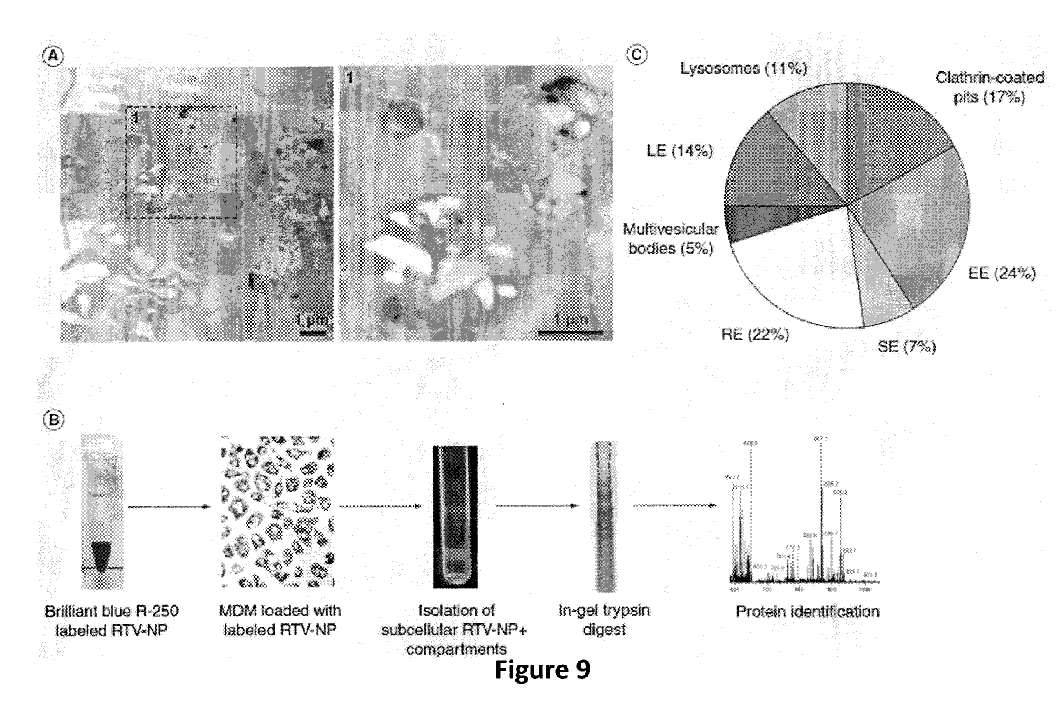

Ritonavir NPs were a representative formulation of nanoART and used as such for assays of cell particle localization and release. The RTV-NP consisted of drug crystals of free-base RTV coated with a thin layer of phospholipid surfactants of mPEG₂₀₀₀-DSPE, P188 and DOTAP. Physical properties (size, shape and zeta potential) of the particles are shown in FIG. 8A. P188 and mPEG₂₀₀₀-DSPE increased particle stability, while the DOTAP coating enabled a positive surface charge. The polydispersity index was 0.196, indicating that while the majority of RTV-NPs were the calculated average measured size, the overall particle population was heterogeneous. Of note, P188 alone, P188/mPEG₂₀₀₀-DSPE or P188/mPEG₂₀₀₀-DSPE-DOTAP do not affect RTV-NP cell uptake. Scanning electron microscopy revealed smooth rod-like morphologies for the RTV-NPs and confirmed size measurements and distribution (FIG. 8B).

The cell-based pharmacokinetics of RTV-NP MDM uptake and release were assessed. Cells were exposed to 100 μM RTV-NPs in DMEM and drug uptake was assessed by HPLC. This drug concentration was chosen based upon previous observations that demonstrated it had limited cellular toxicity and potent antiretroviral efficacy when assayed by (3-(4,5-dimethylthiazol-2-yl)-2,5-diphenyltetrazolium bromide, a yellow tetrazole) assay and RT activity, respectively. RTV-NP internalization in MDMs was observed at 30 minutes and peaked at 4 hours (FIG. 8C). Internalized particles were detected in MDMs for up to 15 days (FIG. 8D). To assay the mechanism of RTV-NP entry into macrophages, chemical blockers were used to inhibit clathrin- and caveolae-mediated cell entry. After pretreatment with 100 μM dynasore (prevents clathrin-coated pit formation through inhibition of dynamin) or indomethacin (caveolae inhibitor) for 1 hour, MDMs were exposed to fluorescent RTV-NPs. Flow cytometry and HPLC analyses demonstrated that RTV-NP internalization occurs through clathrin-coated pits (FIGS. 8E & 8F).

### Proteomic Analysis Identifies RTV-NP Containing Endocytic Compartments

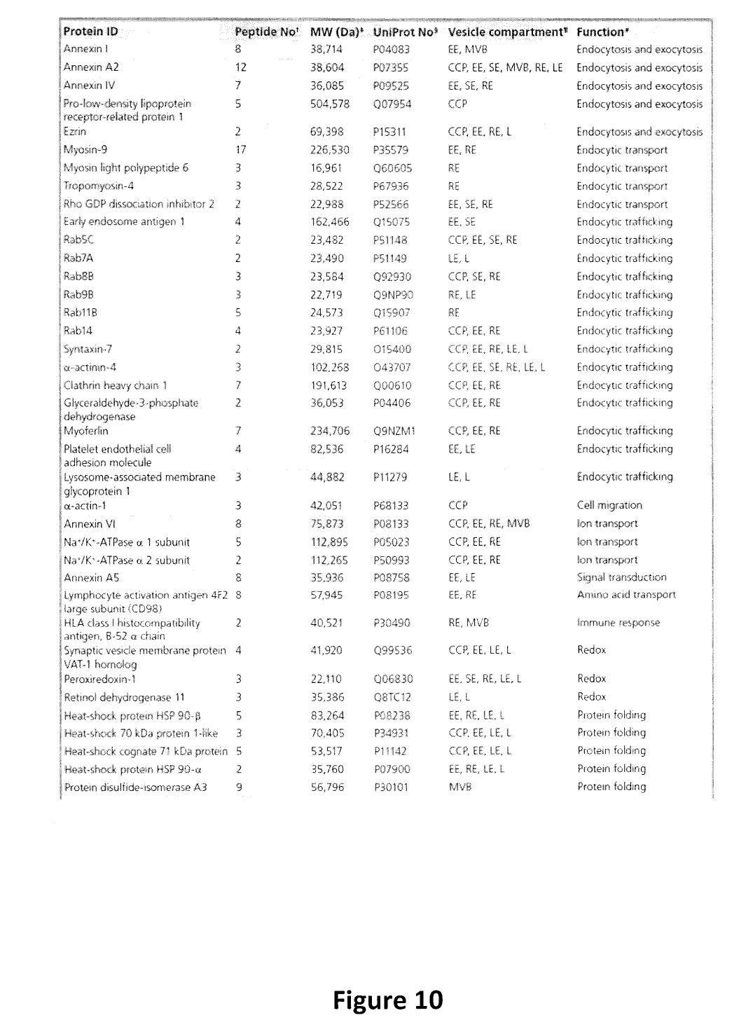

Imaging of RTV-NP-laden MDMs by transmission electron microscopy confirmed uptake of intact particles into distinct cytoplasmic vesicles (FIG. 9A). The subcellular distribution of RTVNPs within MDMs was then investigated. RTV-NPs were labeled with Brilliant Blue R-250 dye and added to MDMs for 12 hours. MDMs were mechanically disrupted and RTV-NPs containing endocytic compartments (blue) were collected as blue bands on a sucrose gradient (FIG. 9B). Mass spectrometry analyses of the fractions identified 38 proteins associated with distinct endosomal populations (FIG. 10). Based on their postulated subcellular localization, the proteins were classified into clathrin-coated pits, early endosomes (EE), recycling endosomes (RE), multivesicular bodies, late endosomes (LE), and lysosomes (L). Proteins were distributed predominantly in early and recycling endocytic cell compartments (EE [24%] and RE [22%]) (FIG. 9C). These data indicate the role of recycling compartments in intracellular sorting of RTV-NPs.

### Confocal Microscopy for RTV-NP Subcellular Distribution

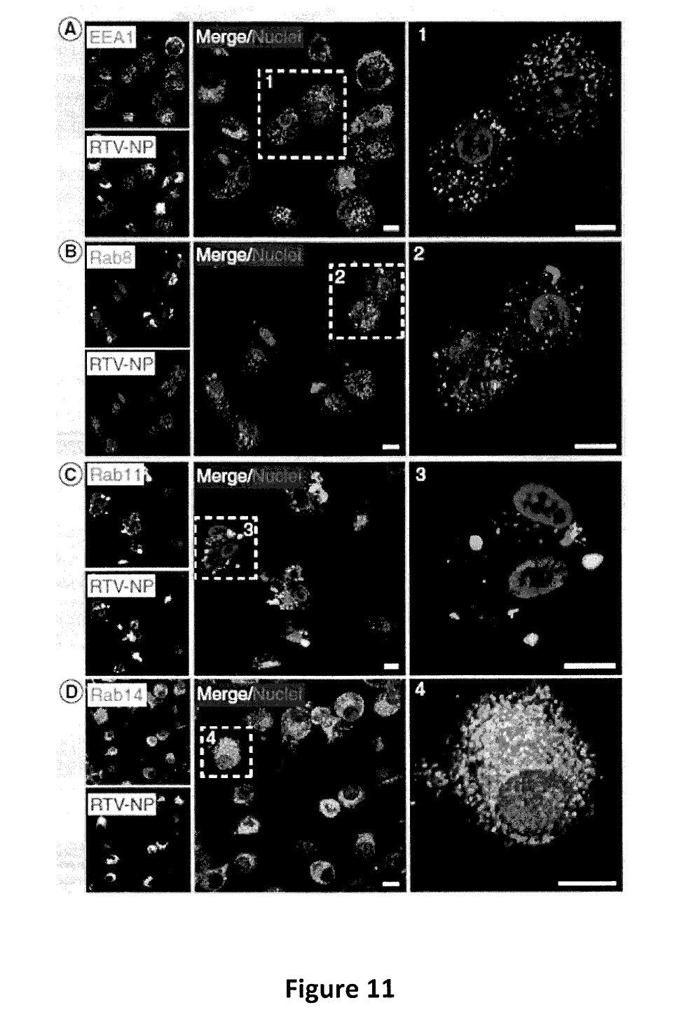

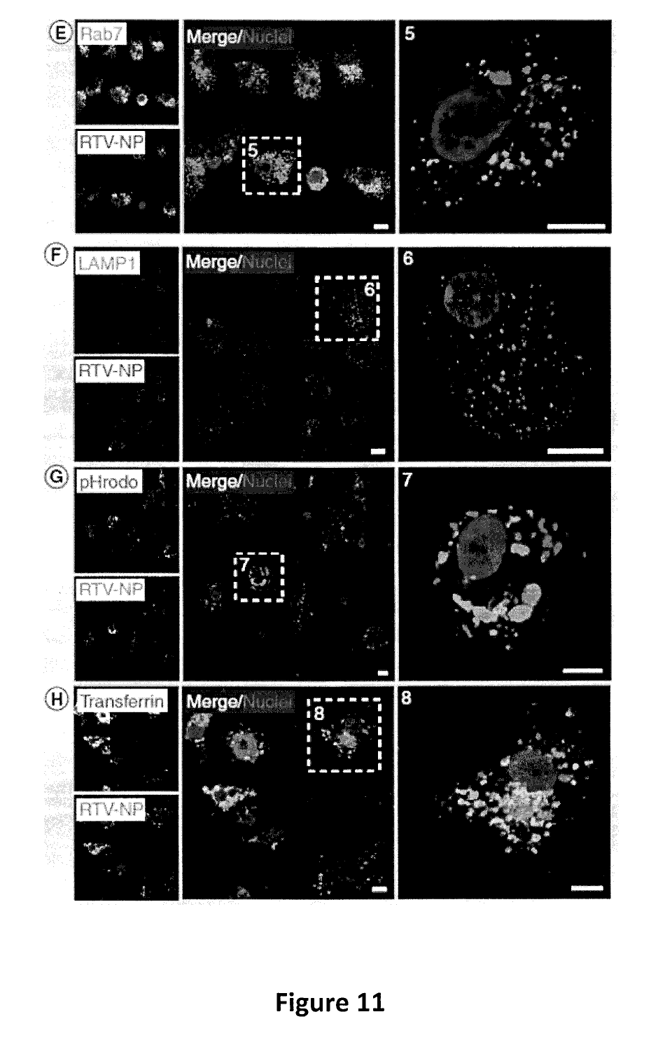

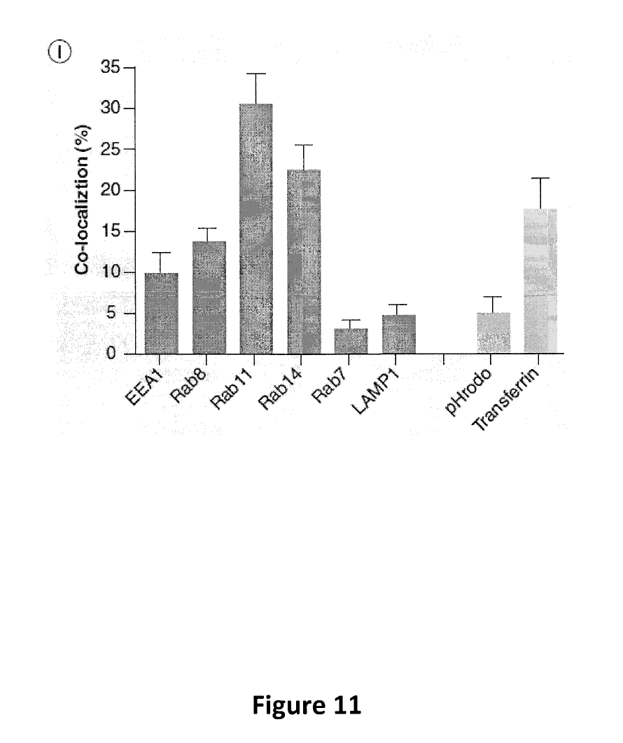

To further substantiate proteomic findings, confocal microscopy was performed to visualize and quantitate the subcellular distribution of RTV-NPs with early (EEA1), recycling (Rab8, 11, 14) and degrading endocytic compartments (late degrading endosomes [Rab7]) and L (LAMP1). MDMs were treated with RTVNPs fluorescently labeled with either DiD or DiO for 12 hours and then immunostained for the endocytic compartments identified by proteomic analysis (FIG. 11). Confocal imaging showed NP distribution in a punctate pattern throughout the cytoplasm and perinuclear region colocalizing predominantly with EE and RE (FIG. 11A-H). Quantitation of fluorophore overlap using Pearson's co-localization tests showed significant accumulation (p<0.001) of RTV-NPs with EEA1+ (9.5±0.4% [mean±SEM]; n=41) early endosomes and Rab8+ (12.8±0.2%; n=56), Rab11+(31.0±0.6%; n=33) and Rab14+ (22.8±0.5%; n=189) RE compared with degrading compartments such as Rab7 LE (4.1±0.4%; n=47) and L (5.0±0.9%; n=28; FIG. 11G). These data indicate that the particles undergo endocytic recycling rather than degradation in human MDMs.

Disruption of Endocytic Recycling with siRNA & Brefeldin A Blocks RTV-NP Release

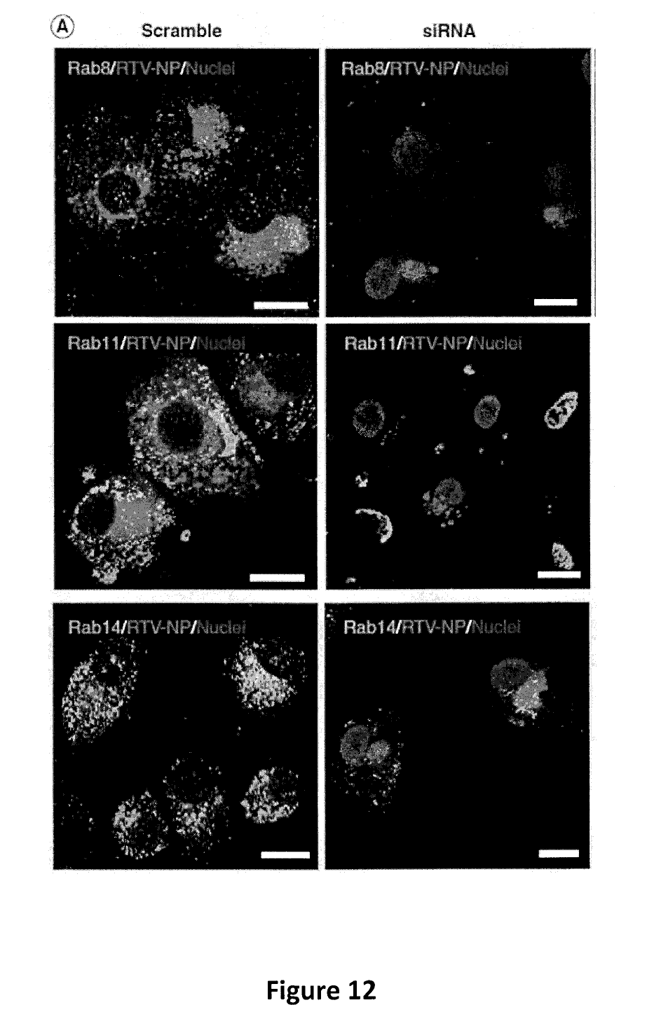

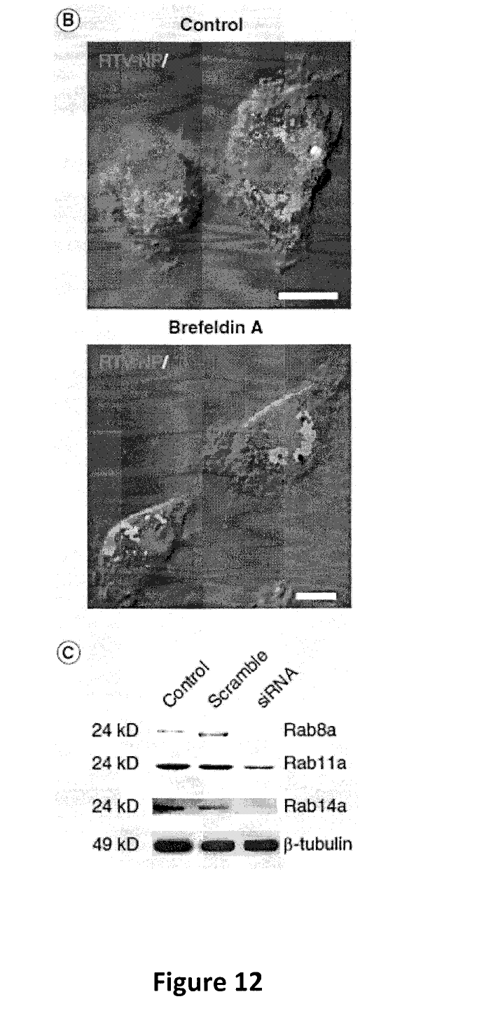

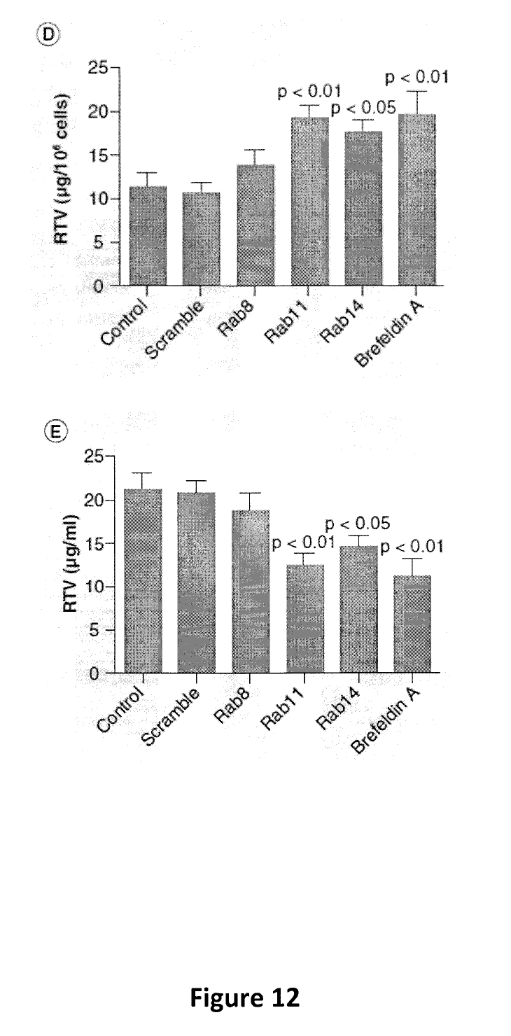

To confirm the recycling pathway for RTV-NP trafficking, Rab8, 11 and 14 were suppressed by siRNA. MDMs exposed to siRNA and DiD-labeled RTV-NPs were analyzed by confocal microscopy for cellular distribution and HPLC for drug retention (cell lysates) and release (culture medium). Western blots using untreated MDMs and those exposed to either scrambled or targeted siRNA confirmed Rab protein silencing (FIG. 12C). Confocal microscopy revealed that in siRNA-treated MDMs the distribution of RTV-NPs was considerably altered compared with untreated and scramble-treated MDMs (FIGS. 11 & 12A). Treated cells displayed a diminished codistribution of Rab8, 11 and 14 with RTV-NPs, loss of cytoplasmic punctate distribution and aggregation of the particles adjacent to the nucleus (FIG. 12A). HPLC analyses indicated that cells treated with Rab11 and Rab14 siRNA retained more drug (FIG. 12D) and released less drug (FIG. 12E) into the media than untreated, siRNA scramble- and Rab8 siRNA-treated MDMs. Treatment with brefeldin A (BFA; a disruptor of recycling and secretory activities) yielded similar results, resulting in aggregation of RTV-NPs in the perinuclear region (FIG. 12B). HPLC analyses showed that MDMs treated with BFA retained more drug and released less drug than untreated and siRNA scramble-treated cells (FIGS. 12D & 12E). Consequently, considerably fewer RTV-NPs were detected in the culture media of the BFA-treated cells. Together these data demonstrate endocytic recycling routes in both intracellular trafficking and release of RTV-NPs.

### Intact RTV-NP Traffic Between Endocytic Compartments

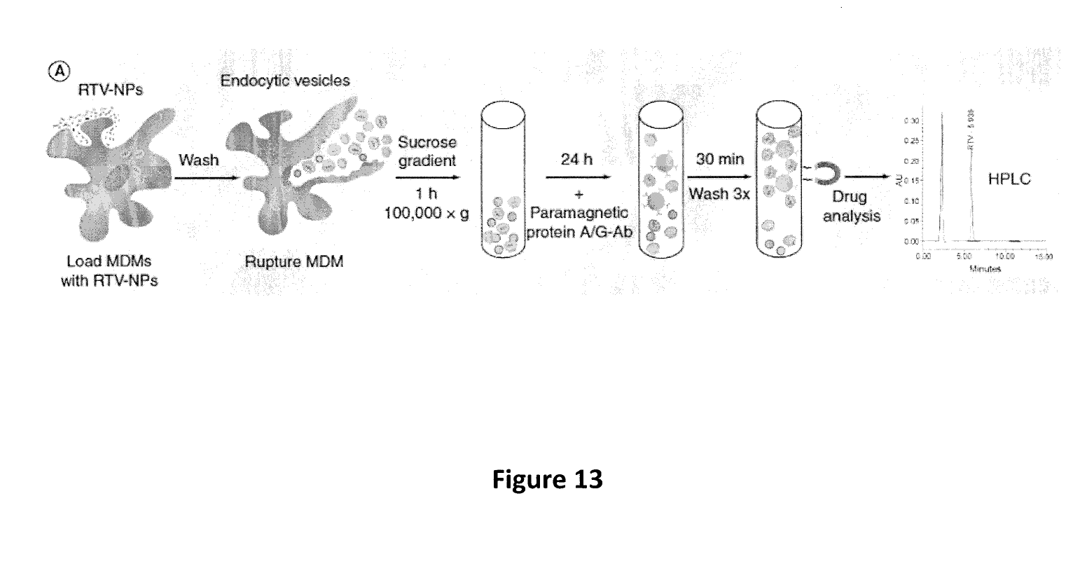

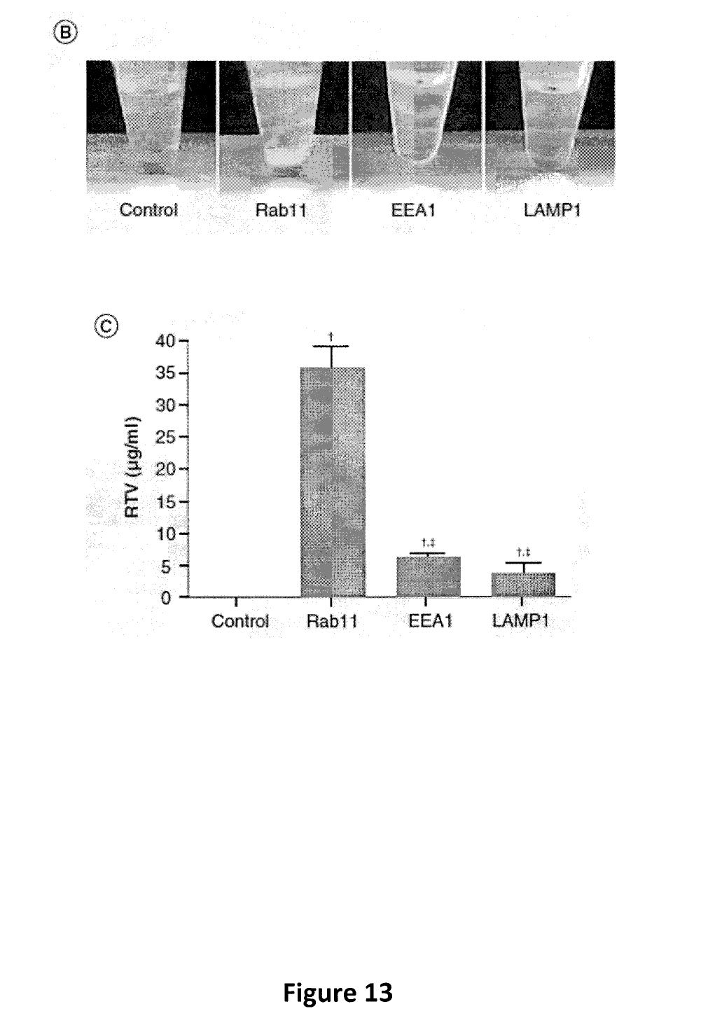

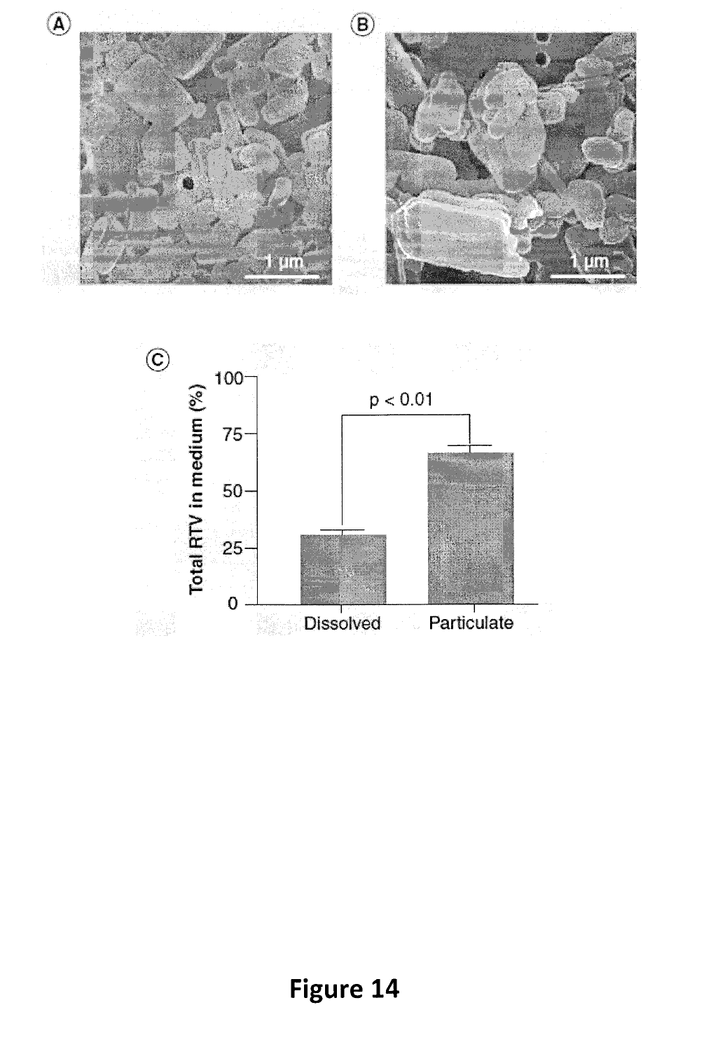

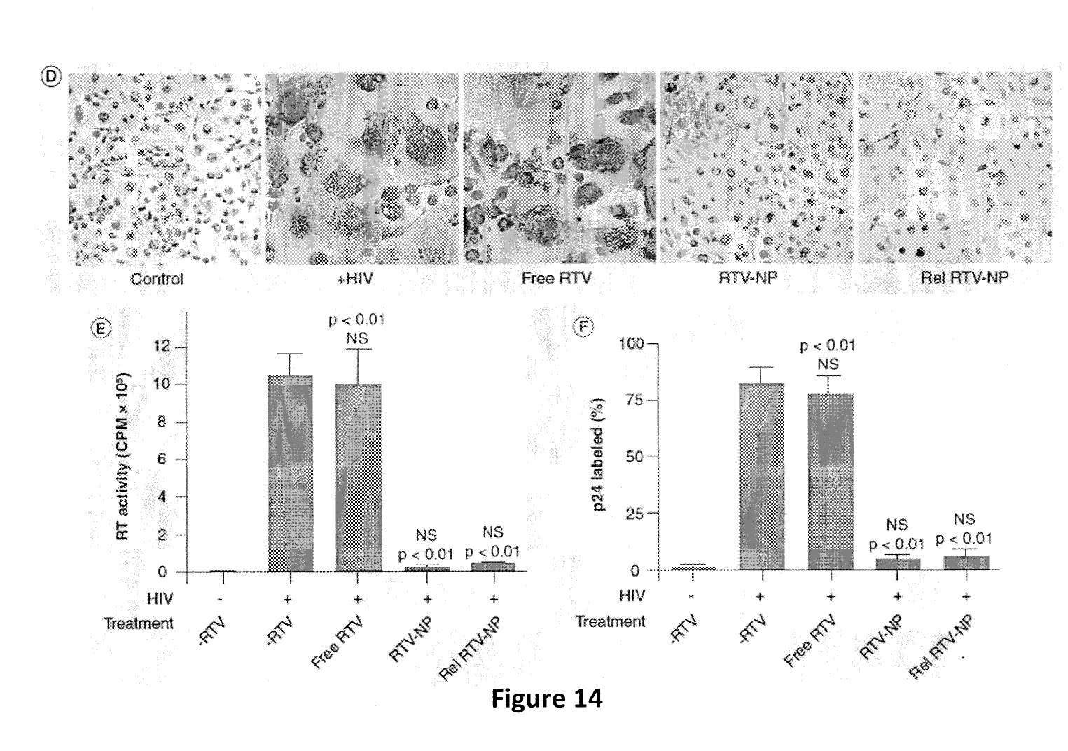

Minimal distribution of RTV-NPs with late degrading endosomes and L led us to question whether indeed the drug bypassed degradation pathways. Since only the surfactants (not the drug crystals) were fluorescently labeled with the lipophillic dyes DiD and DiO, it was investigated whether the particle was trafficked intact. Cells exposed to RTV-NPs were mechanically disrupted and the EE, RE and L were immune-isolated using protein A/G paramagnetic beads conjugated to EEA1, Rab11 and LAMP1 (FIG. 13A). Endocytic compartments bound to beads were collected by magnetic separation, digitally imaged and then analyzed by HPLC for drug content (FIG. 13A). Interestingly, a thin layer of drug (white) covering the bead pellet (brown) was readily visible in the immunoisolated Rab11 endosomes but not in other compartments (FIG. 13B). Consistent with co-localization confocal tests, HPLC analysis confirmed the presence of drugs in Rab11 endosomes and, more importantly, at significantly greater amounts compared with EEA1- or LAMP1-associated vesicles (FIG. 13C). These results indicate that the polymer coat and the drug crystal parts of RTV-NPs indeed undergo the same sorting (recycling) routes. To determine whether the RTV-NPs are preserved during endocytic trafficking and released intact, RTV-NPs collected from culture fluids 24 hours post-uptake were imaged in addition to measuring drug content. To avoid collection of original RTV-NPs, MDMs were exposed to DiD-labeled RTV-NPs for 12 hours, thoroughly washed (five times in 1 ml of PBS), imaged with fluorescent microscopy to confirm the presence of only intracellular particles, and then allowed to release drug for 24 hours post-uptake. Scanning electron microscopy images show that released RTV-NPs are intact (FIG. 14B) and display the same size and shape as the original particles (FIG. 14A). However, the released particles displayed ragged edges and were released as aggregates (FIG. 14B) as opposed to native particles that had smooth edges and were all individually distinct (FIG. 14A). Since released RTV-NPs were identified in cell culture fluids, it was determined that the relative amount of drug that was present in particulate form compared with dissolved free drug. Particulate RTV was separated from soluble RTV by ultracentrifugation and quantitated by HPLC. As shown in FIG. 14C, of the total amount of RTV, 32% was dissolved in culture medium, while 68% was present as intact NPs. These data indicate that the majority of drug is released from cells in particulate form.

Minimal distribution with acidic (degrading) compartments, as labeled by dextran-conjugated pH-sensitive dye (fluoresces bright red at pH<5.5), suggests that the mild pH environment of RE may preserve the integrity of RTV-NPs (FIG. 11G). Additionally, considerable overlap of RTV-NPs with Tfn+ compartments indicates that fast recycling to the plasma membrane may also contribute to the preservation of intact particles (FIG. 11E). These results indicate that RTV-NP physical properties and morphology are not affected during trafficking within the macrophage.

RTV-NPs Maintain Antiretroviral Activities after Cell Release

To test whether antiretroviral activities were maintained after particle release, MDMs were exposed to equal concentrations of native RTV-NPs, released RTV-NPs and free drug followed by a challenge with HIV infection. Antiretroviral effects were measured by HIV p24 expression and RT activity. Pretreatment with free RTV provided no protection against infection, while both native RTV-NPs and released RTV-NPs were equally able to significantly (p<0.01) suppress HIV replication (p24 staining) and formation of multinucleated giant cells (FIGS. 14D & 14F). Reverse transcriptase activity was significantly suppressed in MDMs exposed to original and released RTV-NPs compared with untreated cells and those exposed to free drug (FIG. 14E). These findings demonstrate that endocytic sorting does not affect antiretroviral efficacy of RTV-NPs and suggests a high potential of the macrophage as an efficient drug-laden particle delivery vehicle.

Reformulating ART drugs into nanocrystals for transport by mononuclear phagocytes (MPs; monocytes and tissue macrophages) improves clinical drug efficacy. Indeed, harnessing MPs as vehicles for drug delivery to HIV sanctuaries protected by biological barriers serves both to simplify drug regimens and enhance their therapeutic index. ART medications are insoluble in water and thus can form stable crystals in aqueous solutions. Owing to their phagocytic and migratory functions MPs can readily ingest foreign material and cross into areas of microbial infection and inflammation. If loaded with drug NPs, these cells deliver drug(s) to sites that would otherwise be inaccessible due to the presence of either physical or biochemical barriers. MPs are ideal candidates for transporting nanoART since the cells are HIV targets and can act both as viral reservoirs and transporters. Notably, nanoformulations have been developed for cancer chemotherapy and for a range of microbial infections (e.g., Blyth et al. (2010) Cochrane Database Syst. Rev. 2:CD006343; Chu et al. (2009) Curr. Med. Res. Opin., 25:3011-3020; Pagano et al. (2010) Blood Rev. 24:51-61; Destache et al. (2009) BMC Infect. Dis., 9:198; Destache et al. (2010) J. Antimicrob. Chemother., 65:2183-2187; Beduneau et al. (2009) PLoS ONE 4:e4343; Brynskikh et al. (2010) Nanomed., 5:379-396; Gorantla et al. (2006) J. Leukoc. Biol., 80:1165-1174; Liu et al. (2008) J. Neuroimmunol., 200:41-52). Further, the notion that MP migratory function can be harnessed for therapeutic benefit makes practical sense as these same cells are viral targets and carriers, show robust phagocytic capabilities and readily migrate to areas of sustained viral growth and inflammation. Notably, the interactions between nanoART and macrophages is important if therapeutic translation is to be achieved. Interestingly, the majority of RTV-NPs were contained within compartments that provide a protected environment and allows for the particles to be released intact with retained antiretroviral activities. Importantly, RTV-NP endocytic compartments mirror those used in the HIV lifecycle. These results strongly indicate that cell-mediated delivery of active drug is effective.

Based on the limited cytotoxicities, sustained high levels of antiretroviral drug levels in virus-targeted tissues (including the lymphoid system and CNS) can be realized as observed in adoptive cell transfers. For widespread use in the clinic, nanoART synthesis will have to be scaled. Techniques such as precipitation, homogenization and wet milling may be used to prepare nanoART with adequate physical and chemical stability for sufficient drug loading, appropriate osmolarity, viscosity and sterility (Marre et al. (2010) Chem. Soc. Rev., 39:1183-1202; Muchow et al. (2008) Drug Dev. Ind. Pharm., 34:1394-1405; Takatsuka et al. (2009) Chem. Pharm. Bull. (Tokyo) 57:1061-1067).

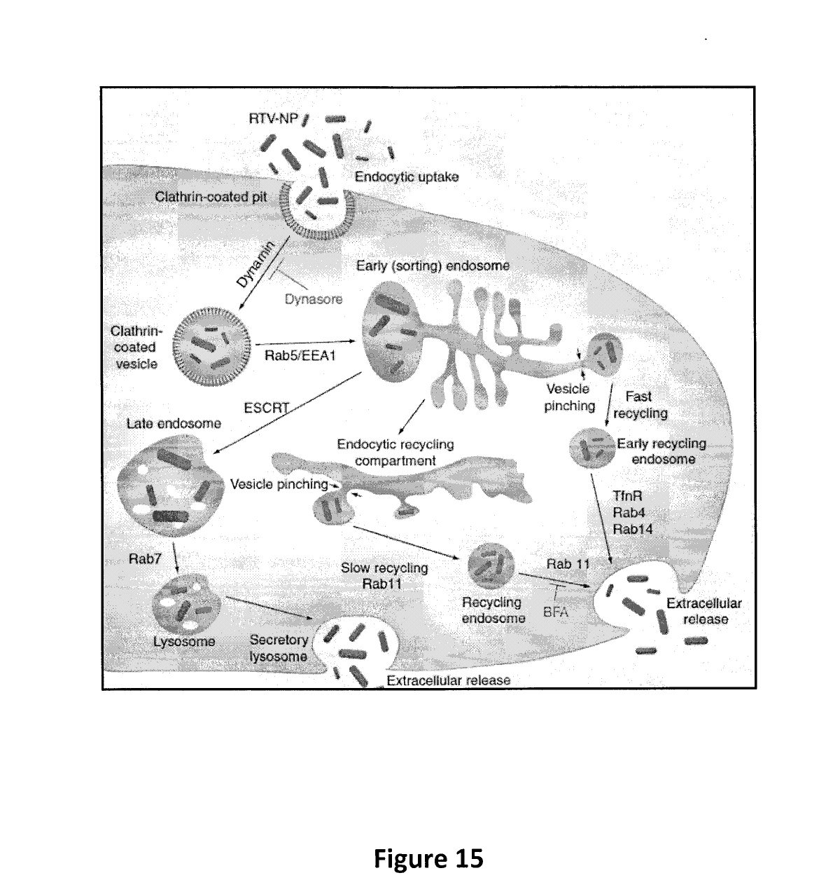

Herein, it is demonstrated that the NPs primarily enter macrophages through a clathrin-mediated pathway (Kumari et al. (2010) Cell Res. 20:256-275). The subcellular distribution of the NPs were seen in recycling endosomal compartments. Indeed, co-localization immunocytochemical studies demonstrated that there were significantly more RTV-NPs in RE, particularly within Rab11+ vesicles, than in other compartments. The subcellular distribution pattern of RTV-NPs was concentrated in the perinuclear region, further supporting their localization to RE (Hattula et al. (2006) J. Cell Sci., 119:4866-4877; Junutula et al. (2004) Mol. Biol. Cell, 15:2218-2229; Lombardi et al. (1993) EMBO J. 12:677-682; Seaman, M. N. (2008) Cell Mol. Life. Sci., 65:2842-2858). Although RTV-NP presence was also observed in LE and L due to limited particle overlap with acidic vesicles, it is likely that the particles contained within LE and L are not being degraded but redirected to recycling compartments. For example, Rab9, which was identified by proteomic analysis, is involved with the retrograde transport of LE that eventually fuse with the trans-Golgi network and are packaged into RE (Barbero et al. (2002) J. Cell Biol., 156:511-518; Bonifacino et al. (2006) Nat. Rev. Mol. Cell. Biol., 7:568-579). These results suggested that RTV-NPs avoided intracellular degradation and were primarily being stored within recycling compartments for eventual release at the cell surface. Functional studies demonstrated that the removal of proteins involved with the trafficking of RE inhibited drug release from RTV-NP-containing cells. In particular, Rab11, a marker for intracellular recycling (Hoekstra et al. (2004) J. Cell Sci., 117:2183-2192; Hsu et al. (2010) Curr. Opin. Cell Biol., 22:528-534; Jing et al. (2009) Histol. Histopathol. 24:1171-1180; Jones et al. (2006) Curr. Opin. Cell Biol. 18:549-557; Maxfield et al. (2004) Nat. Rev. Mol. Cell Biol. 5:121-132; Sonnichsen et al. (2000) J. Cell Biol. 149:901-914; Zerial et al. (2001) Nat. Rev. Mol. Cell Biol. 2:107-117), appeared to facilitate both particle trafficking and release. There are two main types of endosomal recycling: slow and fast. Rab11 along with Rab9, both of which were identified during proteomic analyses, have been recognized as proteins that participate in slow recycling (Cayouette et al. (2010) Biochim. Biophys. Acta 1803:805-812; Sheff et al. (1999) J. Cell Biol. 145:123-139; Ullrich et al. (1996) J. Cell Biol. 135:913-924; van der Sluijs et al. (1992) EMBO J. 11:4379-4389). In addition, Rab11 has been shown to play a role in exocytosis in that it can control the passage of material from the Golgi through endosomes and finally to the cell surface, known as slow recycling, as opposed to Rab8 and 14, which direct transit from the Golgi directly to the cell surface, known as fast recycling (Chen et al. (2001) Methods Enzymol. 329:165-175; Larance et al. (2005) J. Biol. Chem. 280:37803-37813). This could explain the differences seen in the functional consequences of removal of the Rab proteins. In all cases, removal of the recycling Rab proteins caused the RTV-NPs to redistribute very densely near the nucleus, suggesting that all of these proteins are involved with particle trafficking. However, removal of Rab8 did not inhibit drug release and removal of Rab14 only slightly reduced it. On the other hand, removal of Rab11 had a very significant inhibitory effect, suggesting that while some particles may be released quickly, the primary mechanism probably involves slow recycling to the plasma membrane via Rab11+ vesicles. It is worth noting that Rab11 has been implicated in the rapid recycling of Tfn (Cox et al. (2000) Proc. Natl. Acad. Sci. 97:680-685), and an appreciable amount of particle co-localization with Tfn was observed. It is probable that rapid recycling of RTV-NPs does occur since removal of Rab11 did not totally inhibit drug release; however, since RTV-NPs persist in MDMs for over 2 weeks, it is more likely that the majority of particles are indeed slowly recycled. Without being bound by theory, a proposed pathway for intracellular trafficking of RTV-NPs from uptake to release is shown in FIG. 15. Finally, Rab11 is known to recruit both actin- and microtubule-based motor protein complexes that transport vesicles along cytoskeletal filaments (Jordens et al. (2005) Traffic 6:1070-1077). Many of these proteins were also identified during the proteomic analyses.

Taken together, these data uncover a pathway in which RTV-NPs avoid intracellular degradation and are recycled to the plasma membrane. This was demonstrated by visually identifying intact RTV-NPs that had been released from particle-laden MDMs. It was further demonstrated that these released particles retained full antiretroviral activity. In this regard, MDMs uptake, retain, transport and release intact RTV-NPs that inhibit HIV replication, indicating that macrophages can act as true ‘Trojan horses’ for nanoART, delivering active drug(s) to sites of viral infection. Second, it appears that RTV-NPs can inhibit viral replication via an intracellular mechanism since a small amount of RTV-NPs was able to completely suppress viral replication, while an equivalent amount of free drug had no effect. This facet of NP-macrophage interactions supports the idea that RTV-NPs, like HIV, enter macrophages through clathrin-coated pits (Vendeville et al. (2004) Mol. Biol. Cell 15:2347-2360). In addition, a significant component of the virus' lifecycle occurs within RE (Murray et al. (2005) J. Virol. 79:11742-11751; Varthakavi et al. (2006) Traffic 7:298-307). Together, these findings indicate that RTV-NPs could not only enter the cell along with HIV but also have identical subcellular destinations, thus enabling drug targeting to specific subcellular compartments. This provides a cogent rationale for why nanoART, in the broader sense, could have potent antiretroviral effects even at very low intracellular concentrations. Direct inhibition of HIV replication at the subcellular level would subsequently increase the therapeutic index of ART, thereby decreasing the amount of drug needed to inhibit viral replication.

### Example 3

### Materials and Methods

### Materials and Instruments

Poloxamer 188 (P188; Pluronic® F68), Poloxamer 407 (P407; Pluronic® F-127), and folic acid were obtained from Sigma-Aldrich (Saint Louis, Mo.). N-hydroxysuccinimide, N,N″-dicyclohexylcarbodiimide, and triethylamine were purchased from Acros Organics (Morris Plains, N.J.). LH-20 was obtained from GE HealthCare (Piscataway, N.J.). ATV sulfate was purchased from Gyma Laboratories of America Inc. (Westbury, N.Y.) and then free-based with triethylamine by extraction. All other reagents and solvents if not specified were purchased from Fisher Scientific (Pittsburgh, Pa.) or Acros. ¹H and spectra were recorded on a 500 MHz NMR spectrometer (Varian, Palo Alto, Calif.). Atazanavir nanosuspensions were prepared either by NETZSCH MicroSeries Wet Mill (NETZSCH Premier Technologies, LLC., Exton, Pa.) or by Avestin C5 high-pressure homogenizer.

### Synthesis of Folate Terminated P188 (FA-P188) and P407 (FA-P407)

Synthesis of p-toluenesulfonyl terminated P188 (Tos-P188, 2). P188 (8.4 g, 1 mmol) was dehydrated by coevaporation with toluene (3×50 mL) and then dissolved under argon in 20 mL of anhydrous DCM together with DMAP (61 mg, 0.5 mmol) and TEA (1.01 g, 10 mmol). The reaction mixture was cooled to 0° C. and p-toluenesulfonyl chloride (1.9 g, 10 mmol) was added. After reacted overnight at room temperature, the mixture was filtered, concentrated, and precipitated in ether. The crude product was then extracted with DCM/Brine, the organic layer was dried over anhydrous magnesium sulfide and then concentrated under reduced pressure. The solvent was then precipitated in ether to afford crude product. The analytic pure product was obtained by further purification with LH 20 column. Yield: 6.8 g. ¹H NMR (DMSO-d₆) δ (ppm) 7.78 (d, J=7.32 Hz), 7.48 (d, J=7.32 Hz), 4.11 (t, J=4.39 Hz), 3.64-3.37 (m), 1.04 (d, J=3.90 Hz).

Synthesis of azide terminated P188 (Azido-P188, 3). Tos-P188 (5.22 g, 0.6 mmol) was dissolved in 20 mL DMF, and then sodium azide (0.39 g, 6 mmol) was added. The reaction was carried out with stirring at 100° C. for 2 days. After filtration, the solvent was removed under vacuum. The crude product was dissolved in dichloride methane (20 mL), and extracted with brine (3×15 mL). The organic layer was dried over anhydrous magnesium sulfide. After removal of the organic solvent, the crude product was further purified by LH 20 column to get analytic pure product. Yield: 4.5 g. ¹H NMR (DMSO-d₆) 8 (ppm) 3.65 (t, J=4.39 Hz), 3.61-3.38 (m), 1.04 (d, J=3.90 Hz).

Synthesis of amine terminated P188 (Amine-P188, 4). N3-P188 (1.26 g, 0.15 mmol) and 10 mg of Pd/C (10% wt) were stirred in 10 ml of absolute MeOH at room temperature for 72 hours, in a round bottomed flask, under H₂ atmosphere. The reaction mixture was filtered through filter paper to separate the Pd/C, and the solvent was evaporated. Further purification of the product was carried out by precipitation from dichloromethane/ether mixture at 0° C. The product was filtered and dried in vacuum to give a solid. Yield: 0.9 g. ¹H NMR (DMSO-d₆) δ (ppm) 2.65 (t, J=4.39 Hz), 3.64-3.43 (m), 1.03 (d, J=3.90 Hz).

Synthesis of active ester of folic acid (Folate-NHS, 6). 0.5 g of folic acid is dissolved in 10 ml of DMSO plus 0.25 ml of triethylamine. A 1.1 molar ratio of NHS (0.13 g) and DCC (0.23 g) is added. The mixture is stirred overnight at room temperature in the dark. The by-product, dicyclohexylurea, is removed by filtration. NHS-folate, which is in the filtrate, was precipitated with diethylether and stored as a yellow powder after several washes with anhydrous ether and desiccation. Yield: 0.4 g. ¹H NMR (DMSO-d₆) δ (ppm) 8.66 (s, 1H), 8.16 (d, J=6.8 Hz, 1H), 7.65 (d, J=7.80 Hz, 2H), 6.93 (t, J=5.85 Hz, 1H), 6.64 (d, J=7.80 Hz, 2H), 4.50 (d, J=5.85 Hz, 2H), 4.23 (m, 1H), 2.83 (s, 4H), 2.31 ((t, J=6.83 Hz, 2H)), 2.03 (m, 1H), 1.93 (m, 1H).

Synthesis of folate terminated P188 (FA-P188, 7). Amine-P188 (0.84 g, 0.1 mmol) was dissolved in 10 mL DMSO, Folate-NHS (322 mg, 0.6 mmol) was slowly added into this solution and reacted at room temperature overnight under dark condition. The crude product was precipitated into ether, and further purified with LH 20 column. Yield: 0.6 g. ¹H NMR (DMSO-d₆) δ (ppm) 8.66 (s), 7.65 (d, J=8.78 Hz), 6.92 (t, J=4.88 Hz), 6.64 (d, J=8.78 Hz), 4.48 (d, J=5.37 Hz), 4.28 (m), 3.65 (t, J=4.39 Hz), 3.60-3.41 (m), 2.18 (b), 2.08-1.89 (m), 1.04 (d, J=5.85 Hz).

By using the same procedures described above, more FA-P188 and folate terminated P407 (FA-P407) were synthesized for this study.

### Preparation and Characterization of Folate Atazanavir Nanosuspensions (FA-P188-ATV & FA-P407-ATV)

For the preparation of folate atazanavir nanosuspensions, the following surfactant combinations were used: (1) 0.5% P188 alone; (2) 0.05% FA-P188 and 0.45% P188; (3) 0.1% FA-P188 and 0.4% P188; (4) 0.15% FA-P188 and 0.35% P188; (5) 0.5% P407 alone; (6) 0.025% FA-P407 and 0.475% P407; (7) 0.1% FA-P407 and 0.4% P407; (8) 0.2% FA-P407 and 0.3% P407 were suspended in 10 mM HEPES buffer solution (pH 7.8) separately. Free based ATV (1% by weight) was then added to surfactant solutions. The suspensions were agitated to homogeneous dispersions by using an Ultra-turrax® T-18 rotor-stator mixer. For the preparation of nanosuspensions by wet-milling, the suspension was transferred to a NETZSCH MicroSeries Wet Mill (NETZSCH Premier Technologies, LLC., Exton, Pa.) along with 50 mL of 0.8 mm grinding media (zirconium ceramic beads), and milled from 30 minutes to 1 hour at speeds ranging from 600 to 4320 rpm to prepare ATV nanosuspensions with desired particle size. For the preparation of nanosuspensions by homogenization, the suspension was transferred to an Avestin C5 high-pressure homogenizer and homogenized at 20,000 pounds per square inch for approximately 30 passes or until desired particle size was reached. The particle size, polydispersity, and surface charge were analyzed in a Malvern Nano-Zetasizer (Malvern Instruments Inc., Westborough, Mass.). After the desired particle size was achieved, samples were centrifuged at 10,000×g for 30 minutes at 4° C. The resulting pellet was washed two times with 0.925% sucrose and 0.5% polymer solution, and then resuspended in the respective surfactant solutions along with 0.925% sucrose to adjust tonicity for post homogenization. The ATV concentration in nanosuspensions was determined by using high performance liquid chromatography (HPLC).

### The Uptake, Retention, and Release of FA-ATV Nanosuspensions in MDM

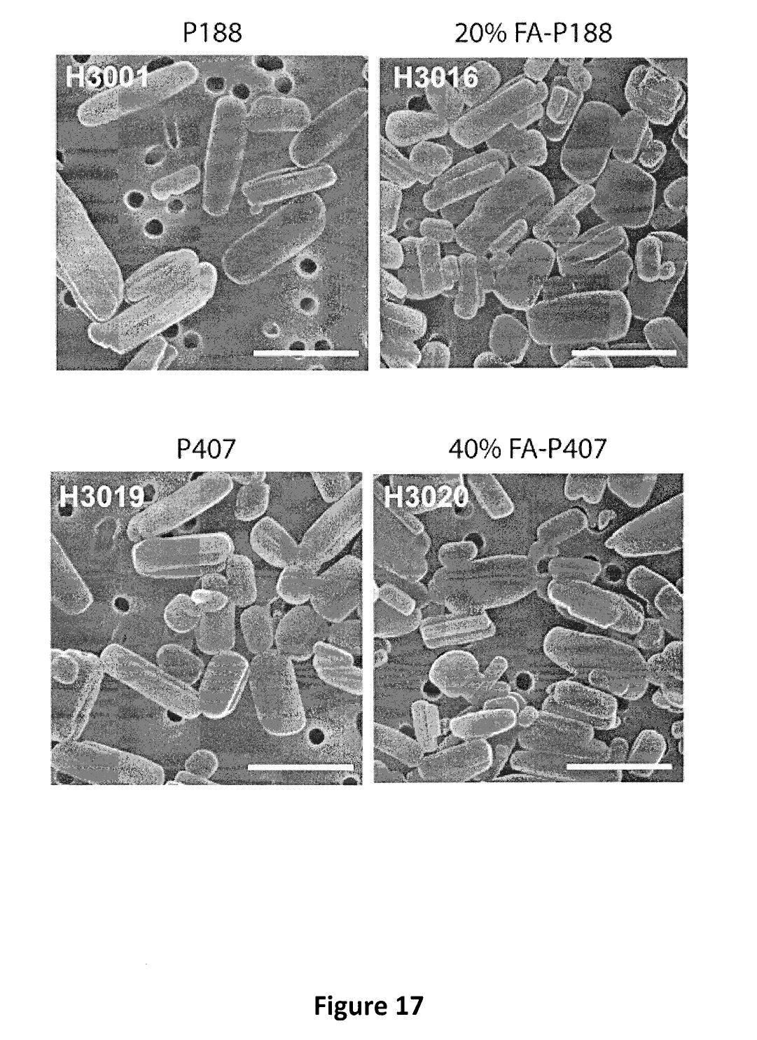

After 7 days of differentiation, monocyte-derived macrophages (MDM) were activated with 0 and 50 ng/mL LPS for 24 hours. Then part of these activated MDM and nonactivated MDM were treated with 100 μM of FA-P188-ATV containing 0%, 10%, and 30% of FA-P188. Another part of these MDM were firstly treated with folic acid and then treated with 100 μM of FA-P188-ATV containing 0%, 10%, and 30% of FA-P188. Uptake of FA-P188-ATV was assessed at different time points without medium change for 8 hours. Adherent MDM were washed with phosphate buffered saline (PBS) and collected by scraping into PBS. Cells were pelleted by centrifugation at 950×g for 10 minutes at 4° C. Cell pellets were briefly sonicated in 200 μl of methanol and centrifuged at 20,000×g for 10 minutes at 4° C. The methanol extract was stored at −80° C. until HPLC analysis (FIG. 17). The uptake of FA-P407-ATV by MDM was evaluated by using the same methods of FA-P188-ATV.

### Antiretroviral Activities of FA-ATV Nanosuspensions

MDM were treated with 100 μM ATV nanosuspensions for 8 hours, washed to remove excess drug, and infected with HIV-1_{ADA} at a multiplicity of infection of 0.01 infectious viral particles/cell on days 10 and 15 post-ATV nanosuspensions treatment. Following viral infection, cells were cultured for ten days with half media exchanges every other day. Medium samples were collected on day 10 for measurement of progeny virion production as assayed by reverse transcriptase (RT) activity. Parallel analyses for expression of HIV-1 p24 antigen by infected cells were performed by immunostaining.

### Results

### Synthesis of Folate-Poloxamers

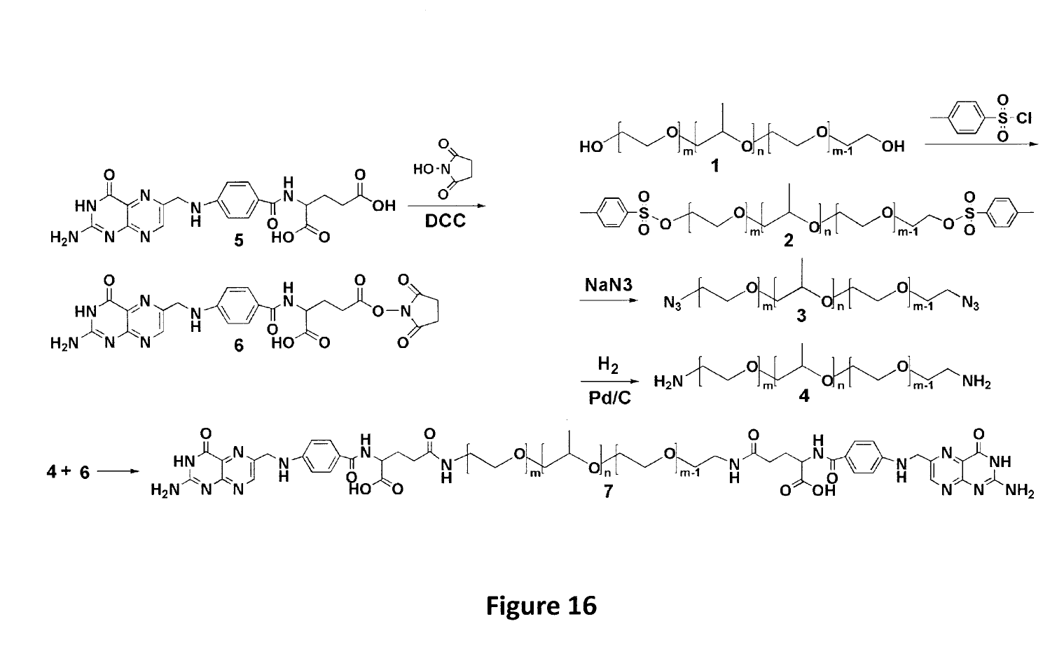

Folate decorated poloxamers were designed and synthesized by the following steps for the targeting delivery of antiretroviral agents to HIV infection sites (FIG. 16). Briefly, after activation of poloxamers (P188 and P407, 1) with excess of p-toluenesulfonyl chloride, the tosylated product (2) was converted to Azido-Poloxamers (3) by reacting with excess of sodium azide in DMF at 100° C. overnight, which was then reduced to Amine-Poloxamers (4) with triphenylphosphine. Finally, folic acid (5) was activated with DCC/NHS, the resulting active ester (6) was reacted with Amine-Poloxamers to get the desired Folate-Poloxamers (FA-P188 and FA-P407, 7). After purification with LH-20 column, pure FA-P188 and FA-P407 (about 2.5 g) were synthesized for MDM uptake, retention, release, and antiretroviral studies.

The nanoformulations used in this study were of similar size, charge and shape. The size of the particles ranged from 281 nm for P188-ATV prepared by homogenization (H3001) to 440 nm for FA-P407-ATV prepared with 5% FA-P407/95% P407 (H3024) as surfactants (Table 3). All particles were negatively charged. The formulation with the highest charge was P188-ATV prepared by homogenization (H3001) and the least negatively charged was the formulation containing 20% FA-P188/80% P188 (H3016). All particles regardless of folate modification or polymer were long rod-shaped (FIG. 17).

TABLE 3
Physicochemical characteristics of nanoformulations of atazanavir.

| | | | | Zeta |
|---|---|---|---|---|
| Formulation | | Size | | Potential^{d} |
| Designation | Surfactant ^{a} | (nm) ^{b} | PDI ^{c} | (mV) |
| H3001 | P188 | 314 | 0.2 | −31.6 |
| M3001 | P188 | 281 | 0.288 | −15.31 |
| H3016 | 20% FA-P188/80% P188 | 385.9 | 0.242 | −4.6 |
| M3016 | 20% FA-P188/80% P188 | 332.1 | 0.195 | −19.6 |
| M3017 | 10% FA-P188/90% P188 | 372.0 | 0.225 | −20.1 |
| M3018 | 30% FA-P188/70% P188 | 379.0 | 0.185 | −19.8 |
| H3019 | P407 | 382.7 | 0.209 | −10.1 |
| H3020 | 40% FA-P407/60% P407 | 364.8 | 0.207 | −24.6 |
| H3022 | 20% FA-P407/80% P407 | 416.8 | 0.318 | −11.5 |
| H3024 | 5% FA-P407/95% P407 | 440.3 | 0.318 | −17.1 |

^{a} Abbreviations used in the table: P188: poloxamer 188; P407: poloxamer 407.
^{b} The particle sizes,
^{c} polydispersity indices (PDI) and
^{d}zeta potential were determined by dynamic light scattering; the z-average diameters are presented.

### The Uptake and Retention of ATV Nanosuspensions by MDM

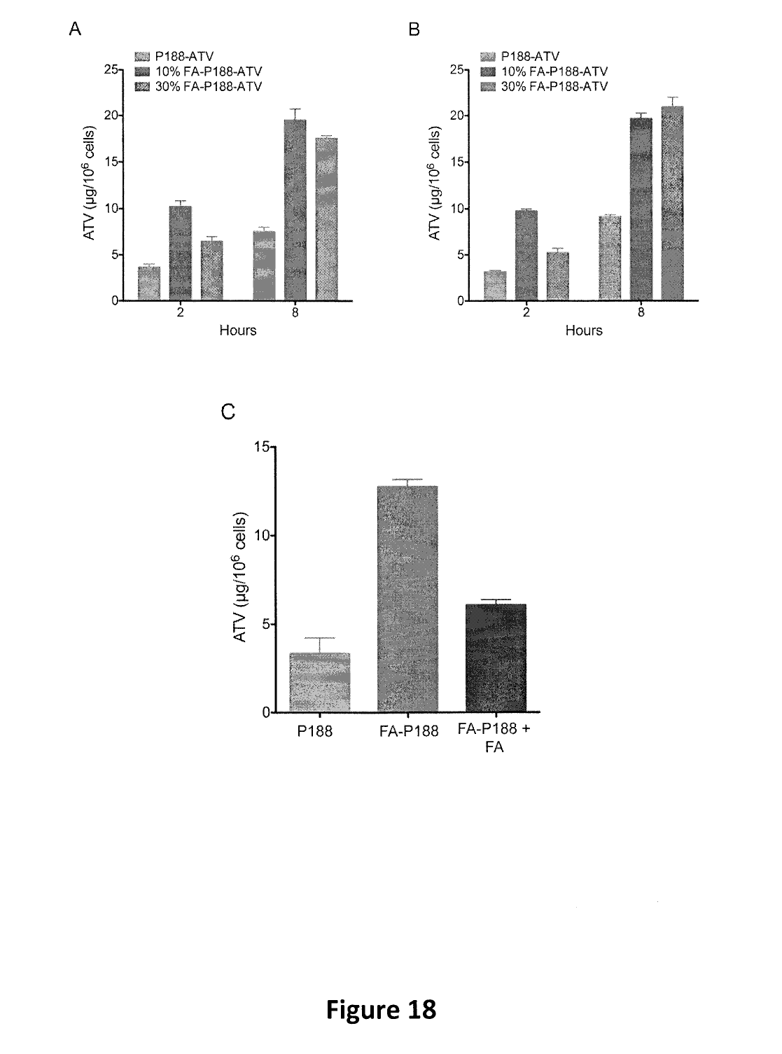

To determine whether folate modification of the polymer coating of the nanocrystals would modify how the nanoformulations were handled by MDM, cellular uptake was determined. The results indicated that expression of the folate receptor is greater on activated macrophages than unactivated macrophages. Thus, it was first determined whether uptake of FA-ATV nanosuspensions would be influenced by activation of cultured MDM. MDM were activated by treatment with 50 ng/ml LPS for 24 hours prior to the addition of ATV nanosuspensions (with or without FA-Poloxamer). Uptake of the nanoformulations was determined at 1 and 8 hours. Eight hours was used for maximum uptake based upon previous studies that demonstrated that >95% of total uptake occurs by 8 hours for most ATV nanosuspensions. Uptake of ATV nanosuspensions decorated with FA-P188 was about 2-fold greater than the uptake of ATV nanosuspensions without FA-P188. Enhanced uptake was not influenced by the percentage of FA-P188 in P188-ATV nanosuspensions (FIG. 18A). Of interest, the enhanced uptake of FA-P188-ATV was not increased by activation of MDM with LPS (FIG. 18B), suggesting that LPS activation does not increase the expression of folate receptors on the cell surface of MDM used. To determine whether the enhanced uptake of FA-P188-ATV was receptor mediated, the folic acid (2.5 mM) was added to the culture medium 30 minutes prior to addition of ATV nanosuspensions. The results showed that addition of excess folic acid blocked the enhanced uptake of folate-modified ATV nanosuspensions, and the uptake ATV nanosuspensions with FA-P188 was similar to that of ATV nanosuspensions without FA-P188 (FIG. 18C), indicating the enhanced uptake and targeting ability of FA-ATV nanosuspensions.

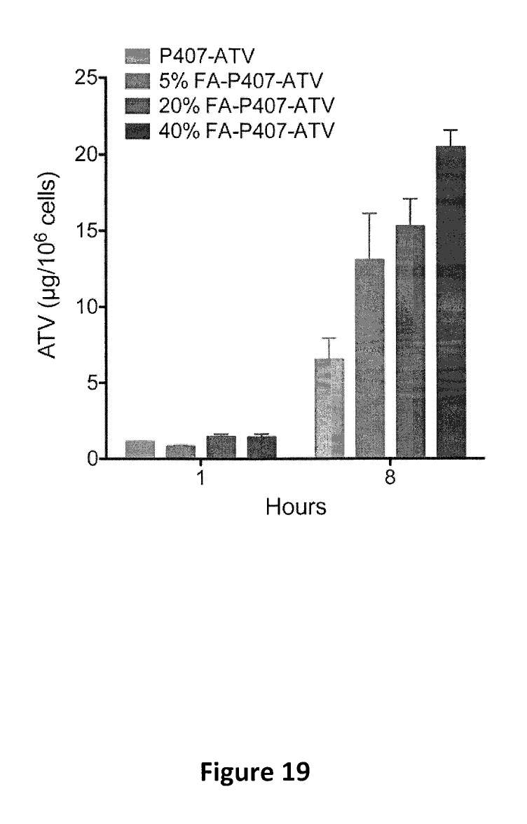

Because of the high CMC profile of P188, FA-P188-ATV nanoformulations will contain significantly amount of FA-P188 unimers that do not perform the targeting task of ATV nanosuspensions. P407, which has a lower CMC, was then selected as an alternative excipient to formulate ATV. This polymer was also modified with folic acid to prepare FA-P407-ATV nanosuspensions, and the difference in MDM uptake of ATV nanosuspensions containing various percentages of FA-P407 was determined under the same condition of FA-P188-ATV. As shown in FIG. 19, the uptake of ATV nanoformulations containing 5, 20 or 40% FA-P407 (H3020) was greater than the uptake of the unmodified P407-ATV nanosuspensions, and was dependent on the percent of FA-P407. Uptake of the formulation containing 40% FA-P407 was 5-fold greater at 8 hours than uptake of the ATV nanoformulation containing only un-modified P407.

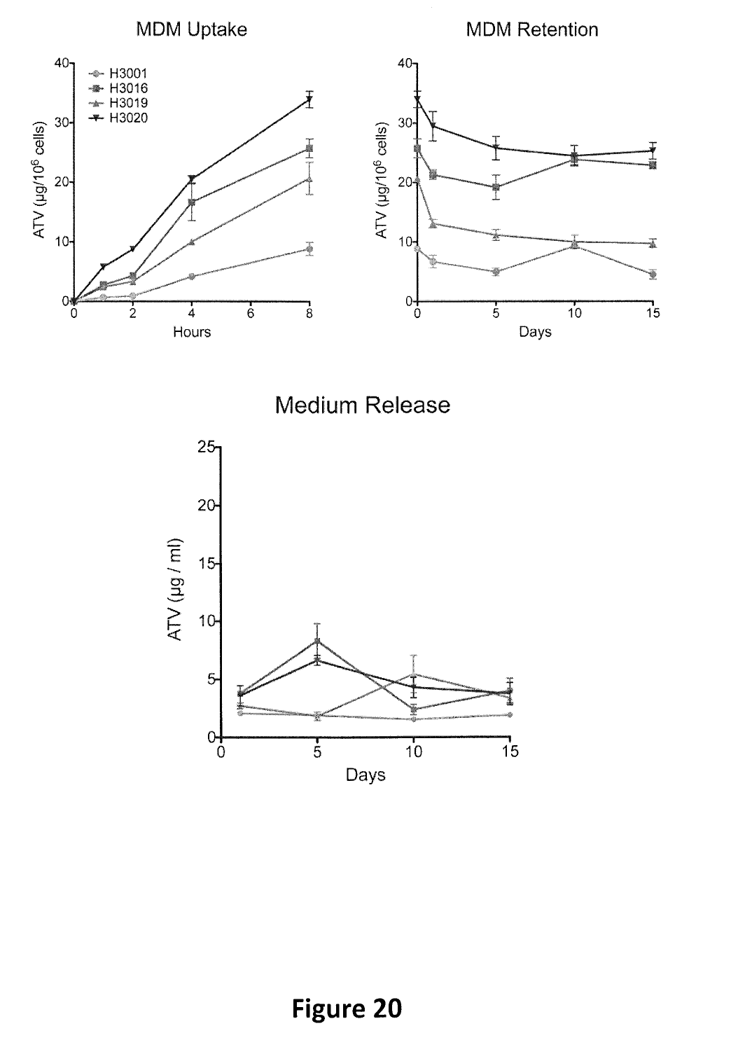

Based upon these results, ATV nanosuspensions containing P188 alone (H3001), 20% FA-P188 (H3016), P407 alone (H3019), or 40% FA-P407 (H3020) were selected for further studies to directly compare MDM uptake over 8 hours and their retention and release over 15 day (FIG. 20). Uptake of the P407-coated ATV nanosuspensions was enhanced 2.3-fold than uptake of the p188-coated particles after 8 hours (20.7 μg/10⁶ cells vs. 8.8 μg/10⁶ cells). Folate decoration of ATV nanosuspensions increased MDM uptake by 2.9- (P188) or 1.6-fold (P407) versus non-decorated ATV nanosuspensions. The retention profiles of ATV nanosuspensions through 15 days were similar for all formulations investigated, but the retention of FA-ATV nanosuspensions in MDM was significantly higher than that of non-FA decorated ATV nanosuspensions. During the first 24 hours cellular release, MDM lost 24%, 37%, 17% and 13% of drug following loading of H3001, H3019, H3016 and H3020, respectively, and cell ATV levels remained relatively constant and their release into the medium was sustained through 15 days post-treatment for all formulations (FIG. 20). At 15 days, cell drug levels were from 67% (H3001) to 100% (H3016) of drug levels at day 1. These results indicate that these formulations have sustained antiretroviral effect.

### Antiretroviral Efficacy of ATV Nanosuspensions

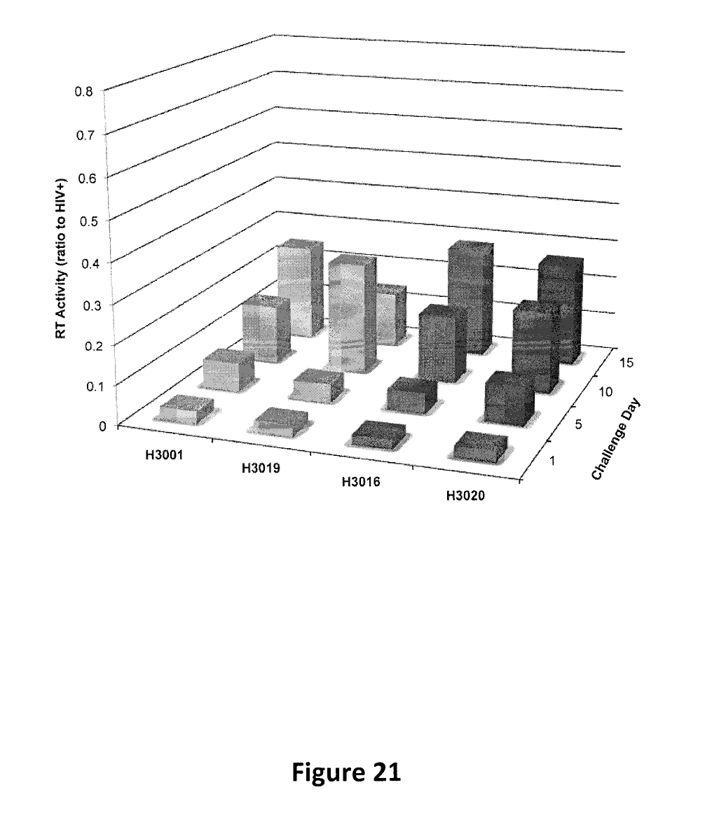

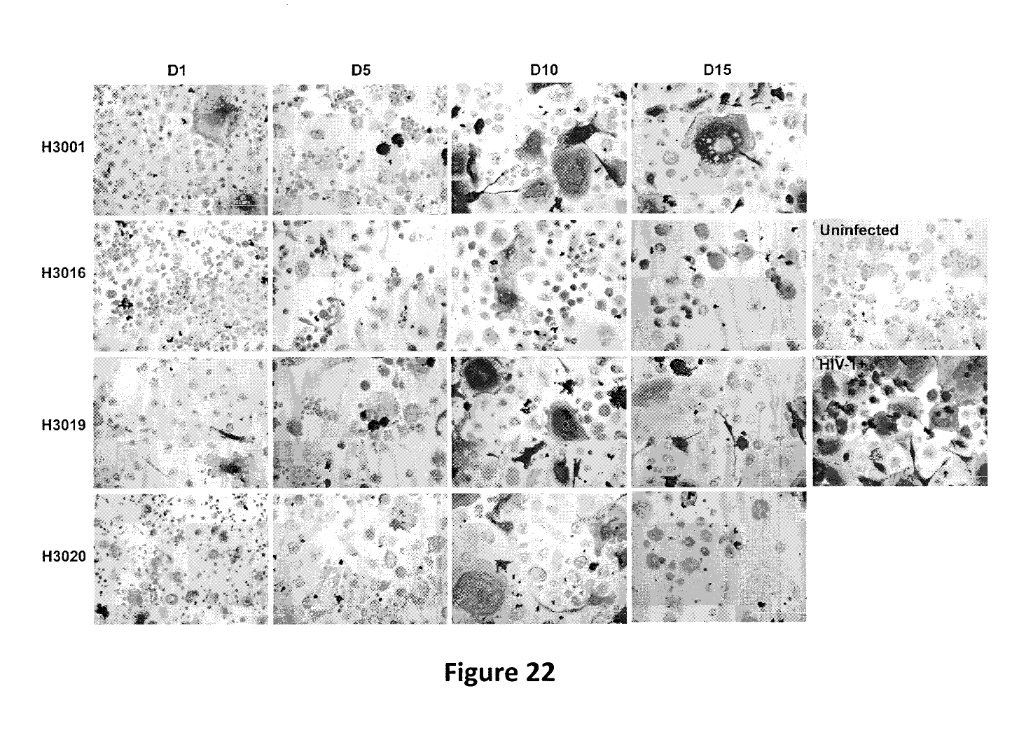

To determine antiretroviral efficacy of the formulations, ATV nanosuspensions containing P188 alone (H3001), 20% FA-P188 (H3016), P407 alone (H3019), or 40% FA-P407 (H3020) were selected for these studies. MDM were loaded with ATV nanosuspensions for 8 hours and then challenged with HIV-1_{ADA} virus 1, 5, 10, or 15 days after ATV nanosuspensions loading. Ten days after viral challenge the reverse transcriptase activity in the culture medium and HIV-1 p24+ staining in the cells was determined. HIV-1 viral infection was inhibited equally by all formulations. RT activity was inhibited by 70-90% when viral challenge occurred 10 days after ATV nanosuspensions treatment and by greater than 70% when viral challenge occurred 15 days after nanoparticle treatment (FIG. 21). Expression of p24 antigen verified the viral inhibition observed for RT activity (FIG. 22). Little p24+ staining (brown stain) was observed in cells challenged with virus 1 and 5 days after ATV nanosuspensions treatment. Viral challenge at 10 and 15 days after ATV nanosuspensions treatment resulted in some evident p24 staining in these cells. These results together indicate that ATV nanosuspensions decorated with folate-modified poloxamers have similar antiviral efficacy to particles coated with unmodified poloxamers.

### Example 4

### Materials and Methods

### Synthesis of Active Ester (pentynoic acid 2,5-dioxo-pyrrolidin-1-yl ester, 9)

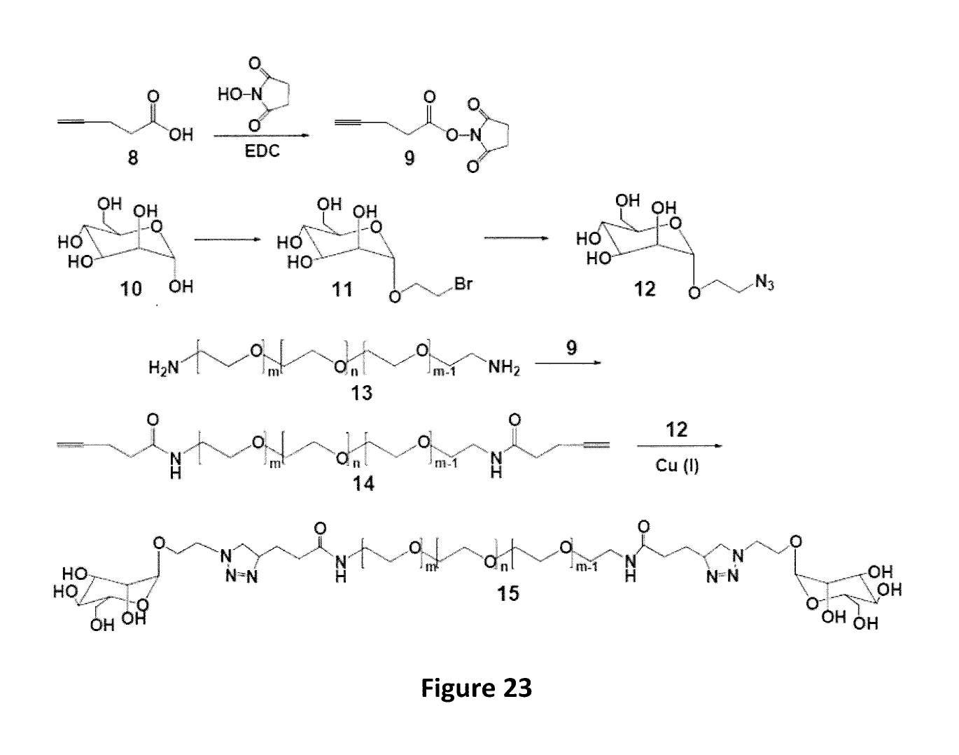

1.0 g (10 mmol) of 4-pentynoic acid was dissolved in 40 ml CH₂Cl₂. 1.27 g (11 mmol) of N-hydroxysuccinimide (NHS) was added (see FIG. 23). Then 1-ethyl-3-(3-dimethylaminopropyl)carbodiimide hydrochloride (EDC) was added (2.11 g, 11 mmol). The reaction was stirred at room temperature overnight. The reaction mixture was extracted with brine for 3 times. The organic layer was evaporated and dried to get the pure product. Yield: 1.5 g.

### Synthesis of 2-Bromoethyl-O-α-D-mannopyranoside (11)

1.5 g of Amberlite™ IR-120 was suspended in 11.5 ml of 2-bromoethanol (20.3 g; 0.164 mole). The mixture was heated at 90° C. in a flask equipped with condenser. After 30-40 minutes, about 1.5 g D-mannose (0.0083 mole) was added in a single portion. The reaction mixture was stirred at the same condition for 3 hours and then cooled to room temperature and filtrated. The solvent was removed under vacuum. The resulting sticky residue was dissolved in methanol and 5 g of silica gel was added to the flask to make a suspension. Solvent was removed by evaporation upon reduce pressure. The silica-supported reaction mixture was loaded onto a column previously filled with SiO₂ and pre-eluted with ethyl acetate, and then with ethyl acetate:methanol, 19:1 (v/v). Yield: 61%.

### Synthesis of 2-Azidoethyl-O-α-D-mannopyranoside (12)

0.46 g of sodium azide (0.007 mole) and 1 g of 2-Bromoethyl-O-α-D-mannopyranoside (0.0035 mole) were dissolved in 3 ml of DW. Then 20 ml of acetone was added to mixture to form slightly turbid solution. Reaction was heated up to reflux upon stirring for 24 hours. Purification of reaction mixture was performed by column chromatography with ethyl acetate:methanol, 5:1 as eluent.

### Synthesis of Acetylene Terminated F127 (Acetylene-F127, 14)

NH₂-F127 (2.56 g, 0.2 mmol) was dissolved in 10 ml DCM, then 0.39 g (2 mmol) pentynoic acid 2,5-dioxo-pyrrolidin-1-yl ester was added into this solution. The reaction solution was stirred at room temperature for 2 days. The reaction solution was concentrated and then precipitated in ether. The crude product was then purified with LH 20 column. Yield: 1.9 g.

### Synthesis of Mannose Terminated F127 (Mannose-F127, 15)

Acetylene terminated F127 (1.25 g, 0.1 mmol), 2-Azidoethyl-O-α-D-mannopyranoside (100 mg, 0.4 mmol), stabilizing agent (8.7 mg, 20 μmol) and CuSO₄.5H₂O (5 mg, 20 μmol) was dissolved in 4 ml Methanol/H₂O with stirring. Argon was bubbled to remove oxygen, then sodium ascorbic acid (40 mg, 0.2 mmol) in 0.5 mL H₂O was added into this solution drop by drop. The reaction mixture was allowed to stir at room temperature for 2 days. Solvents were removed under vacuum. The crude product was purified by LH-20 column. Yield: 0.8 g.

### Preparation of Nanosuspensions

Mannose-F127 was suspended in 10 mM HEPES buffer solution (pH 7.8). Free based ATZ (0.1% by weight) was then added to surfactant solutions. The suspensions were agitated to homogeneous dispersions by using an Ultra-turrax™ T-18 rotor-stator mixer. The mixtures were then transferred to a NETZSCH MicroSeries Wet Mill along with 50 mL of 0.8 mm grinding media (zirconium ceramic beads). The sample was processed for about 1 hour at speeds of about 4 krpm to prepare NanoART with desired particle size.

### Results

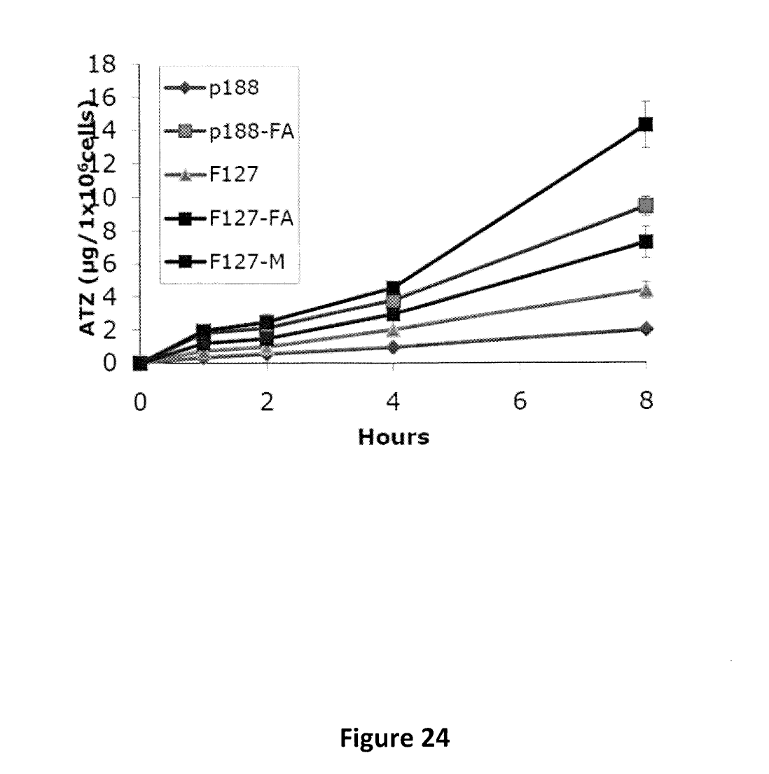

The nanoART uptake was assessed without medium change at different time points. Adherent MDM were washed with phosphate buffered saline (PBS) and collected by scraping into PBS. Cells were pelleted by centrifugation at 950×g for 10 minutes at 4° C. Cell pellets were briefly sonicated in 200 μl of methanol and centrifuged at 20,000×g for 10 minutes at 4° C. The methanol extract was stored at −80° C. until HPLC analysis. FIG. 24 shows that mannose ATV nanoAT are taken up by macrophage to greater levels than unlabeled ATV nanoART.

### Example 5

P188-ATV nanoART was administered to NSG mice at Day 0 and Day 7. Serum drug levels were analyzed at Days 1, 6 and 14 and tissue drug levels were analyzed at Day 14. No toxicity was evident from serum chemistry and histopathology evaluations. Serum levels at 7 days after the 2^{nd} 250 mg/kg dose (400 ng/ml) exceeded the minimum human therapeutic serum level of 150 ng/ml. ATV levels were highest in liver at 7 days after the 2^{nd} dose (1650 ng/g, w/250 mg/kg). Spleen, kidney, and lung ATV levels were equivalent (140-150 ng/g, w/250 mg/kg). Brain ATV levels were at the limit of quantitation. Serum and tissue levels were found to be dose-dependent.

Next, it was investigated whether a single nano ATV/RTV treatment administered to PBL-reconstituted NSG mice before HIV-1 infection provides sustained serum and tissue drug delivery and improved antiviral efficacy over an equivalent dose of free drug. Drug levels in serum 9 days after nanoART treatment exceeded by 2 logs the levels after free drugs. More specifically, nanoART (ATV, RTV, or EFV) retained significantly higher serum levels than free drug over a 14 day time course. Tissue drug levels were 100-1000-fold greater in nanoART treated mice than in free drug. CD4+ cell counts were not different in nanoART versus free drug-treated mice. NanoART treatment suppressed HIV-1 p24+ in spleen, which was not observed with free drug alone.

It was then determined whether nanoATV/RTV or nanoATV/RTV/EFV when administered in 2 weekly doses after HIV-1 infection to PBL-reconstituted NSG mice will provide therapeutic serum ATV levels, reservoir drug levels in lymphatic tissues, and antiretroviral efficacy. Briefly, PBL were administered to NSG mice at Day −7. The mice were challenged at Day −0.5 with HIV-1. NanoART was administered at Days 0 and 7 (nanoATV/RTV at 250 mg/kg or nanoATV/RTV/EFV at 100 mg/kg). Serum drug levels were examined at Days 1, 6, and 14 and tissue drug levels were analyzed at Day 14 along with CD4+ cells and p24 staining or RNA detection.

Therapeutic serum levels of ATV were achieved in mice treated with 2 doses of nanoART. Liver ATV levels were 2-fold higher than in normal NSG mice treated with a similar nanoATV/RTV dose. Spleen ATV levels were a log fold higher than liver ATV levels in the treated mice, unlike in normal NSG mice. Brain ATV levels were at the limit of quantitation. CD4+ cells and CD4+ CD8+ cell ratios were similar to uninfected mice following nanoART treatment of HIV-1 infected mice. However, nanoATV/RTV and nanoATV/RTV/EFV were both protective against HIV-1 infection in these mice (both therapies reduced p24 levels to almost undetectable levels).

Mice were also administered nanoparticles or free drug at only 10 mg/kg by SC injection. As seen in Table 4, this low dose of nanoparticles led to surprisingly high levels of drug concentration in vivo, superior to free drug.

TABLE 4
Plasma concentration of ATV, RTV, or EFV after 10 mg/ml
administration of nanoparticle or free drug (FD).

| | | | | |
|---|---|---|---|---|
| Time | NP-P188 | | FD | |
| (hr) | ng/ml | SEM | ng/ml | SEM |

ATV

| | | | | |
|---|---|---|---|---|
| 0.25 | 14.35 | 1.80 | 1148.59 | 402.58 |
| 1 | 16.11 | 1.00 | 804.50 | 319.99 |
| 3 | 23.71 | 3.52 | 2061.60 | 111.86 |
| 6 | 41.58 | 3.52 | 2099.06 | 111.86 |
| 10 | 321.55 | 105.08 | 638.86 | 346.81 |
| 24 | 94.87 | 13.45 | 9.85 | 6.34 |
| 48 | 40.88 | 9.25 | 4.38 | 1.40 |
| 96 | 62.27 | 17.56 | 4.30 | 1.01 |
| 168 | 12.72 | 1.52 | 1.74 | 0.50 |
| 336 | 5.03 | 0.50 | 0.00 | 0.00 |

RTV

| | | | | |
|---|---|---|---|---|
| 0.25 | 5.50 | 0.61 | 1079.63 | 507.61 |
| 1 | 12.87 | 1.60 | 492.03 | 199.43 |
| 3 | 57.53 | 8.74 | 710.50 | 59.60 |
| 6 | 106.73 | 8.74 | 551.30 | 59.60 |
| 10 | 170.10 | 22.97 | 69.08 | 17.18 |
| 24 | 356.03 | 71.31 | 5.91 | 1.71 |
| 48 | 437.00 | 58.29 | 2.36 | 0.80 |
| 96 | 752.33 | 177.24 | 4.94 | 1.57 |
| 168 | 2.59 | 0.97 | 2.21 | 0.43 |
| 336 | <LLOQ | 0.00 | 0.00 | 0.00 |

EFV

| | | | | |
|---|---|---|---|---|
| 0.25 | 2.73 | 1.06 | 301.47 | 150.68 |
| 1 | 5.48 | 0.83 | 123.74 | 60.03 |
| 3 | 15.99 | 2.30 | 155.36 | 12.69 |
| 6 | 95.47 | 2.30 | 212.77 | 12.69 |
| 10 | 298.00 | 25.28 | 39.99 | 9.09 |
| 24 | 366.33 | 42.19 | 7.10 | 2.09 |
| 48 | 358.00 | 52.74 | 2.17 | 0.66 |
| 96 | 389.67 | 103.35 | 2.99 | 1.02 |
| 168 | 2.06 | 0.25 | 2.57 | 0.14 |
| 336 | 2.59 | 0.19 | <LLOQ | 0.00 |

Lastly, it was determined whether nanoATV/RTV with folate-modified polymer as the excipient provides increased serum ATV drug levels, increased lymphatic tissue ATV levels and improved therapeutic efficacy. Folate-P407 ATV nanoART was administered to PBL-reconstituted NSG mice as described above after HIV-1 challenge. Spleen and lung ATV levels were similar to that in animals treated with P188-nanoATV/RTV. Kidney, liver, and brain ATV levels were ˜5-fold lower, ˜5-fold higher, and ˜10-fold higher, respectively, in mice treated with folate-modified nanoART than unmodified nanoART. CD4+ cell counts and CD4+/CD8+ cell ratios were increased to levels observed in uninfected mice. HIV-1 p24+ cells and RNA in spleen were decreased to nearly undetectable levels in folate-modified nanoART treated mice.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A nanoparticle comprising at least one therapeutic agent and at least one surfactant, wherein said nanoparticle is crystalline.

2. The nanoparticle of claim 1, wherein said surfactant coats a crystal of said therapeutic agent.

3. The nanoparticle of claim 1 which is rod shaped or rounded.

4. The nanoparticle of claim 1, wherein the z-average diameter is about 100 nm to 1 μm.

5. The nanoparticle of claim 1, wherein said surfactant comprises an amphiphilic block copolymer.

6. The nanoparticle of claim 5, wherein said amphiphilic block copolymer comprises at least one block of poly(oxyethylene) and at least one block of poly(oxypropylene).

7. The nanoparticle of claim 1, wherein said surfactant is selected from the group consisting of poloxamer 188, poloxamer 407, polyvinyl alcohol (PVA), 1,2-distearoyl-phosphatidyl ethanolamine-methyl-polyethyleneglycol conjugate-2000 (mPEG₂₀₀₀DSPE), sodium dodecyl sulfate (SDS), and 1,2-dioleoyloxy-3-trimethylammoniumpropane (DOTAP).

8. The nanoparticle of claim 1, wherein said surfactant is linked to at least one targeting ligand.

9. The nanoparticle of claim 8, wherein said targeting ligand is a macrophage targeting ligand.

10. The nanoparticle of claim 9, wherein said macrophage targeting ligand is folate.

11. The nanoparticle of claim 1, wherein said therapeutic agent is an antiretroviral.

12. The nanoparticle of claim 1, wherein said therapeutic agent is selected from the group consisting of atazanavir (ATV), efavirenz (EFV), indinavir (IDV), and ritonavir (RTV).

13. The nanoparticle of claim 12, wherein said therapeutic agent is selected from the group consisting of EFV, IDV, and RTV.

14. The nanoparticle of claim 1, wherein said surfactant is charged.

15. The nanoparticle of claim 14, wherein said surfactant is negatively charged.

16. The nanoparticle of claim 1, wherein said nanoparticle comprises at least about 95% therapeutic agent.

17. The nanoparticle of claim 16, wherein said nanoparticle comprises at least about 99% therapeutic agent.

18. A composition comprising at least one nanoparticle of claim 1 and at least one pharmaceutically acceptable carrier.

19. A method for treating or inhibiting an HIV infection in a subject in need thereof, said method comprising administering to said subject at least one composition of claim 18, wherein the therapeutic agent is an anti-HIV compound.

20. The method of claim 19, further comprising the administration of at least one additional anti-HIV compound.

21. The method of claim 19, wherein said targeting ligand is a macrophage targeting ligand.

22. The method of claim 21, wherein said macrophage targeting ligand is folate.

23. The method of claim 19, wherein said nanoparticle is administered at about 10 mg/kg or less.
